SEQUENCE LISTING (54 sequences)

SEQ ID NO: 1 - protein, 4 aa, Artificial Sequence
  Synthetic Construct
GFLG

SEQ ID NO: 2 - protein, 5 aa, Artificial Sequence
  Synthetic Construct
GFLFG

SEQ ID NO: 3 - protein, 4 aa, Artificial Sequence
  Synthetic Construct
GFFG

SEQ ID NO: 4 - protein, 5 aa, Artificial Sequence
  Synthetic Construct
YGGFL

SEQ ID NO: 5 - protein, 4 aa, Artificial Sequence
  Synthetic Construct
GGGF

SEQ ID NO: 6 - protein, 10 aa, Artificial Sequence
  Synthetic Construct
GFQGVQFAGF

SEQ ID NO: 7 - protein, 10 aa, Artificial Sequence
  Synthetic Construct
GFGSVQFAGF

SEQ ID NO: 8 - protein, 10 aa, Artificial Sequence
  Synthetic Construct
GFGSTFFAGF

SEQ ID NO: 9 - protein, 10 aa, Artificial Sequence
  Synthetic Construct
GLVGGAGAGF

SEQ ID NO: 10 - protein, 10 aa, Artificial Sequence
  Synthetic Construct
GGFLGLGAGF

SEQ ID NO: 11 - protein, 10 aa, Artificial Sequence
  Synthetic Construct
GFQGVQFAGF

SEQ ID NO: 12 - protein, 10 aa, Artificial Sequence
  Synthetic Construct
GFGSVQFAGF

SEQ ID NO: 13 - protein, 10 aa, Artificial Sequence
  Synthetic Construct
GLVGGAGAGF

SEQ ID NO: 14 - protein, 10 aa, Artificial Sequence
  Synthetic Construct
GGFLGLGAGF

SEQ ID NO: 15 - protein, 10 aa, Artificial Sequence
  Synthetic Construct
GFGSTFFAGF

SEQ ID NO: 16 - protein, 4 aa, Artificial Sequence
  Synthetic Construct
KALA

SEQ ID NO: 17 - DNA, 25 bp, Artificial Sequence
  Synthetic Construct
uguggaugacugaguaccugadtdt

SEQ ID NO: 18 - DNA, 25 bp, Artificial Sequence
  Synthetic Construct
ucagguacucagucauccacadtdt

SEQ ID NO: 19 - DNA, 32 bp, Artificial Sequence
  Synthetic Construct
ttttttcttttttttctcccagcgtgcgccat

SEQ ID NO: 20 - protein, 16 aa, Diptera
RQIKIWFQNRRMKWKK

SEQ ID NO: 21 - protein, 16 aa, Artificial Sequence
  Table I Transduction Peptide
KKWKMRRNQFWVKVQR

SEQ ID NO: 22 - protein, 16 aa, Artificial Sequence
  Table I Transduction Peptide
RRWRRWWRRWWRRWRR

SEQ ID NO: 23 - protein, 18 aa, Diptera
RQIKIWFQNRRMKWKKEN

SEQ ID NO: 24 - protein, 7 aa, Diptera
RRMKWKK

SEQ ID NO: 25 - protein, 13 aa, Human Immunodeficiency Virus
GRKKRRQRRRPPQ

SEQ ID NO: 26 - protein, 11 aa, Human Immunodeficiency Virus
YGRKKRRQRRR

SEQ ID NO: 27 - protein, 14 aa, Human Immunodeficiency Virus
PTSQSRGDPTGPKE

SEQ ID NO: 28 - protein, 17 aa, Human Immunodeficiency Virus
AVGAIGALFLGFLGAAG

SEQ ID NO: 29 - protein, 17 aa, Artificial Sequence
  Table I Transduction Peptide
GALFLGWLGAAGSTMGA

SEQ ID NO: 30 - protein, 30 aa, Artificial Sequence
  Table I Transduction Peptide
GALFLGFLGAAGSTMGAWSQPKSKRKVMPG

SEQ ID NO: 31 - protein, 24 aa, Artificial Sequence
  Table I Transduction Peptide
DRVIEVVQGAYRAIRNIPRRIRQG

SEQ ID NO: 32 - protein, 17 aa, Crocodile
MGLGLHLLVLAAALQGA

SEQ ID NO: 33 - protein, 27 aa, Crocodile
MGLGLHLLVLAAALQGAWSQPKKKRKV

SEQ ID NO: 34 - protein, 12 aa, Artificial Sequence
  Table I Transduction Peptide
PLSSIFSRIGDP

SEQ ID NO: 35 - protein, 27 aa, Artificial Sequence
  Table I Transduction Peptide
GWTLNSAGYLLGKINLKALAALAKKIL

SEQ ID NO: 36 - protein, 18 aa, Artificial Sequence
  Table I Transduction Peptide
RGGRLSYSRRRFSTSTGR

SEQ ID NO: 37 - protein, 16 aa, Mammalian
AAVALLPAVLLALLAP

SEQ ID NO: 38 - protein, 12 aa, Mammalian
AAVLLPVLLAAP

SEQ ID NO: 39 - protein, 15 aa, Human
VTVLALGALAGVGVG

SEQ ID NO: 40 - protein, 30 aa, Artificial Sequence
  Table I Transduction Peptide
VAYISRGGVSTYYSDTVKGRFTRQKYNKRA

SEQ ID NO: 41 - protein, 18 aa, Artificial Sequence
  Table I Transduction Peptide
KLALKLALKALKAALKLA

SEQ ID NO: 42 - protein, 30 aa, Artificial Sequence
  Table I Transduction Peptide
WEAKLAKALAKALAKHLAKALAKALKACEA

SEQ ID NO: 43 - protein, 24 aa, Mammalian
GLFEAIAGFIENGWEGMIDGGGYC

SEQ ID NO: 44 - protein, 7 aa, Artificial Sequence
  Table I Transduction Peptide
RRRRRRR

SEQ ID NO: 45 - protein, 26 aa, Artificial Sequence
  Table I Transduction Peptide
AAVALLPAVLLALLAPVQRKRQKLMP

SEQ ID NO: 46 - protein, 23 aa, Artificial Sequence
  Table I Transduction Peptide
MGLGLHLLVLAAALQGAKKKRKV

SEQ ID NO: 47 - protein, 14 aa, Artificial Sequence
  Table II Receptor Binding
TQPREEQYNSTFRV

SEQ ID NO: 48 - protein, 14 aa, Artificial Sequence
  Table II Receptor Binding
DGCSKAPKLPAALC

SEQ ID NO: 49 - protein, 8 aa, Artificial Sequence
  Table II Receptor Binding
YGGFLRRG

SEQ ID NO: 50 - protein, 3 aa, Artificial Sequence
  Table II Receptor Binding
YEE

SEQ ID NO: 51 - protein, 6 aa, Artificial Sequence
  Table II Receptor Binding
ZDFLFG

SEQ ID NO: 52 - DNA, 18 bp, Artificial Sequence
  ODN aldehyde
tctcccagcgtgcgccat

SEQ ID NO: 53 - RNA, 21 nt, Artificial Sequence
  Sense siRNA to bcl-2
uguggaugacugaguaccuga

SEQ ID NO: 54 - RNA, 21 nt, Artificial Sequence
  Antisense siRNA to bcl-2
ucagguacucagucauccaca

(19) United States
(12) Patent Application Publication
(10) Pub. No.: US 2006/0040879 A1
(43) Pub. Date: Feb. 23, 2006

(54) Chloroquine coupled nucleic acids and methods for their synthesis

(76) Inventor: Kenneth M. Kosak, West Valley City, UT (US)

Correspondence Address: KENNETH M. KOSAK, 3194 S. 4400 W., West Valley City, UT, 84120, US

(21) Appl. No.: 10/923,112

(22) Filed: Aug. 21, 2004

(51) Int. Cl. A61K 48/00; C07H 21/04

(52) U.S. Cl. 514/44; 536/23.7

(57) ABSTRACT

This invention discloses compositions and methods for preparing chloroquine-coupled nucleic acid compositions. The prior art has shown that chloroquines given as free drug in high enough concentration, enhances the release of various agents from cellular endosomes into the cytoplasm. The purpose of these compositions is to provide a controlled amount of chloroquine at the same site where the nucleic acid needs to be released, thereby reducing the overall dosage needed. The compositions comprise a chloroquine substance coupled to a nucleic acid directly or through a variety of pharmaceutical carrier substances. The carrier substances include polysaccharides, synthetic polymers, proteins, micelles and other substances for carrying and releasing the chloroquine compositions in the body for therapeutic effect. The compositions can also include a biocleavable linkage for carrying and releasing nucleic acids for therapeutic or other medical uses. The invention also discloses nucleic acid carrier compositions that are coupled to targeting molecules for targeting the delivery of nucleic acids to their site of action.

### RELATED PATENT APPLICATION

This is a continuation-in-part application of a U.S. patent application entitled:

“NUCLEIC ACID CARRIER COMPOSITIONS AND METHODS FOR THEIR SYNTHESIS”, inventor Ken. M. Kosak, filed Jun. 28, 2004. The contents of that application are incorporated herein.

### TECHNICAL FIELD OF THE INVENTION

This invention discloses chloroquine compositions for pharmaceutical and research use that include covalent and noncovalent linkages between nucleic acids and chloroquine or chloroquine analogs or derivatives (chloroquines or chloroquine substances). The composition can also include various carrier substances to which both the chloroquine and nucleic acid are coupled to produce a carrier composition (carrier). The carrier substances include polysaccharides, synthetic polymers, proteins, micelles and other substances for carrying and releasing the chloroquine compositions in the body for therapeutic effect.

Preferred carrier compositions contain biocleavable linkages that release the nucleic acids and chloroquines under controlled conditions. The carrier compositions can also include targeting molecules for targeting the delivery of nucleic acids and chloroquines to their site of action. The invention also discloses methods for preparing the carrier compositions.

### DESCRIPTION OF THE PRIOR ART

Active agents used in various therapies such as treatment for cancer, heart disease and infectious disease, hold great promise for curing or reducing the symptoms of many diseases.

An important category of active agents includes nucleic acids. Nucleic acid therapies such as gene therapy and especially antisense nucleic acid therapy also hold great promise for the treatment of many diseases and gene-related disorders.

However, when active agents including nucleic acids are administered in their “free” form, they frequently suffer from degradation after uptake by target cells. This degradation is frequently due to the nucleic acids collecting in cellular endosomes and/or lysosomes where chemical and enzymatic degradation is very efficient.

In the prior art, active agents have been conjugated to various particulate carriers and have been encapsulated into liposomes, micelles and nanoparticles where they are protected from serum degradation. The prior art also employs a variety of chemistries for covalent coupling of nucleic acids and other active agents to molecular carriers that include polymers such as dextrans or PEG. Such carriers may also include targeting moieties such as antibodies, polypeptides and other substances to direct the nucleic acids to selected target cells.

However, when nucleic acids enter the cell, they frequently end up sequestered in lysosomes where they are unable to escape. For nucleic acids, the prior art has tried to solve this problem through the use of cationic substance such as polyethylenimine (PEI). PEI is able to neutralize the lysosome and facilitate the release of the nucleic acid. However, PEI is known to be toxic and so far has not been FDA approved for use in humans.

It is well known in the prior art that “lysosomotropic” agents such as chloroquines are useful in releasing substances from lysosomes in tissue culture and thereby improving transfection with DNA. However, there is no disclosure of coupling chloroquines to DNA.

In the prior art it is known that some infectious disease organisms can survive in the acidic environment of cellular lysosomes where certain macrolide antibiotics have low activity. S. T. Donta in Medical Sci. Monitor 9, 136-142 (2003) reported that by treating patients with hydroxychloroquine in combination with certain macrolide drugs, the treatment of lyme disease was improved over use of these drugs alone. However, there is no disclosure or suggestion of coupling chloroquines to the macrolides.

There are several U.S. patents disclosing chloroquine for use against a variety of diseases either alone or in combination with other drugs. For instance, U.S. Pat. No. 4,181,725 and A. M. Krieg, et al, U.S. patent application 20040009949 discloses the use of chloroquine for treating various autoimmune diseases in combination with inhibitory nucleic acids. Also of interest are U.S. Pat. Nos. 5,736,557 and 6,417,177 where several chloroquine derivatives are disclosed. However, nothing in the prior art discloses or suggests the chloroquine-coupled compositions claimed in the present invention. Apparently, there are no nucleic acid-coupled chloroquine compositions disclosed or suggested in the prior art.

This may be due to reports in the art of nucleic acids that teach away from its in vivo use due to chloroquine toxicity. For instance, J. M. Benns, et al, recently reported, “Although chloroquine has proven to aid in the release of the plasmid DNA into the cytoplasm, it has been found to be toxic and thus cannot be used in vivo.” (1^{st} paragraph, Bioconj. Chem. 11, 637-645, (2000). This problem is partly due to the fact that relatively high concentrations of free chloroquine are needed to reach the same site as the nucleic acid (i.e. plasmid DNA) in the endosome.

Surprisingly, it was found that at least one embodiment of the present invention solves this problem by coupling one or more chloroquine moieties directly to the DNA so that the chloroquine has to be at the same site. Therefore, every moiety of nucleic acid, such as DNA, is automatically associated with the required amount of chloroquine to benefit from its action. There is no longer any need to use excess chloroquine because the compositions of the present invention automatically provide the benefits of chloroquine treatment at the same site as the nucleic acid. It will be apparent that the compositions of the instant invention provide other unexpected advantages such as cost savings and simple synthesis methods to allow administering more than one nucleic acid in a single dose.

### SUMMARY OF THE INVENTION

It will be understood in the art of nucleic acids that there are limitations as to which derivatives, coupling agents or other substances can be used with chloroquines to fulfill their intended function. The terms “suitable” and “appropriate” refer to substances or synthesis methods known to those skilled in the art that are needed to perform the described reaction or to fulfill the intended function. It will also be understood in the art of chloroquines and drug carriers that there are many substances defined herein that, under specific conditions, can fulfill more than one function. Therefore, if they are listed or defined in more than one category, it is understood that each definition or limitation depends upon the conditions of their intended use.

The prior art has shown that chloroquines given as free drug in high enough concentration, enhances the release of various agents from cellular endosomes into the cytoplasm. The purpose of these compositions is to provide a controlled amount of chloroquine at the same site where the nucleic acid needs to be released, thereby reducing the overall dosage needed.

The present invention is a chloroquine composition comprised of any suitable chloroquine substance coupled to a nucleic acid. The composition can also include various carrier substances to which both the chloroquine and nucleic acid are coupled to produce a carrier composition.

The carrier substances are divided into categories of suitable substances that include proteins, carbohydrates, polymers, grafted polymers and amphiphilic molecules as disclosed herein. The carrier composition can include a biodegradable linkage between the chloroquines and the carrier substance and/or between the nucleic acid and the carrier substance to provide controlled release of the chloroquines and/or nucleic acid after the carrier has reached its site of action. Optionally, one or several moieties can also be coupled to the carrier such as targeting molecules for targeting and transduction vectors disclosed herein to provide other desirable properties.

Any suitable synthesis method now used for preparing polymers conjugated to various moieties, with suitable modification, is applicable to the synthesis of this invention. A distinguishing property of this invention is that the chloroquines and nucleic acid are delivered together to their site of action.

For use as carriers, suitable polymers such as dextran or polyethylene glycol are commercially available in a variety of molecular masses. Based on their molecular size, they are arbitrarily classified into low molecular weight (Mw<20,000) and high molecular weight (Mw>20,000). In this invention, polymers of a molecular weight of 20,000 or greater are preferred when the purpose is to prevent rapid elimination of a polymer-coupled active agent due to renal clearance.

In one preferred embodiment, a suitable polyethylene glycol carrier has pendant reactive groups. The reactive groups are suitably conjugated to one or more chloroquines and one or more active agents such as nucleic acid, using various bifunctional cross-linking agents. The preferred embodiment may include biocleavable linkages as described herein. In another preferred embodiment, the carrier is suitably targeted by coupling suitable biorecognition molecules to the polymer carrier.

It has been discovered that the chloroquine compositions in the instant invention overcome many limitations of nucleic acid delivery in the prior art. The instant invention thereby provides new properties and unexpected advantages.

### DESCRIPTION OF THE PREFERRED EMBODIMENTS

For the purposes of disclosing this invention, certain words, phrases and terms used herein are defined below. Wherein certain definitions comprise a list of substances preceded by any grammatical form of the term “includes”, such substances are presented as examples taken from a group of substances known in the art to fit the said definition and the invention is not limited to the examples given.

### Chloroquine Substance

A chloroquine substance, is defined here as a usually (but not necessarily), lysosomotropic substance that includes, but is not limited to, quinoline compounds, especially 4-aminoquinoline and 2-phenylquinoline compounds and amino, thio, phenyl, alkyl, vinyl and halogen derivatives thereof. The most preferred chloroquine substances (sometimes called “chloroquines”), include chloroquine, hydroxychloroquines, amodiaquins (camoquines), amopyroquines, halofantrines, mefloquines, nivaquines, primaquines and quinone imines and chloroquine analogs or derivatives and wherein the (−)-enantiomers of chloroquine and hydroxychloroquine are most preferred.

Preferred chloroquine substances listed below with their chemical name, include, but are not limited to: 7-chloro-4-(4-diethylamino-1-methylbutylamino)quinoline (chloroquine); 7-chloro-4-(4-ethyl-(2-hydroxyethyl)-amino-1-methylbutylamino)quinoline (hydroxychloroquine); 7-fluoro-4-(4-diethylamino-1-methylbutylamino)quinoline; 4-(4-diethylamino-1-methylbutylamino)quinoline; 7-hydroxy-4-(4-diethylamino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethylamino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethylamino-1-butylamino)quinoline; 4-(1-carboxy-4-diethylamino-1-butylamino)quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethylamino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxyethyl)-amino-1-methylbutylamino-)quinoline7-hydroxy-4-(4-ethyl-(2-hydroxyethyl)-amino-1-methylbutylamino)quinoline; hydroxychloroquine phosphate; 7-chloro-4-(4-ethyl-(2-hydroxyethy-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino)quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 3-fluoro-4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof.

Preferred chloroquine substances include the agents, analogs and derivatives disclosed by D. J. Naisbitt, et al, in J. Pharmacol. Exp. Therapy 280, 884-893 (1997), and any quinolin-4-yl derivatives including N,N′-bis(quinolin-4-yl) derivatives disclosed in U.S. Pat. No. 5,736,557 and in references in the foregoing which are incorporated herein.

### Active Agents

Small Molecular Active Agents.

Small molecular active agents (or “small active agents” or “small drugs”), are defined here as limited to pharmaceutical chemicals and other substances with a molecular weight usually less than 1500 Daltons and are inhibitory, antimetabolic, therapeutic or preventive toward any disease (i.e. cancer, viral diseases, bacterial diseases, neurological diseases and heart diseases) or inhibitory or toxic toward any disease causing organism. Preferred small active agents are any therapeutic or prophylactic small drugs categorized in The Merck Index, Eleventh Ed., Merck & Co. Inc., Rahway N.J. (1989) and those listed by Cserhati, T., Anal. Biochem. 225(2), 328-332 (1995). Small active agents can be further limited to the following categories.

Chloroquine Combinative Active Agents.

Most preferred small active agents are “chloroquine combinative” active agents defined as active agents whose effectiveness or mode of action is amplified or improved or synergistic when used before, during or after treatment with chloroquine substances, defined herein. This includes, but is not limited to, any active agents used for prophylaxis or treatment against any infectious disease organisms, especially intracellular organisms that include viruses, bacteria, mycoplasma, protozoa and fungus. This also includes any active agents used for prophylaxis or treatment against any immunological or autoimmune diseases including rheumatoid arthritis, systemic lupus erythematosus (SLE), inflammatory bowel disease (IBD) graft-versus-host diseases and diabetes mellitus. Preferred active agents also include any active agents used for prophylaxis or treatment against any neurological diseases (i.e. multiple sclerosis, Alzheimer's, Parkinson's), heart diseases, prion diseases and cancers, especially drug resistant forms that rely on inhibition of apoptosis or on endosomal mechanisms to excrete active agents.

Preferred chloroquine combinative agents include, but are not limited to, any drugs or agents now used in combination with chloroquines, including cyclophosphamides and azathioprine for arthritis as disclosed by D. J. McCarty, et al, in J. Am. Med. Assoc. 248, 1718 (1982) and other combinative agents (including dapsone, 4,4′-sulfinyldianiline and penicillamine) and those disclosed in the Am. J. Med. 85, Suppl. 4A, 1-71 (1988) and any suitable derivatives of the foregoing agents. Also included are chloroquine combinatives including sulfadoxine, sulfisomidine and pyrimethamine, combinative with chloroquines such as mefloquine.

Preferred chloroquine combinative agents include certain neurological drugs such as deprenyls (selegilines), desmethyl deprenyls, pargylines, propargylines, rasagilines and CGP 3466 including derivatives disclosed by E. Kragten, et al, in The J. Biological Chem. 273, 5821-5828 (1998), and references therein which are herby incorporated herein.

Quinacrines.

Preferred small active agents also include certain quinacrines and quinacrine analogs and derivatives including those disclosed by C. Korth, et al, in PNAS 98, 9836-9841 (2001), among others. Also preferred are acridine agents containing hydroxyl groups such as Acramil® and 6-chloro-9-[4-[ethyl(2-hydroxyethyl)amino]-1-methylbutylamino]-2-methoxyacridine (hydroxyquinacrine), prepared by reacting a mixture of 6,9-dichloromethoxy acridine (0.5 mM) and N′-ethyl-N′-beta-hydroxyethyl-1, 4-pentadiamine (0.5 mM) in about 2 mL of phenol and heating to 120° C. for about 2 hours, based on the procedures of Korth, et al, (supra) and Surrey, et al, in JACS 72, 1814 (1950), with suitable modifications. Such agents containing hydroxyl groups can be suitably coupled through esterification to the desired moieties.

Toxins and Drugs of Abuse.

Small active agents also include any small toxins including aflatoxins, irinotecan, ganciclovir, furosemide, indomethacin, chlorpromazine, methotrexate, cevine derivatives and analogs including cevadines, desatrines, and veratridine; small drugs of abuse; alkaloids and narcotics among others.

Preferred small active agents that are also included are; intracellular transport agents such as bafilomycin, brefeldin, monensins and nordihydroguaiaretic acid among others;

Antibiotics and Antimicrobials.

Small active agents include but are not limited to therapeutic small drugs that include antifungal small drugs, antibacterial small drugs, antiviral small drugs, also included are various antibiotics including derivatives and analogs such as penicillin derivatives (i.e. ampicillin), anthracyclines (i.e. doxorubicin, daunorubicin, mitoxantrone), butoconazole, camptothecin, chalcomycin, chartreusin, chrysomicins (V and M), chloramphenicol, chlorotetracyclines, clomocyclines, ellipticines, filipins, fungichromins, griseofulvin, griseoviridin, guamecyclines, macrolides (i.e. amphotericins, chlorothricin, FK-506, L-865,818), methicillins, nystatins, chrymutasins, elsamicin, gilvocarin, ravidomycin, lankacidin-group antibiotics (i.e. lankamycin), mitomycin, teramycins, tetracyclines, wortmannins;

various anti-microbials including reserpine, spironolactone, sulfacetamide sodium, sulphonamide, thiamphenicols, thiolutins; also included are fungicides and pesticides;

various purine and pyrimidine derivatives and analogs including 5′-fluorouracil, 5′-fluoro-2′-deoxyuridine, and allopurinol;

Small Hormonal Agents.

Small active agents include but are not limited to, are prostaglandins; various steroidal compounds such as cortisones, estradiols, hydrocortisone, dehydroepiandrosterone (DHEA), testosterone, prednisolones, progesterones, dexamethasones, beclomethasones and other methasone derivatives, other steroid derivatives and analogs including digitoxins, digoxins, digoxigenins;

Other small active agents that are included, but are not limited to, are; vitamins A, B12, D3, K3, and folic acid, among others.

Anticancer Combinative Agents.

Preferred small active agents are “anticancer combinative agents” defined as any antineoplastic agents, prodrugs or cell growth inhibitors that are potentiated or enhanced when combined with other agents (i.e. antisense nucleic acids), that diminish or suppress drug resistance in cancers or cancer cells. These include, but are not limited to; cisplatins, taxanes including docetaxel (Taxotere®; Aventis Pharmaceuticals, Inc.) and paclitaxel (Taxol®; Bristol Myers Squibb), irinotecan (Camptosar®; Pfizer, Inc.), imatiinib (Gleevec®; Mesylate; Novartis), rituximab (Rituxan®; Genentech/IDEC), fludarabine (Fludara®; Berlex Laboratories, Inc), cyclophosphamide (Cytoxan®; Bristol Myers Squibb, Inc.), gemtuzumab ozogamicin (Mylotarg®; Wyeth-Ayerst, Inc.), cytosine arabinoside, dexamethasone and dacarbazine.

Protein and Peptide Active Agents.

The protein and peptide category of active agents are defined here as various pharmaceutical proteins, peptides, bioactive peptides and polypeptides that are inhibitory, antimetabolic, therapeutic or preventive toward any disease (i.e. cancer, syphilis, gonorrhea, influenza and heart disease) or inhibitory or toxic toward any disease causing agent. They include polypeptide hormones, insulins, interferons, interleukins, laminin fragments, tumor necrosis factors (TNF), cyclosporins, ricins, tyrocidines and bungarotoxins, among others.

Preferred protein and peptide active agents include pro-apoptotic peptides including the mitochondrial polypeptide called Smac/Diablo, or a region from the pro-apoptotic proteins called the BH3 domain and other pro-apoptotic peptides.

Pharmaceutical.

For the purposes of this invention, pharmaceutical or “pharmaceutical use” is defined as being limited to substances that are useful or potentially useful in therapeutic or prophylactic applications against diseases or disorders in humans, or any other vertebrate animals and in plants, especially plants of economic value. The most preferred substances defined as pharmaceutical are substances and/or compositions useful against viral, bacterial, fungal, protozoan, parasitic and other disease organisms, against cancers, autoimmune diseases, genetic diseases, heart diseases, neurological diseases and other diseases or disorders in humans and other vertebrates. Generally, but not necessarily, pharmaceutical substances are also biocompatible.

Biocompatible is defined here to mean substances that are suitably designed to be generally non-immunogenic, non-antigenic and will cause minimum undesired physiological reactions. They may or may not be degraded biologically and they are suitably “biologically neutral” for pharmaceutical applications due to suitably low non-specific binding properties.

Coupling.

For the instant invention, two distinct types of coupling are defined. One type of coupling can be through noncovalent, “attractive” binding as with a guest molecule and cyclodextrin, an intercalator and nucleic acid, an antigen and antibody or biotin and avidin. Such noncovalent coupling is binding between substances through ionic or hydrogen bonding or van der waals forces, and/or their hydrophobic or hydrophilic properties.

Unless stated otherwise, the preferred coupling used in the instant invention is through covalent, electron-pair bonds or linkages. Many methods and agents for covalendy coupling (or cross linking) of carrier substances including polyethylene glycol and other polymers are known and, with appropriate modification, can be used to couple the desired substances through their “functional groups” for use in this invention. Where stability is desired, the preferred covalent linkages are amide bonds, peptide bonds, ether bonds, and thio ether bonds, among others.

Functional Group.

A functional group or reactive group is defined here as a potentially reactive moiety or “coupling site” on a substance where one or more atoms are available for covalent coupling to some other substance. When needed, functional groups can be added to a carrier substance such as polyethylene glycol through derivatization or substitution reactions.

Examples of functional groups are aldehydes, allyls, amines, amides, azides, carboxyls, carbonyls, epoxys (oxiranes), ethynyls, hydroxyls, phenolic hydroxyls, indoles, ketones, certain metals, nitrenes, phosphates, propargyls, sulfhydryls, sulfonyls, vinyls, bromines, chlorines, iodines, and others. The prior art has shown that most, if not all of these functional groups can be incorporated into or added to the carrier substances of this invention.

Pendant Functional Group.

A pendant or “branched” functional or reactive group is defined here as a functional group or potentially reactive moiety described herein, that is located on a suitable polymer backbone such as pendant polyethylene glycol and “comb shaped” polymers, between the two ends. Preferably the pendant functional groups are located more centrally than peripherally.

Linkage.

A linkage is defined as a chemical moiety within the compositions disclosed that results from covalent coupling or bonding of the substances disclosed to each other. A linkage may be either biodegradable or non-biodegradable and may contain suitable “spacers” defined herein. Suitable linkages are more specifically defined below.

Coupling Agent.

A coupling agent (or cross-linking agent), is defined as a chemical substance that reacts with functional groups on substances to produce a covalent coupling, or linkage, or conjugation with said substances. Because of the stability of covalent coupling, this is the preferred method. Depending on the chemical makeup or functional group on a carrier substance, amphiphilic molecule, cyclodextrin, or targeting molecule, the appropriate coupling agent is used to provide the necessary active functional group or to react with the functional group. In certain preparations of the instant invention, coupling agents are needed that also provide a linkage with a “spacer” or “spacer arm” as described by O'Carra, P., et al, FEBS Lett. 43, 169 (1974) between a carrier substance and an intercalator or targeting molecule to overcome steric hindrance. Preferably, the spacer is a substance of 4 or more carbon atoms in length and can include aliphatic, aromatic and heterocyclic structures.

With appropriate modifications by one skilled in the art, the coupling methods referenced in U.S. Pat. No. 6,048,736 and PCT/US99/30820, including references contained therein, are applicable to the synthesis of the preparations and components of the instant invention and are hereby incorporated by reference.

Examples of energy activated coupling agents are ultraviolet (UV), visible and radioactive radiation that can promote coupling or cross linking of suitably derivatized substances. Examples are photochemical coupling agents disclosed in U.S. Pat. No. 4,737,454, among others. Also useful in synthesizing components of the instant invention are enzymes that produce covalent coupling such as nucleic acid polymerases and ligases, among others.

Useful derivatizing and/or coupling agents for preparing polymers are bifunctional, trifunctional or polyfunctional cross linking agents that will covalendy couple to the functional groups of suitable monomers and other substances.

Useful in this invention are coupling agents selected from the group of oxiranes or epoxides. Some preferred examples of oxiranes and epoxides include; epichlorohydrin, 1,4 butanediol diglycidyl ether (BDDE), bis(2,3-epoxycyclopentyl)ether 2,2′-oxybis(6-oxabicyclo[3.1.0]hexane) (BECPE), glycerol diglycidyl ether (GDE), trimethylolpropane triglycidyl ether (TMTE), tris(2,3-epoxypropyl)isocyanurate (TEPIC), glycerol propoxylate triglycidyl ether (GPTE), 1,3-butadiene diepoxide, triphenylolmethane triglycidyl ether, 4,4′-methylenebis (N,N-diglycidylaniline), tetraphenylolethane glycidyl ether, bisphenol A diglycidyl ether, bisphenol A propoxylate diglycidyl ether, bisphenol F diglycidyl ether, cyclohexanedimethanol diglycidyl ether, 2,2′-oxybis(6-oxabicyclo[3.1.0]hexane), polyoxyethylene bis(glycidyl ether), resorcinol diglycidyl ether, ethylene glycol diglycidyl ether (EGDE) and low molecular weight forms of poly(ethylene glycol) diglycidyl ethers or poly(propylene glycol) diglycidyl ethers, among others.

Other preferred derivatizing and/or coupling agents for hydroxyl groups are various disulfonyl compounds such as benzene-1,3-disulfonyl chloride and 4,4′-biphenyl disulfonyl chloride and also divinyl sulfone (J. Porath, et al, J. Chromatog. 103, 49-62, 1975), among others.

Most preferred coupling agents are also chemical substances that can provide the bio-compatible linkages for synthesizing the compositions of the instant invention. Covalent coupling or conjugation can be done through functional groups using coupling agents such as glutaraldehyde, formaldehyde, cyanogen bromide, azides, p-benzoquinone, maleic or succinic anhydrides, carbodiimides, ethyl chloroformate, dipyridyl disulfide and polyaldehydes.

Also most preferred are derivatizing and/or coupling agents that couple to thiol groups (“thiol-reactive”) such as agents with any maleimide, vinylsulfonyl, bromoacetal or iodoacetal groups, including any bifunctional or polyfunctional forms. Examples are m-maleimidobenzoyl-N-hydroxysuccinimide ester (MBS), succinimidyl 4-(N-maleimidomethyl)cyclohexane-1-carboxylate (SMCC), succinimidyl 4-(p-maleimidophenyl)butyrate (SMPB), dithiobis-N-ethylmaleimide (DTEM), 1,1′-(methylenedi-4,1-phenylene)bismaleimide (MPBM), o-phenylenebismaleimide, N-succinimidyl iodoacetate (SIA), N-succinimidyl-(4-vinylsulfonyl)benzoate (SVSB), and tris-(2-maleimidoethyl)amine (TMEA), among others.

Other coupling groups or agents useful in the instant invention are: p-nitrophenyl ester (ONp), bifunctional imidoesters such as dimethyl adipimidate (DMA), dimethyl pimelimidate (DMP), dimethyl suberimidate (DMS), methyl 4-mercaptobutyrimidate, dimethyl 3,3′-dithiobis-propionimidate (DTBP), and 2-iminothiolane (Traut's reagent);

bifunctional tetrafluorophenyl esters (TFP) and bifunctional NHS esters such as disuccinimidyl suberate (DSS), bis[2-(succinimido-oxycarbonyloxy)ethyl]sulfone (BSOCOES), disuccinimidyl (N,N′-diacetylhomocystein) (DSAH), disuccinimidyl tartrate (DST), dithiobis(succinimidyl propionate) (DSP), and ethylene glycol bis(succinimidyl succinate) (EGS), including various derivatives such as their sulfo-forms;

heterobifunctional reagents such as p-nitrophenyl 2-diazo-3,3,3-trifluoropropionate, N-succinimidyl-6(4′-azido-2′-nitrophenylamino)hexanoate (Lomant's reagent II), and N-succinimidyl-3-(2-pyridyldithio)propionate (SPDP), including various derivatives such as their sulfo-forms;

homobifunctional reagents such as 1,5-difluoro-2,4-dinitrobenzene, 4,4′-difluoro-3,3′-dinitrophenylsulfone, 4,4′-diisothiocyano-2,2′-disulfonic acid stilbene (DIDS), p-phenylene-diisothiocyanate (DITC), carbonylbis(L-methionine p-nitrophenyl ester), 4,4′-dithio-bisphenylazide and erythritolbiscarbonate, including derivatives such as their sulfo-forms;

photoactive coupling agents such as N-5-azido-2-nitrobenzoylsuccinimide (ANB-NOS), p-azidophenacyl bromide (APB), p-azidophenyl glyoxal (APG), N-(4-azidophenylthio) phthalimide (APTP), 4,4′-dithio-bis-phenylazide (DTBPA), ethyl 4-azidophenyl-1,4-dithiobutyrimidate (EADB), 4-fluoto-3-nitrophenyl azide (FNPA), N-hydroxysuccinimidyl-4-azidobenzoate (HSAB), N-hydroxysuccinimidyl-4-azidosalicylic acid (NHS-ASA), methyl-4-azidobenzoimidate (MABI), p-nitrophenyl-2-diazo-3,3,3-trifluoropropionate (PNP-DTP), 2-diazo-3,3,3-trifluoropropionyl chloride, N-succinimidyl-6(4′-azido-2′-nitrophenylamino)hexanoate (SANPAH), N-succinimidyl(4-azidophenyl)1,3′-dithiopropionate (SADP), sulfosuccinimidyl-2-(m-azido-o-nitobenzamido)-ethyl-1,3′-dithiopropionate (SAND), sulfosuccinimidyl (4-azidophenyldithio)propionate (Sulfo-SADP), sulfosuccinimidyl-6-(4′-azido-2′-nitrophenylamino)hexanoate (Sulfo-SANPAH), sulfosuccininidyl-2-(p-azidosalicylamido)ethyl-1,3′-dithiopropionate (SASD), and derivatives and analogs of these reagents, among others. The structures and references for use are given for many of these reagents in, “Pierce Handbook and General Catalog”, Pierce Chemical Co., Rockford, Ill., 61105.

Biocleavable Linkage or Bond.

For the instant invention, biocleavable linkages are defined as types of specific chemical moieties or groups that can be used within the compositions to covalently couple or cross-link a carrier substance with the nucleic acids, intercalators, active agents, targeting moieties, amphiphilic molecules and grafted polymers described herein. They may also be contained in certain embodiments of the instant invention that provide the function of controlled release of chloroquines and/or active agents. Biocleavable linkages or bonds are distinguishable by their structure and function and are defined here under distinct categories or types.

Ester Linkages. The ester bond is a preferred type that includes those between any acid and alcohol. Another preferred type is certain imidoesters formed from alkyl imidates. Also included are certain maleimide bonds as with sulfhydryls or amines used to incorporate a biocleavable linkage.

Acid Labile Linkages. Another category in this invention comprises biocleavable linkages that are more specifically cleaved after entering the cell (intracellular cleavage). The preferred biocleavable linkages for release of active agents and other moieties within the cell are cleavable in acidic conditions like those found in lysosomes. One type is an acid-sensitive (or acid-labile) hydrazone linkage as described by Greenfield, et al, Cancer Res. 50, 6600-6607 (1990), and references therein. Another type of preferred acid-labile linkage is any type of ortho ester, polyortho or diortho ester linkage, examples disclosed by J. Heller, et al., Methods in Enzymology 112, 422-436 (1985), J. Heller, J. Adv. Polymer Sci. 107, 41 (1993), M. Ahmad, et al., J. Amer. Chem. Soc. 101, 2669 (1979) and references therein. Also preferred are acid labile phosphonamide linkages disclosed by J. Rahil, et al, J. Am. Chem. Soc. 103, 1723 (1981) and J. H. Jeong, et al, Bioconj. Chem. 14, 473 (2003). Another preferred category is certain aldehyde bonds subject to hydrolysis that include various aldehyde-amino bonds (Schiff's base), or aldehyde-sulfhydryl bonds.

Cleavable Peptide Linkages. Another preferred category of biocleavable linkages is biocleavable peptides or polypeptides from 2 to 100 residues in length, preferably from 3 to 20 residues in length. These are defined as certain natural or synthetic polypeptides that contain certain amino acid sequences (i.e. are usually hydrophobic) that are cleaved by specific enzymes such as cathepsins, found primarily inside the cell (intracellular enzymes). Using the convention of starting with the amino or “N” terminus on the left and the carboxyl or “C” terminus on the right, some examples are: any peptides that contain the sequence Phe-Leu, Leu-Phe or Phe-Phe, such as Gly-Phe-Leu-Gly (GFLG), Gly-Phe-Leu-Phe-Gly and Gly-Phe-Phe-Gly, and others that have either of the Gly residues substituted for one or more other peptides. Also included are leucine enkephalin derivatives such as Tyr-Gly-Gly-Phe-Leu.

Biocleavable peptides also include cathepsin cleavable peptides such as those disclosed by J. J. Peterson, et al, in Bioconj. Chem., Vol. 10, 553-557, (1999), and references therein. Some examples are; GGGF, GFQGVQFAGF, GFGSVQFAGF, GFGSTFFAGF, GLVGGAGAGF, GGFLGLGAGF and most preferred are GFQGVQFAGF, GFGSVQFAGF, GLVGGAGAGF, GGFLGLGAGF, and GFGSTFFAGF. Also preferred are any peptides that contain parts of these cathepsin cleavable sequences.

Disulfide Linkage. A preferred category comprises the disulfide linkages that are well known for covalent coupling. For drug delivery they may be more useful for shorter periods in vivo since they are cleaved in the bloodstream relatively easily. A preferred type of biocleavable linkage is any disulfide linkages such as those produced by thiol-disulfide interchange (J. Carlsson, et al, Eur. J. Biochem. 59, 567-572, 1975).

Protected Disulfide Linkages. Another preferred type of biocleavable linkage is any “hindered” or “protected” disulfide bond that sterically inhibits attack from thiolate ions. Examples of such protected disulfide bonds are found in the coupling agents: S-4-succinimidyl-oxycarbonyl-α-methyl benzyl thiosulfate (SMBI) and 4-succinimidyloxycarbonyl-α-methyl-α-(2-pyridyldithio)toluene (SMPT). Another useful coupling agent resistant to reduction is SPDB disclosed by Worrell, et al., Anticancer Drug Design 1:179-188 (1986). Also included are certain aryldithio thioimidates, substituted with a methyl or phenyl group adjacent to the disulfide, which include ethyl S-acetyl 3-mercaptobutyrothioimidate (M-AMPT) and 3-(4-carboxyamido phenyldithio)proprionthioimidate (CDPT), disclosed by S. Arpicco, et al., Bioconj. Chem. 8 (3):327-337 (1997).

Azo Linkages. Another preferred type of biocleavable linkage in this invention are any suitable aromatic azo linkages that are cleavable by specific azo reductase activities in the colon as disclosed by J. Kopecek, et al., In: Oral Colon Specific Drug Delivery; D. R. Friend, Ed., pp 189-211 (1992), CRC Press, Boca Raton, Fla.

Controlled Release.

For this invention, controlled release (or “active release”) is defined as the release of chloroquine substances and/or an active agent from each other or from a carrier composition. Release of the active agent is by cleavage of certain biocleavable covalent linkages described herein that are used to couple the chloroquines or active agent to each other, or to the carrier substance, or to synthesize the carrier.

### Carrier Substance

The present invention is a composition comprised of a chloroquine substance coupled to a nucleic acid directly, or through said carrier substance. Preferably the carrier substance provides a biocompatible framework or “backbone” to which are coupled various moieties.

For the purposes of this invention, a carrier substance is defined as a molecular moiety suitable for pharmaceutical use (i.e. usually non antigenic), that is one of the starting materials used to synthesize the new carrier compositions of this invention.

This does not include antioxidants, adjuvants or so called pharmaceutical “carriers” or “drug vehicles” defined as pharmaceutical mixtures of solvents, dispersing agents, surfactants, excipients, or their combinations, that comprise a usually aqueous formulation for containing a drug or agent. However, a carrier composition of this invention may include a chemically modified form of a specific substance that has been used in such pharmaceutical mixtures. Also, a carrier composition of this invention may be a useful additive to certain pharmaceutical mixtures.

The carrier substances of this invention are limited by category to a variety of suitable substances including proteins, carbohydrates, grafted polymers and surfactants disclosed herein. The carrier substance can also include combinations of these suitable substances.

Plasma and Cellular Proteins.

Preferred plasma protein carrier substances include serum or plasma proteins including albumins, fibrinogens, globulins (gamma globulins, thyroglobulins), haptoglobins, histones, protamines and intrinsic factor including their derivatives such as their pegylated forms.

Preferred cellular protein carrier substances include cellular receptors, peptide hormones, enzymes, (especially cell surface enzymes such as neuraminidases) and their derivatives such as their pegylated forms. Preferred protein carrier substances include avidins, streptavidins, staphylococcal protein A, protein G and their derivatives including pegylated forms.

Antibodies. Preferred carrier substances include antibodies, including all classes of antibodies, monoclonal antibodies, chimeric antibodies, oxidized antibodies, pegylated antibodies, Fab fractions, fragments and derivatives thereof.

Oxidized Glycoproteins. A preferred category of carrier substances includes glycoproteins that have been suitably oxidized to provide aldehyde functional groups. These include oxidized forms of certain gamma globulins, alpha globulins, mucins, glycopeptides, ovomucoids and other mucoproteins.

Oxidized Antibodies. Another preferred carrier substance includes any oxidized forms of antibodies, including all classes of antibodies, monoclonal antibodies, chimeric antibodies, pegylated antibodies, fragments and derivatives thereof.

Peptide Carrier Substances.

Preferred carrier substances include any suitable peptides including the transduction vectors and receptor binding peptides defined herein. For example, the intercalators of this invention can be coupled to the amphipathic peptide KALA as disclosed by T. B. Wyman, et al, in Biochem. 36, 3008-3017 (1997), which may include derivatives and additional moieties as disclosed herein.

Carbohydrate Carrier Substances.

Preferred carrier substances are carbohydrates including polysaccharides that include alginates, amyloses, dextrans, dextran sulfates, chitosans, chitosan derivatives, chondroitins, chondroitin derivatives, cyclodextrins, cyclodextrin dimers, trimers and polymers including linear cyclodextrin polymers, gums (i.e. guar or gellan), hyaluronic acids, lectins, hemagglutinins, pectins, inulins and inulin derivatives, trisaccharides including raffinose and pegylated carbohydrates.

Grafted Polymers.

A grafted polymer is a category of carrier substances defined as a polymeric substance suitable for pharmaceutical use including copolymers and block polymers such as diblock or triblock copolymers prepared from a variety of monomers that are suitably coupled to produce a carrier substance as defined in the present invention.

Grafted polymers and copolymers can introduce other desirable properties such as a positive or negative net charge and hydrophobic properties. Preferred grafted polymers include cationic polymers and the amphiphilic molecules and polymers disclosed herein. Preferred grafted polymers are biocompatible, generally hydrophilic and have a molecular weight range from 1000 to 500,000 Daltons, preferably from 2,000 to 200,000 Daltons.

With suitable modification of the synthesis methods referenced by G. S. Kwon, IN: Critical Reviews in Therapeutic Drug Carrier Systems, 15(5):481-512 (1998) and by A. El-Aneed in J. Controlled Rel. 94, 1-14 (2004), including references therein, which are included herein, suitable grafted polymers can be synthesized for preparing the compositions of this invention. Included are diblock and triblock copolymer synthesis methods include ring-opening polymerization such as with PEO and various N-carboxyanhydride (NCA) monomers; polymerizations using triphosgenes and organo-metal (i.e. nickel) initiators (i.e. stannous octoate). Also useful are anionic, zwitterionic and free radical polymerizations and transesterifications, among others.

Some examples of suitable substances for use in grafted polymers are certain proteins, polypeptides, polyamino acids, glycoproteins, lipoproteins (i.e. low density lipoprotein), amino sugars, glucosamines, polysaccharides, lipopolysaccharides, amino polysaccharides, polyglutamic acids, poly lactic acids (PLA), polyacrylamides, poly(allylamines), lipids, glycolipids and suitable synthetic polymers, especially biopolymers as well as suitable derivatives of these substances. Also included as suitable substances are the polymers disclosed in U.S. Pat. No. 4,645,646.

Preferred grafted polymers include any polyethylene glycols (PEG), PEG derivatives, methoxy polyethylene glycols (mPEG), poly(ethylene-co-vinyl acetate) (EVAc), N-(2-hydroxypropyl)methacrylamides (HPMA), HPMA derivatives, poly(2-(dimethyl amino)ethyl methacrylate (DMAEMA), poly(D, L-lactide-co-glycolide) (PLGA), poly(polypropyl acrylic acid) (PPAA), poly(D,L-lactic-coglycolic acid) (PLGA), PLGA derivatives and poly(D,L-lactide)-block-methoxypolyethylene glycol (diblock) and any combinations, ratios or derivatives of these.

Also preferred grafted polymers are any copolymers that contain poly(ethylene oxide) (PEO) such as PEO-block-poly(L lysine), PEO-block-poly(aspartate), PEO-block-poly(beta-benzyl aspartate), PEO-block-poly(lactic acid), PEO-block-poly(L-lactic-coglycolic acid), poly(propylene oxide) (PPO), PEO-block-PPO and any combinations, ratios or derivatives of these.

Also preferred grafted polymers are any CD dimers, CD trimers, CD polymers and CD blocks, defined herein, poly cyanoacrylates such as poly(butyl cyanoacrylate), poly(isobutyl or isohexyl cyanoacrylate) and any combinations, ratios or derivatives of these.

Preferably grafted polymers also include any suitable combination of the polymers defined herein. Also preferred grafted polymers are comb shaped polymers including N-Ac-poly(L-histidine)-graft-poly(L-lysine) disclosed by J. M. Benns, et al, in Bioconj. Chem. 11, 637-645 (2000), and references

Amphiphilic Grafted Polymers. Amphiphilic grafted polymers are a preferred category of carrier substances that contain amphiphilic molecules. Amphiphilic molecules are defined as moieties suitable for pharmaceutical use that contain at least one hydrophilic (polar) moiety and at least one hydrophobic (nonpolar) moiety (i.e. surfactant). In certain embodiments of this invention, amphiphilic molecules including amphiphilic block polymers or copolymers are prepared for use as the carrier substance or as grafted polymers on the carrier substance.

In one embodiment, the desired targeting molecule or other substance can be coupled to available sites on the hydrophilic moieties of the amphiphilic molecule. Then, when the amphiphilic molecule is incorporated or “anchored” into a micelle with chloroquines and a nucleic acid coupled to a carrier, the targeting molecule is thereby noncovalendy coupled to the composition of the instant invention.

Most preferred are amphiphilic diblock or triblock copolymers prepared from a variety of monomers to provide at least one hydrophilic and one hydrophobic moiety. Amphiphilic cyclodextrin dimers, trimers and polymers as well as amphiphilic block copolymers containing CD dimers, trimers and polymers are included.

Preferred amphiphilic grafted polymers include any micelle-forming polymers or copolymers including PEG, PEG derivatives, PLGA, PLGA derivatives and poly (D,L-lactide)-block-methoxypolyethylene glycol (diblock), PEO, PEO derivatives or copolymers, PPO and PPO derivatives. Also preferred are any micelle-forming triblock copolymers (Pluronics) that contain PEO and PPO, such as PEO-block-PPO-block-PEO in various ratios. Specific examples are the F, L or P series of Pluronics including F-68, F-108, F-127, L-61, L-121, P-85, and any derivatives.

Cationic Grafted Polymers. Cationic grafted polymers are a preferred category of carrier substances defined as moieties suitable for pharmaceutical use that contain a net positive charge. In certain embodiments of this invention, cationic grafted polymers including cationic block polymers or copolymers are prepared for use as the carrier substance or as grafted polymers on the carrier substance.

Preferred cationic grafted polymers are comprised of or contain polyethylenimine (PEI), polyamidoamines (PAMAM), polylysine (PLL), poly-L-histidines (PLH) and poly arginines, among others.

Surfactant Carrier Substances.

Preferred surfactant carrier substances include suitable fatty acid derivatives, cholesterol derivatives including cholesterol hemisuccinate morpholine salts (CHEMS), gangliosides, phospholipids, pegylated phospholipids, dioleoylphosphatidyl choline (DOPC), dioleoylphosphatidyl ethanolamine (DOPE), any cationic lipids including 1,2-dioleoyl-3-trmethyl ammonium propane (DOTAP), 1,2-dioleyloxypropyl-3-trmethyl ammonium chloride (DOTMA), 1,2-dimyristyloxypropyl-3-dimethyl-hydroxyethyl ammonium bromide (DMRIE) and other suitable surfactants.

Liposome Carrier Particles.

Preferred carrier particles include liposomes as defined herein, including proteoliposomes and pegylated liposomes that contain the amphiphilic molecules as well as the protein, carbohydrate and polymer carrier particles defined herein. Said liposomes have the desired chloroquine substances, intercalators, targeting molecules, grafted polymers and other moieties coupled to the liposome through suitable covalent coupling that includes biocleavable linkages defined herein.

Coupling to the liposome can also be done through coupling a moiety to a suitable anchor substance such as an amphiphilic molecule or derivative, and then insertion of the anchor substance into the membrane, during or after liposome synthesis.

Liposomes are prepared from suitable amphiphilic molecules and the proteins, carbohydrates and grafted polymers of this invention using well known methods. For instance, a suitable method is disclosed by J. J. Wheeler, et al, in Gene Therapy 6, 271-281 (1999). The method employs detergent dialysis wherein the chloroquine-lipid conjugate of the present invention can be incorporated into any suitable mixture of amphiphilic molecules and suitable detergent. The detergent is then removed by dialysis to produce lipid vesicles containing the coupled chloroquine. The foregoing reference and references therein are hereby incorporated into this invention.

Nanoparticle Carriers.

Preferred nanoparticle carriers include the micelles, nanoparticles and dendrimers defined herein, including their pegylated forms and those that contain the amphiphilic molecules defined herein, as well as the proteins, carbohydrates and grafted polymers defined herein. Also included are micelles containing PEG, or poly(ethylene oxide) (PEO), or poly(propylene oxide) (PPO) such as those disclosed by S-F. Chang, et al, in Human Gene Therapy 15, 481-493 (2004), and references therein.

Micelles are prepared from block copolymers using well known methods. For instance, a suitable method is disclosed by P. L. Soo, et al, in Langmuir 18, 9996-10004 (2002) for polycaprolactone-block-poly(ethylene oxide). In that method, a suitable mixture of chloroquine-coupled lipid and the desired block copolymer are prepared in a suitable solvent such as DMF. Micellation is achieved by slowly adding water (2.5%/minute), with constant stiing, until the desired water content is achieved (i.e. 80-99%). The product is purified by exhaustive dialysis against water. The forgoing reference and references therein are hereby incorporated into this invention.

### Nucleic Acid Intercalators

A nucleic acid intercalator is defined as a substance that is capable of binding to nucleic acid defined herein, through attractive forces of intercalation including through van der Waals forces and/or hydrophobic attraction. For the purposes of this invention, preferred intercalators are limited by category to aromatic compounds that bind to single stranded nucleic acid (“hemi-intercalator”) or to double stranded (duplex) nucleic acid or to triple stranded (triplex) nucleic acid.

Nucleic acid intercalators are preferred that have a functional group available that also allows covalent coupling of the intercalator to chloroquines or a carrier substance without adversely affecting the nucleic acid intercalating or nucleic acid binding function of the intercalator. When such a functional group is not present, it can be added through suitable derivatization of the. intercalator. There are many types and categories of intercalators as described herein. Therefore, one skilled in the art can appreciate that some categories of intercalators are more preferred for the intended purpose of this invention than others.

### Covalent Coupling Nucleic Acid Intercalators

A covalent coupling nucleic acid intercalator is defined as a substance suitable for pharmaceutical use that, in addition to intercalating with nucleic acid, is also capable of forming covalent bonds with the nucleic acid when activated through a photoreactive or chemical process.

Photoreactive Intercalators.

The most preferred category of covalent coupling intercalators are the photoreactive intercalators including those of the furocoumarin family of compounds as disclosed by G. D. Cimino in Ann. Rev. Biochem. 54, 1151-1193 (1985), incorporated herein by reference.

Some preferred examples of photoreactive intercalators include psoralens, psoralen amines, hydroxyl psoralens (4′-hydroxymethyl psoralens), trioxsalens (4,5′,8-trimethylpsoralens), trioxsalen amines (4′-aminomethyl-4-5′-8-trimethyl psoralens), hydroxyl trioxsalens (4′-hydroxymethyl trioxsalens), methoxsalens, 5-methoxypsoralens, 8-methoxypsoralens, 4′-hydroxymethyl-4,5′,8-trimethylpsoralens, 4′-methoxymethyl-4,5′,8-trimethylpsoralens, 4′-chloromethyl-4,5′,8-trimethylpsoralens and 4′-N-phthalimidomethyl-4,5′,8-trimethylpsoralens.

Also preferred are any suitable amino, vinyl, sulfhydryl or phosphoramidite derivatives of psoralen or trioxsalen including 6-(4′-hydroxymethyl-4,5′,8-trimethylpsoralen)hexyl-1-O-(beta-cyanoethyl-N,N′-diisopropyl)phosphoramidite, among others. A preferred amino derivative is “psoralen amine” available from Sigma-Aldrich, St. Louis, Mo., 2003 Catalog #P 6100.

Also preferred are any suitable derivatives of psoralen or trioxsalen including biotinylated forms as is disclosed by C. Levenson, et al, in Methods in Enzymology 184, 577-583 (1990).

Also preferred are any suitable psoralen or trioxsalen active esters (i.e. N-hydroxysuccinimide, or 4-nitrophenyl), including 4′-[(3-carboxypropionamido)methyl]-4,5′,8-trimethylpsoralen N-hydroxysuccinimide ester, as is disclosed by M. A. Reynolds in Bioconj. Chem. 3, 366-374 (1992).

Also preferred are suitable amino acid derivatives of psoralen or trioxsalen such as aspartic acid-beta-(4′-aminomethyl-4,5′,8-trimethylpsoralen) disclosed by Z. Wang, et al, in JACS 117, 5438 (1995).

Also preferred are any suitable psoralen or trioxsalen derivatized with anhydride, carboxylate, chloroformate, tosylate or isothiocyanate functional groups.

Also preferred are any suitable psoralen or trioxsalen derivatives herein disclosed that include alkyl, or alkyl amino extensions, or spacer groups.

Anthraquinones.

Another category of nucleic acid intercalators includes photoreactive anthraquinone derivatives as disclosed by T. Koch, et al, in Bioconj. Chem. 11, 474-483 (2000).

Nucleic Acid Alkylating Agents.

Another category of nucleic acid intercalators includes alkylating agents such as p-azidophenacyl, duocarmycin A (i.e. pyrinamycins) and duocarmycin C. Also the agent (+)-CC-1065 and its analogs possessing the 1,2,9,9a-tetrahydrocyclopropa[1,2-c]benz[1,2-e]indol-4-one (CBI) alkylation subunit including 1-(chloromethyl)-5-dihydro-3H-benz[e]indole (seco-CBI) disclosed by A. Y. Chang, et al, JACS 122, 4856-4864 (2000), and naphthopyranone epoxides disclosed by K. Nakatani, et al, in JACS 123, 5695-5702 (2001) and references therein.

Another category of nucleic acid intercalators includes certain intercalators known to produce covalent nucleic acid complexes such as aflatoxin B oxide and certain pluramycin antibiotics (i.e. kapuramycin A).

### Non-Covalent Coupling Nucleic Acid Intercalators

A non-covalent coupling nucleic acid intercalator is defined as a substance suitable for pharmaceutical use that generally does not form covalent bonds with the nucleic acid, but is coupled through the forces of intercalation. The most preferred non-covalent coupling nucleic acid intercalators are those that form and maintain the strongest noncovalent bonds, especially under physiological or pharmaceutical conditions.

Acridine and Acridine Derivatives.

One category of nucleic acid intercalators includes acridine and acridine derivatives such as acridine orange and derivatives thereof, acridine carboxamides, 9-aniloacridine, 3-(9-acridinyl amino)-5-hydroxyethyl aniline (AHMA) derivatives and their alkylcarbamates, acroycines including 1,2-dihydroxy-1,2-dihydro acronycine and 1,2-dihydroxy-1,2-dihydro benzo[b] acronycine diesters, pyrimidol[5,6,1-de]acridines, pyrimidol[4,5,6,-kl]acridines, bis(amine-functionalized) 9-acridone-4-carboxamides, bis(amine-functionalized)acridine-4-carboxamides and pyrazolo[3,4,5-kl]acridine-5-carboxamides.

Also included are bis-acridines disclosed by May, et al, in PNAS, vol. 100, 3416-3421 (2003), and references therein, including bis-(6-chloro-2-methoxy-acridin-9-yl) and bis-(7-chloro-2-methoxy-benzo[b][1,5]-naphthyridin-10-yl) analogs such as (6-chloro-2-methoxy-acridin-9-yl)-(3-{4-[3-(6-chloro-2-methoxyacridin-9-ylamino)-propyl]-piperazin-1-yl}-propyl)-amine, N,N′-bis-(6-chloro-2-methoxy-acridin-9-yl)-1,8-diamino-3,6-dioxaoctane, and (1-{[4-(6-chloro-2-methoxy-acridin-9-ylamino)-butyl]-[3-(6-chloro-2-methoxy-acridin-9-ylamino)-propyl]-carbamoyl}-ethyl)-carbamic acid tert-butyl ester. Also included are quinacrines and covalent dimers of quinacrtine.

Anthracyclines and Derivatives.

Another category of nucleic acid intercalators includes anthracyclines such as nogalamycin, daunomycin and adriamycin (doxorubicin), mitoxantrone and ametantrone. Also included are ene-diyne antibiotics such as dynemycin.

Anthracenes and Derivatives.

Another category of nucleic acid intercalators includes anthracenes, phenylanthracenes and their derivatives, including anthraquinolyns,

Actinomycins and Derivatives.

Another category of nucleic acid intercalators includes actinomycins including actinomycins C, actinomycins D, 7-amino actinomycins and mitomycin C.

Aminoglycosides and Derivatives.

Another category of nucleic acid intercalators includes aminoglycosides such as neomycin B, kanamycin A, and tobramycin including derivatives such as their conjugates with 9-aminoacridine as are disclosed by Luedtke, et al, in Biochemistry Vol. 42, 11391-11403 (2003) and references therein. Also included are conjugates neo-N-acridine, neo-C-acridine, tobra-N-acridine, kana-N-acridine, neo-N-neo, tobra-N-tobra, neo-S-acridine, neo-neo, tobra-tobra, and kanaA-kanaA.

Porphyrins.

Another category of nucleic acid intercalators includes porphyrins, hematoporphyrins and derivatives, metal-free porphyrins such as H2TMpyP-4. Also included are four-coordinate metalloporphyrins such as CuTMpyP-4, NiTMpyP-4 and PdTMpyP-4 and [Ru(II)12S4dppz]Cl₂.

Pyrenes and Other Intercalators.

Another category of nucleic acid intercalators includes suitable pyrene intercalators including 1-O-(1-pyrenylmethyl)glycerol and derivatives thereof. Another category of nucleic acid intercalators includes ethidiums, propidiums, proflavins, ellipticines and 4,6′-diaminide-2-phenylindole (DAPI).

Another category of nucleic acid intercalators includes distamycin, berenil, Hoechst dyes including Hoechst 33258 and Hoechst 33342.

Covalent Intercalation Linkage.

A covalent intercalation linkage is defined for this invention as a composition wherein an intercalator is a fully covalent coupling agent between a nucleic acid and a carrier substance defined herein. Said intercalator is covalendy coupled to said carrier substance through suitable functional groups and/or through a covalent cross linking agent and also covalently coupled through “covalent intercalation” to said nucleic acid. Said covalent intercalation comprises intercalation with said nucleic acid and subsequent conversion of the intercalation binding to a covalent bond or coupling through chemical or photochemical means.

Non-Covalent Intercalation Linkage.

A noncovalent intercalation linkage is defined for this invention as a composition wherein an intercalator can be covalendy coupled as defined to said carrier substance but is noncovalently coupled only through the forces of intercalation to said nucleic acid.

### Nucleic Acids

For the purposes of this invention, “nucleic acids” are defined as a class of active agents that are limited by category to include any pharmaceutical nucleic acids, meaning useful or potentially useful in therapeutic or prophylactic applications in humans, or any other vertebrate animals and in plants. The most preferred nucleic acids defined as pharmaceutical are nucleic acid active agents against viral and other microbial diseases, against cancers, heart diseases, autoimmune diseases, genetic and other diseases or disorders in humans and other vertebrates. Also included are nucleic acid active agents against viral and other microbial diseases in plants. They also include specific DNA sequences used for gene therapy.

RNA

One category of nucleic acid active agents includes all types of single stranded or double stranded RNA (dsRNA), including antisense RNA, messenger RNA (mRNA) and transfer RNA (tRNA). Most preferred are any RNAs useful in RNA interference (RNAi) therapeutics such as small interfering RNAs (siRNA) and interfering dsRNA.

In one preferred embodiment for coupling dsRNA, the desired sense RNA single strand is first coupled by intercalation to a suitable carrier, then the antisense strand is hybridized with the sense strand on the carrier to form dsRNA. Alternatively, the desired sense RNA single strand is hybridized with the antisense strand to form dsRNA, which is then coupled by intercalation to a suitable carrier.

Also preferred are any micro RNAs (miRNA) and any antisense nucleic acids used to inactivate miRNA, such as antisense nucleic acids containing 2′-O-methyl groups, including those disclosed by Hutvagner, et al, PLOS Biol. 2, 10. 10371/Journal.pbio.0020114(2004) and Meister, et al, RNA 10, 544 (2004).

Also preferred nucleic acids are any ribozymes and hairpin ribozymes including those disclosed or referenced by Y. Lian, et al, in Gene Therapy, Vol. 6, 1114-1119 (1999).

Also preferred nucleic acids are any suitable plasmids and pCOR plasmids including those disclosed or referenced by F. Soubrier, et al, in Gene Therapy, Vol. 6, 1482-1488 (1999).

Also preferred are any riboswitches. Riboswitches are defined as metabolite-binding nucleic acids, or specific metabolite-binding nucleic acid sequences, such as in messenger RNAs that serve as sensors for modulation of gene expression or other functions. Some examples are described by M. Mandal, et al, Cell 113, 577 (2003), including references therein, all of which are incorporated by reference herein. Preferred riboswitches, or the specific metabolite-binding nucleic acid sequences, are those found in vertebrates, mammalian cells, bacteria and higher plants.

Also preferred are 5′ derivatized RNA, or 3′ derivatized RNA where the 5′ or 3′ ends have been capped, or labeled, or extended with additional nucleic acids, or amino acids, or a mutagen, or suitably derivatized in any way. Also preferred are “backbone derivatized” RNAs in which the sugar-phosphate “backbone” has been derivatized or replaced with “backbone analogues” which include phosphorothioate, phosphorodithioate, phosphoroamidate, alkyl phosphotriester, or methylphosphonate linkages or other backbone analogues. Such derivatized RNA includes any sense or antisense sequences.

The two strands of the siRNA duplex can be produced by standard protocols, and many of the chemical modifications that have been developed to improve classical antisense oligonucleotides can also be introduced into RNA (Braasch, D. A., et al, Biochem. 42, 7967, 2003). These modifications may improve the thermal stability, serum stability, cellular activity, or pharmacokinetic properties of RNA. Nucleic acids also include the proteins that make up the RNAi induced silencing complex (RISC).

Also preferred are any “modified ribose” nucleic acids which includes modification of the 2′ position of the ribose ring, including 2′-O-methyl (i.e. 2′-O-meRNA) (Monia, B. P., et al, (1993) J. Biol. Chem. 268, 14514), 2′-deoxy-2′-fluorouridine (Kawasaki, A. M., et al, (1993) J. Med. Chem. 36, 831) and any nucleic acids with the 2′-hydroxyl eliminated or modified.

Also preferred are “locked” nucleic acids (LNA) (Koshkin, A. A., et al, (1998) Tetrahedon 54, 3607 22-24), which contains a methylene linkage between the 2′ and the 4′ positions of the ribose. These modifications can increase stability to degradation by nucleases or improve thermal stability. Also included are nucleic acids that contain LNA, 2′-O-meRNA, or 2′-deoxy-2′-fluorouridine bases and also contain several consecutive DNA bases (i.e. if cleavage of RNA by RNAse H is desired).

DNA

One category of preferred nucleic acids includes all types of single stranded or double stranded DNA, and oligodeoxynucleotides. Preferred DNAs include any 5′ derivatized DNA, or 3′ derivatized DNA where the 5′ or 3′ ends have been capped, or labeled, or extended with additional nucleic acids, or amino acids, or a mutagen, or suitably derivatized in any way.

Preferred nucleic acids also include all types of inhibitory nucleic acids including those with a poly G motif and the sequences disclosed by A. M. Krieg, et al, U.S. patent application 20040009949, incorporated herein by reference.

Preferred nucleic acids also include enzymatic or RNA-cleaving DNA such as DNAzymes, including those disclosed by S. Schubert, et al in Nucleic Acids Res. 31, 5982 (2003), Ota, et al in Nucleic Acids Res. 26, 3385 (1998) and L. Zhang, et al, in Cancer Res. 62, 5463 (2002) and references in the foregoing, which are hereby incorporated herein.

Sense and Antisense Nucleic Acids.

Also preferred are any antisense nucleic acids that include phosphodiester antisense oligonucleotides (ON) and antisense oligodeoxynucleotides (ODN).

Also preferred are any sense and/or antisense “backbone derivatized” oligonucleotides or “backbone derivatized” oligodeoxynucleotides where the sugar-phosphate “backbone” has been derivatized or replaced with “backbone analogues” which include phosphorothioate (PS), phosphorodithioate, phosphoroamidate, alkyl phosphotriester, or methylphosphonate linkages or other “backbone analogues”. Such “backbone derivatized” sense and/or antisense oligonucleotides or oligodeoxynucleotides include those with non-phosphorous backbone analogues such as sulfamate, 3′-thioformacetal, methylene(methylimino) (MMI), 3′-N-carbamate, or morpholino carbamate.

Mixed Backbone Nucleic Acid Derivatives.

In one category of backbone derivatized nucleic acids (sense and/or antisense), only one section of the sugar-phosphate backbone has been derivatized or replaced with backbone analogues. One example of a preferred, but not limited to, ODN in this invention has the following sequence composition;

### Phosphodiester Extension|Phosphorothioate G3139 Antisense bcl2

5′-Amino-TT TTT TCT TTT TTT TCT CCC AGC GTG CGC CAT-3′

Another class of “mixed” backbone derivatized nucleic acids (sense and/or antisense) is where the sugar-phosphate backbone has been derivatized or replaced with backbone analogues in an alternating or mixed fashion. For instance, the base sequence of a mixed backbone ON or mixed backbone ODN would be comprised of short sections (i.e. one, two or more bases) of phosphodiester linkages alternating with sections of one or more backbone analog linkages such as phosphorothioate, or phosphorodithioate, or phosphoroamidate, or alkyl phosphotriester, or methylphosphonate linkages. These linkages can be in any desirable order or ratio in order to obtain the desired characteristics such as solubility, hydrophobicity, charge, etc. Preferably, such mixed backbone nucleic acids would allow an optimal balance in lower toxicity with higher efficacy and stability.

Capped Nucleic Acids.

One category also preferred is capped nucleic acids including phosphodiester antisense oligonucleotides, antisense ODNs and any sense or antisense backbone derivatized oligonucleotides or oligodeoxynucleotides where the 5′ or 3′ ends have been capped, or labeled, or extended with additional nucleic acids, or amino acids, or a mutagen.

Preferred examples of said capped antisense nucleic acids include 3′ capped oligonucleotides or oligodeoxynucleotides with hexylamine, 1,2-propanol, diethyleneglycol or 2,2-dimethyl-1,3-propanol coupled to their 3′ end, as disclosed by S. Dheur, et al, Antisense & Nucleic Acid Drug Dev. 9, 515-525 (1999), and references therein.

Hybrid Nucleic Acids.

Also preferred are any nucleic acid hybrids (i.e. RNA-DNA hybrids) including any sense or antisense “backbone derivatized” oligonucleotides or oligodeoxynucleotides where RNA and DNA are hybridized through complementary sequences to form double or triple strands. This includes any sense or antisense hybrids containing any type of 5′ derivatized RNA, or 3′ derivatized RNA, or 5′ derivatized DNA, or 3′ derivatized DNA where the 5′ or 3′ ends have been capped, or labeled, or extended with additional nucleic acids or amino acids, or suitably derivatized in any way.

Chimera Nucleic Acids.

One category also preferred is nucleic acid chimeras (i.e. RNA-DNA chimeras) wherein the sense or antisense nucleic acid strand is comprised of one or more sections of RNA and one or more sections of DNA grafted together. Said nucleic acid chimeras include those containing amino acids, or a mutagen, or any suitable polymer (i.e. PEG) or is suitably derivatized in any way.

Some preferred examples of synthetic oligonucleotides and ODNs are disclosed by J. F. Milligan, et al., J. Medicinal Chem. 36(14): 1923-1937 (1993) and Y. Shoji, et al., Antimicrob. Agents Chemotherapy, 40(7):1670-1675 (1996).

Also included are synthetic nucleic acid polymers including sense and/or antisense peptide nucleic acids (PNA) disclosed by Egholm, et al, Nature 365:566-568(1993) and references therein, including PNA clamps (Nucleic Acids Res. 23:217(1995)) and peptide-PNA conjugates including those disclosed by M. R. Lewis, et al, Bioconj Chem. 13, 1176 (2002) and references therein.

Also preferred nucleic acids are nucleotide mimics or co-oligomers like phosphoric acid ester nucleic acids (PHONA), disclosed by Peyman, et al., Angew. Chem. Int. Ed. Engl. 36:2809-2812 (1997). Also included are DNA and/or RNA, including any fragments or derivatives from viruses, bacteria, fungi and higher plants as well as from any tissue, cells, nuclei, chromosomes, cytoplasm, mitochondria, ribosomes, and other cellular sources.

Triplex-Forming Nucleic Acid.

A triplex-forming nucleic acid is a nucleic acid capable of forming a third, or triple strand with a specific DNA or RNA segment. Since the initial observation of triple-stranded DNA by Felsenfeld et al., J. Am. Chem. Soc. 79:2023 (1957), oligonucleotide-directed triple helix formation has emerged as a valuable tool in molecular biology. Current knowledge suggests that triplex-forming nucleic acids can bind as third strands of DNA in a sequence specific manner in the major groove in homopurine/homopyrimidine stretches in duplex DNA. In one motif, a homopyrimidine oligonucleotide binds in a direction parallel to the purine strand in the duplex, as described by Moser and Dervan, Science 238:645 (1987), Praseuth et al., Proc. Nad. Acad. Sci. USA 85:1349 (1988), and Mergny et al., Biochemistry 30:9791 (1991). In the alternate purine motif, a homopurine strand binds anti-parallel to the purine strand, as described by Beal and Dervan, Science 251:1360 (1991). Also preferred are any triplex-forming PNAs and triplex-forming backbone derivatized nucleic acids defined herein.

Mutagenic Triplex-Forming Nucleic Acid.

A mutagen is a category of chemicals capable of causing a mutation at the desired site of a double-stranded DNA molecule. Preferably the mutation restores the normal, functional sequence of the gene, inactivates an oncogene or activates an oncogene suppressor, or alters the function or inactivates a viral gene. Examples include radionuclides such as ¹²⁵I, ³⁵S and ³²P, and molecules become mutagenic with radiation, such as boron that interacts with neutron capture and iodine that interacts with auger electrons.

A mutagenic, triplex-forming nucleic acid is a mutagenic nucleic acid capable of forming a triple strand with a specific DNA or RNA segment and chemically modifying some portion of the segment. Generally, a mutagenic nucleic acid hybridizes to a chosen site in the target gene, forming a triplex region, thereby bringing the attached mutagen into proximity with the target gene and causing a mutation at a specific site in the gene.

If the target gene contains a mutation that is the cause of a genetic disorder, then a mutagenic oligonucleotide is useful in this invention for mutagenic repair that may restore the DNA sequence of the target gene to normal. If the target gene is a viral gene needed for viral survival or reproduction or an oncogene causing unregulated proliferation, such as in a cancer cell, then the mutagenic oligonucleotide is useful in this invention for causing a mutation that inactivates the gene to incapacitate or prevent reproduction of the virus or to terminate or reduce the uncontrolled proliferation of the cancer cell. A mutagenic oligonucleotide is also a useful anti-cancer agent in this invention for activating a repressor gene that has lost its ability to repress proliferation.

Targeting or Biorecognition Molecules.

For the purposes of this invention, targeting or biorecognition molecules are moieties suitable for pharmaceutical use that bind to the surface or biological site of a specific cell, tissue or organism. The biological site is considered the “target” of the biorecognition molecule or “targeting moiety” that binds to it. In the prior art, certain drugs are “targeted” by coupling them to a targeting molecule that has a specific binding affinity for the cells, tissue or organism that the drug is intended for. For targeting a composition of this invention, a targeting molecule is coupled to any suitable chloroquine substance that also has coupled a nucleic acid. Or, a targeting molecule is coupled to any suitable chloroquine that includes an active agent and a carrier substance coupled to it. Categories of targeting molecules useful in this invention are described below under “ligand”, “antibody” and “receptor”.

Ligand.

A ligand functions as a type of targeting or biorecognition molecule defined as a selectively bindable material that has a selective (or specific), affinity for another substance. The ligand is recognized and bound by a usually, but not necessarily, larger specific binding body or “binding partner”, or “receptor”. Examples of ligands suitable for targeting are antigens, haptens, biotin, biotin derivatives, lectins, galactosamine and fucosylamine moieties, receptors, substrates, coenzymes and cofactors among others.

When applied to this invention, a ligand includes an antigen or hapten that is capable of being bound by, or to, its corresponding antibody or fraction thereof. Also included are viral antigens, nucleocapsids and cell-binding viral derivatives including those from any DNA and RNA viruses, AIDS, HIV and hepatitis viruses, adenoviruses, adeno-associated viruses (AAV), alphaviruses, arenaviruses, coronaviruses, flaviviruses, herpesviruses, myxoviruses, oncornaviruses, papovaviruses, paramyxoviruses, parvoviruses, picornaviruses, poxviruses, reoviruses, rhabdoviruses, rhinoviruses, togaviruses and viroids; any bacterial antigens including those of gram-negative and gram-positive bacteria, acinetobacter, achromobacter, bacteroides, clostridium, chlamydia, enterobacteria, haemophilus, lactobacillus, neisseria, staphyloccus, and streptoccocus; any fungal antigens including those of aspergillus, candida, coccidiodes, mycoses, phycomycetes, and yeasts; any mycoplasma antigens; any rickettsial antigens; any protozoan antigens; any parasite antigens; any human antigens including those of blood cells, virus infected cells, genetic markers, heart diseases, oncoproteins, plasma proteins, complement factors, rheumatoid factors. Included are cancer and tumor antigens such as alpha-fetoproteins, prostate specific antigen (PSA) and CEA, cancer markers and oncoproteins, among others.

Other substances that can function as ligands for targeting are certain vitamins (i.e. folic acid, B₁₂), steroids, prostaglandins, carbohydrates, lipids, antibiotics, drugs, digoxins, pesticides, narcotics, neuro-transmitters, and substances used or modified such that they function as ligands.

Ligands also include various substances with selective affinity for receptors that are produced through recombinant DNA, genetic and molecular engineering. Except when stated otherwise, ligands of the instant invention also include the ligands as defined by K. E. Rubenstein, et al, U.S. Pat. No. 3,817,837 (1974).

Also included are any suitable vitamins for targeting such as vitamin B6 (T. Zhu, et al., (1994) Bioconjugate Chem. 5, 312.). Also included are targeting receptors such as for liver cells using the asialo-glycoprotein receptors (X. M. Lu, et al, (1994) Nucl. Med. 35, 269). Also included are suitable octreotides or octreotate, the carboxylic acid derivative of octreotide for targeting somatostatin receptors, among others. Also included are peptides which bind to integrins and the EGF receptor family.

Antibody.

When applied to targeting moieties of this invention, one category is an antibody, which is defined to include all classes of antibodies and monoclonal antibodies. Also included are antibodies used for specific cell or tissue targeting such as antibodies that bind to specific cell receptors such as anti-transferrin antibodies used to cross the blood brain barrier. Also included are monoclonal antibodies for targeting of nucleic acids including peptide nucleic acid (PNA) or other nucleic acids (i.e. W. M. Pardridge, et al (1995) Proc.Natl. Acad. Sci. U.S.A. 92, 5592.).

Synthetic Antibody.

Another category of targeting moieties is synthetic antibodies, defined as antibody derivatives or genetically engineered antibodies. These include chimeric antibodies, Fab fractions of antibodies, antibody fragments and derivatives thereof.

Receptor.

A receptor functions as a type of targeting molecule defined for this invention as a specific binding body or “partner” or “ligator” that is usually, but not necessarily, larger than the ligand it can bind to. For the purposes of this invention, it is a specific substance or material or chemical or “reactant” that is capable of selective affinity binding with a specific ligand.

Under certain conditions, the instant invention is also applicable to using other substances as receptors. For instance, other receptors suitable for targeting include naturally occurring receptors, any hemagglutinins and cell membrane that bind specifically to hormones, vitamins, drugs, antibiotics, cancer markers, genetic markers, viruses, and histocompatibility markers.

Other receptors also include enzymes, especially cell surface enzymes such as neuraminidases. Also included are chalones, cavitands, thyroglobulin, intrinsic factor, chelators, staphylococcal protein A, protein G, bacteriophages, cytochromes and lectins.

Most preferred are certain proteins or protein fragments (i.e. hormones, toxins), and synthetic or natural polypeptides with cell surface affinity such as growth factors that include basic fibroblast growth factors (bFGF). Preferred targeting molecules also include certain proteins and protein fragments or derivatives with affinity for the surface of any cells, tissues or microorganisms that are produced through recombinant DNA, genetic and molecular engineering.

Blood-Brain Barrier Agents.

An important and separate category of substances are blood-brain barrier (BBB) targeting agents. Blood-brain barrier agents are substances that can penetrate the BBB and carry other substances into the brain. There are certain compounds needed for penetrating the BBB, as are disclosed by D. J. Begley, in J. Pharm. Pharmacol. 48, 136-146 (1996) and by W. M. Partridge, et al, in J. Cereb. Blood Flow Metab. 17, 713-731 (1997), and incorporated herein. Such compounds include those which are more lipophilic, are capable of changing to effective chirality after crossing the blood-brain barrier, have side chain moieties which enhance compound transport via blood-brain barrier transporter mechanisms, or are coupled with specific BBB-penetrating antibodies. Such BBB-penetrating antibodies are limited to those with affinity to specific transferrin receptors of the BBB such as the lactotransferrin receptor in humans.

Transduction Vector.

Transduction vectors are known in the prior art under a wide variety of names. For this invention a transduction vector is defined as a peptide substance suitable for pharmaceutical use that promotes cellular uptake across the cell membrane and may include intracellular transport such as into the cell nucleus. Preferred transduction vectors or “fusion vectors” or “fusion moieties” or “membrane transduction” moieties are certain membrane translocation or membrane transfer peptides that can also include carbohydrates, lipids and polymers and combinations of these substances. Preferred transduction vectors are peptides (“fusion peptides” or “peptide vectors”) including those with “transduction domains” in their amino acid sequence.

Preferred transduction vectors have a molecular weight between 1000 and 100,000 Daltons, most preferred between 1200 and 80,000 Daltons. Transduction vectors as defined for this invention specifically exclude as unsuitable due to their antigenic potential, complex proteins such as antibodies and enzymes.

Some preferred transduction vectors for this invention include, but are not limited to, any derived sequences or extracts of any signal peptides or any fusogenic peptides including: TAT (i.e. from HIV virus), herpes simplex virus VP-22, hepatitis B virus PreS2 translocation motif (ITM) and antennapedia homeoproteins (i.e. penetratins). Preferred transduction vectors also include poly arginines (i.e. containing 5 or more, preferably from 6 to 12 arginines and with or without one or more terminal cysteines), poly histidines, poly lysines, poly ornithines and combinations of these amino acids with or without one or more terminal cysteines.

Also included are the peptide vectors disclosed by P. M. Fischer, et al, in Reviews Bioconj. Chem 12, 825-841 (2001) and references therein. Preferred examples of transduction vectors in this invention are peptide vectors which have been employed for transport of active agents including nucleic acids into cells. Preferred examples include conjugates of a carrier substance with penetratins or signal peptides to increased uptake rates due to the membrane translocation properties of these peptides. Table I. is a list of some peptides that are preferred transduction vectors in this invention.

TABLE I

| | NAME (origin of |
|---|---|
| TRANSDUCTION VECTOR SEQUENCE | sequence) |
| RQIKIWFQNRRMKWKK | pAntp(43-58); Pene- |
| | tratin |
| KKWKMRRNQFWVKVQR | retro-inverso |
| | pAntp(43-58) |
| RRWRRWWRRWWRRWRR | W/R Penetratin |
| RQIKIWFQNRRMKWKKEN 24 | antennapedia pep- |
| | tide |
| RRMKWKK | pAntp(52-58) |
| GRKKRRQRRRPPQ | HIV TAT |
| YGRKKRRQRRR | HIV TAT |
| PTSQSRGDPTGPKE | HIV TAT C-terminus |
| | peptide |
| AVGAIGALFLGFLGAAG | viral fusion pep- |
| | tide |
| GALFLGWLGAAGSTMGA | gp41 fusion se- |
| | quence |
| GALFLGFLGAAGSTMGAWSQPKSKRKV MPG | (gp41 fusion se- |
| | quence SV40 NLS) |
| DRVIEVVQGAYRAIRNIPRRIRQG | CR-gp41 fusion pep- |
| | tide |
| MGLGLHLLVLAAALQGA | C. crocodylus Ig(v) |
| | light chain |
| MGLGLHLLVLAAALQGAWSQPKKKRKV | C. crocodylus Ig(v) |
| | light chain - SV40 |
| | NLS |
| PLSSIFSRIGDP | PreS2-TLM |
| GWTLNSAGYLLGKINLKALAALAKKIL | Transportan |
| RGGRLSYSRRRFSTSTGR | SynB1 |
| AAVALLPAVLLALLAP | MPS (kaposi FGF |
| | signal sequence) |
| AAVLLPVLLAAP | MPS (kaposi FGF |
| | signal sequence) |
| VTVLALGALAGVGVG | MPS (human integrin |
| | beta3 signal seq) |
| VAYISRGGVSTYYSDTVKGRFTRQKYNKRA | P3 |
| KLALKLALKALKAALKLA | Model amphiphilic |
| | peptide |
| WEAKLAKALAKALAKHLAKALAKALKACEA | KALA |
| GLFEAIAGFIENGWEGMIDGGGYC | hemagglutinin enve- |
| | lope fusion peptide |
| RRRRRRR | R7 |
| AAVALLPAVLLALLAPVQRKRQKLMP | engineered |
| MGLGLHLLVLAAALQGAKKKRKV | engineered |

Cell Receptor Binding Peptides.

Also preferred are known cell receptor binding peptides that bind to distinct receptors, which upon binding, mediate endocytosis of a peptide-ODN complex. Also included are peptides which bind to integrins and to the EGF receptor family. Table II. is a list of some receptor binding peptides that are preferred in this invention:

TABLE II

| RECEPTOR BINDING PEP- | |
|---|---|
| TIDE SEQUENCE | NAME (function) |
| TQPREEQYNSTFRV | Fc receptor binding peptide |
| D-GCSKAPKLPAALC | antagonist to IGF-1 receptor |
| YGGFLRRG | beta-endophin receptor ligand |
| YEE(ah-GalNAc)3 | hepatocyte specific delivery |
| Z-D-Phe-L-Phe-Gly | cell fusion and hemolysis |
| | inhibitor |

Cyclodextrin

A cyclodextrin (CD) monomer, is an oligosaccharide composed of glucose molecules coupled together to form a ring that is conical with a hydrophobic, hollow interior or cavity. Cyclodextrin monomers are one of the starting materials for making grafted polymers as described in the instant invention. They can be any cyclodextrin suitable for pharmaceutical use, including alpha-, beta-, and gamma-cyclodextrins, and their combinations, analogs, isomers, and derivatives.

In describing this invention, references to a cyclodextrin “complex”, means a noncovalent inclusion complex. An inclusion complex is defined herein as a cyclodextrin functioning as a “host” molecule, combined with one or more “guest” molecules that are contained or bound, wholly or partially, within the hydrophobic cavity of the cyclodextrin or its derivative.

Cyclodextrin Dimers, Trimers and Polymers.

For this invention, a cyclodextrin dimer is a preferred category of cyclodextrin derivative defined as two cyclodextrin molecules covalently coupled or cross-linked together to enable cooperative complexing with a guest molecule. Examples of some CD dimers that can be derivatized and used in the drug carriers of this invention, are described by; Breslow, R., et al, Amer. Chem. Soc. 111, 8296-8297 (1989); Breslow, R., et al, Amer. Chem. Soc. 105, 1390 (1983) and Fujita, K., et al, J. Chem. Soc., Chem. Commun., 1277 (1984).

A cyclodextrin trimer is another preferred category of cyclodextrin derivative defined as three cyclodextrin molecules covalently coupled or cross-linked together to enable cooperative complexing with a guest molecule. Another preferred cyclodextrin is a cyclodextrin polymer defined as a unit of more than three cyclodextrin molecules covalendy coupled or cross-linked together to enable cooperative complexing with several guest molecules. Also included are the “linear” cyclodextrin polymers disclosed by Davis, et al, U.S. Pat. No. 6,509,323 B1.

For this invention, preferred cyclodextrin dimer, trimer and polymer units are synthesized by covalendy coupling through chemical groups such as through coupling agents. The synthesis of preferred cyclodextrin dimer, trimer and polymer units does not include the use of proteins or other “intermediate coupling substances”. Cooperative complexing means that in situations where the guest molecule is large enough, the member cyclodextrins of the CD dimer, trimer or polymer can each noncovalently complex with different parts of the same guest molecule, or with smaller guests, alternately complex with the same guest.

The prior art has disclosed dimers and polymers comprised of cyclodextrins of the same size. An improved cyclodextrin dimer, trimer or polymer comprises combinations of different sized cyclodextrins to synthesize these units. These combinations may more effectively complex with guest molecules that have heterogeneous complexing sites. Combinations for this invention can include the covalent coupling of an alpha CD with a beta CD, an alpha CD with a gamma CD, a beta CD with a gamma CD and polymers with various ratios of alpha, beta and gamma cyclodextrins.

Most preferred are cyclodextrin dimers, trimers and polymers containing cyclodextrin derivatives such as carboxymethyl CD, glucosyl CD, maltosyl CD, hydroxypropyl cyclodextrins (HPCD), 2-hydroxypropyl cyclodextrins, 2,3-dihydroxypropyl cyclodextrins (DHPCD), sulfobutylether cyclodextrins (SBECD), ethylated and methylated cyclodextrins.

Also preferred are oxidized cyclodextrin dimers, trimers and polymers that provide aldehydes and any oxidized derivatives that provide aldehydes. Some examples of suitable derivatives are disclosed by Pitha, J., et al, J. Pharm. Sci. 75, 165-167 (1986) and Pitha, J., et al, Int. J. Pharmaceut. 29, 73-82 (1986).

Also preferred are any amphiphilic CD dimers, trimers and polymers made from derivatives such as those disclosed by K. Chmurski, et al., Langmuir 12, 4046 (1996), P. Zhang, et al., J. Phys. Org. Chem. 5, 518 (1992), M. Weisser, et al., J. Phys. Chem. 100, 17893 (1996), L. A. Godinez, et al., Langmuir 14, 137 (1998) and D. Duchene, “International Pharmaceut. Applic. of Cyclodextrins Conference”, Lawrence, Kans., USA, Jun. 1997, and references therein.

Also included are altered forms, such as crown ether-like compounds prepared by Kandra, L., et al, J. Inclus. Phenom. 2, 869-875 (1984), and higher homologues of cyclodextrins, such as those prepared by Pulley, et al, Biochem. Biophys. Res. Comm. 5, 11 (1961). Some useful reviews on cyclodextrins are: Atwood J. E. D., et al, Eds., “Inclusion Compounds”, vols. 2 & 3, Academic Press, NY (1984); Bender, M. L., et al, “Cyclodextrin Chemistry”, Springer-Verlag, Berlin, (1978) and Szejtli, J., “Cyclodextrins and Their Inclusion Complexes”, Akademiai Kiado, Budapest, Hungary (1982). These references, including references contained therein, are applicable to the synthesis of the preparations and components of the instant invention and are hereby incorporated herein by reference.

Cyclodextrin Blocks.

A CD-block is a category of carrier substances defined as a CD dimer, trimer or polymer that is used as a component, or unit (i.e. building block) for additional cross linking with other polymer blocks to produce a carrier substance suitable for pharmaceutical use or are coupled to the carrier substances of this invention.

Preferred cyclodextrin blocks (CD block) are compositions that provide for the incorporation of cyclodextrin derivatives into carrier substances that include micelle-forming amphiphilic molecules through copolymerization with other polymer blocks or grafted polymers defined herein. The CD blocks can include CD dimers, CD trimers or CD polymers. The CD blocks can be primarily hydrophilic to produce micelles with the CD moieties in the hydrophilic shell. Or, the CD blocks can be primarily hydrophobic to produce micelles with the CD moieties in the hydrophobic core.

The CD blocks also have available suitable reactive groups that can copolymerize with other block polymers, using suitably modified methods described and referenced by G. S. Kwon, IN: Critical Reviews in Therapeutic drug Carrier Systems, 15(5):481-512 (1998).

For example, a CD derivative (i.e. CD dimer) is prepared and made hydrophobic by adding alkyl or aromatic groups (i.e. methylation, ethylation, or benzylation), and also has available an N carboxyanhydride (NCA) group coupled through a suitable spacer.

One form of CD block would be methylated-CD-CD-poly(aspartate)_{N}-NCA (where N=1-10). This CD block can then be copolymerized with suitable blocks of alpha-methyl-omega-amino-polyethylene oxide) (PEO) in suitable solvent (CHCl₃:DMF) to produce a micelle-forming diblock amphiphilic molecule. The resulting diblock is CD-block-PEO. With suitable modifications PEG can be used in place of PEO. Also, triblocks such as PEO-block-CD-block-PEO can be prepared and used.

Other combinations for the CD-blocks of this invention can include the covalent coupling of an alpha CD with a beta CD, an alpha CD with a gamma CD, a beta CD with a gamma CD and polymers with various ratios of alpha, beta and gamma cyclodextrins.

Grafted Polymer.

A grafted polymer is a category of carrier substances defined as polymeric substances suitable for pharmaceutical use including copolymers and block polymers that are suitably coupled to produce a carrier substance as defined in the present invention. Preferably grafted polymers are biocompatible and generally hydrophilic. Preferred grafted polymers include any polyethylene glycols (PEG), methoxy polyethylene glycols (mPEG), poly(ethylene-co-vinyl acetate) (EVAc), N-(2-hydroxypropyl)methacrylamide polymer (HPMA), poly(2-(dimethyl amino)ethyl methacrylate (DMAEMA), poly(D, L-lactide-co-glycolide) (PLGA), poly(polypropyl acrylic acid) (PPAA), polyethylenimine (PEI), polyamidoamines (PAMAM), polylysine (PLL), poly-L-histidines (PLH), CD dimers, CD trimers, CD polymers and CD blocks, defined herein. Preferably grafted polymers also include any suitable combination of the polymers defined herein. Also preferred are comb shaped polymers including N-Ac-poly(L-histidine)-graft-poly(L-lysine) disclosed by J. M. Benns, et al, in Bioconj. Chem. 11, 637-645 (2000), and references therein that are incorporated herein. Grafted polymers are included wherein said grafted polymer is appropriately endcapped as is known in the prior art and which also may be substituted with moieties that do not adversely affect the functionality of the grafted polymer for its intended purpose. The CD-grafted polymers of the present invention can be synthesized by coupling two to thirty CDs or derivatives thereof to a carrier substance.

Liposome.

A liposome or vesicle is defined as a water soluble or colloidal structure composed of amphiphilic molecules that have formed generally spherical bilayer membranes. Said amphiphilic molecules are generally oriented in said bilayer membrane so that their hydrophilic ends are on the outside of the membrane and their hydrophobic ends are sequestered inside the membrane. Preferred liposomes of this invention generally have a spherical shape where said bilayer membranes are arranged in one or more layers (lamella) around a single, primarily hydrophilic or aqueous, central zone. Unilamellar liposomes have one bilayer membrane surrounding a central hydrophilic zone. Multilamellar liposomes have more than one surrounding membrane with hydrophilic zones between said membranes that surround the central hydrophilic zone.

Liposomes can be composed of any suitable amphiphilic molecules described herein. Also, the amphiphilic molecules can be suitably polymerized or cross linked, including the use of biocleavable linkages.

Micelles and Nanoparticles.

A preferred micelle or nanoparticle for this invention is defined as a water soluble or colloidal structure or aggregate (also called a nanosphere) composed of one or more amphiphilic molecules and may include grafted polymers defined herein. Preferred micelles and nanoparticles of this invention generally have a single, central and primarily hydrophobic zone or “core” surrounded by a hydrophilic layer or “shell”. This shape may also be due to aggregation and/or condensation of the carrier due to self attraction.

Preferred micelles for use as carrier substances in this invention include those disclosed in U.S. patent application Ser. No. 09/829,551, filed Apr. 10, 2001, the contents of which are incorporated herein.

Also preferred are nanoparticles composed of macromolecules including “cascade polymers” such as dendrimers. Preferred dendrimers include polyamidoamines as disclosed by J. Haensler, et al, in Bioconj. Chem. 4, 372-379 (1993) and references therein.

Micelles and nanoparticles range in size from 5 to about 2000 nanometers, preferably from 10 to 400 nm. Micelles and nanoparticles of this invention are distinguished from and exclude liposomes which are composed of bilayers.

The micelles of this invention can be composed of either a single monomolecular polymer containing hydrophobic and hydrophilic moieties or an aggregate mixture containing many amphiphilic (i.e. surfactant) molecules formed at or above the critical micelle concentration (CMC), in a polar (i.e. aqueous) solution.

Pendant PEG.

Pendant polyethylene glycol is one preferred carrier substance for synthesizing the compositions of the present invention suitable for pharmaceutical use. Pendant PEG is defined here as derivatized or “grafted” with side functional groups or “branches” along the backbone of the molecule. The functional groups are frequently propionic acid groups comprising a three carbon alkyl side chain with a terminal carboxylic acid. However, the grafted functional side group can be comprised of alkyl chains of 2, 3, 4, 5, 6, or more carbon atoms that terminate in carboxylic acid, or a primary amine, or an aldehyde, or a thiol, or combinations of these.

Pendant PEG (also called “multi-branched PEG”), is commercially available in a variety of molecular masses and with various numbers of functional groups per molecule. For instance, SunBio USA, Orinda, Calif. 94563, offers such material in molecular weights of 10, 12, 18, 20, 30, 35 and 100 kilo Daltons (KDa) and with 6, 8, 10, 12, 14, 16, 18, or 20 functional side groups or “branches” per molecule.

For the present invention, preferred pendant PEG starting material ranges from 6,000 Daltons to 100,000 Daltons, most preferably a molecular weight of 20,000 or greater to prevent rapid elimination of the PEG-conjugated composition from the bloodstream.

In one preferred embodiment of the present invention, PEG containing carboxyl groups (20,000 Daltons or 25,000 Daltons containing 8 to 15 carboxyl groups per PEG molecule) is used as the starting material to conjugate with the chloroquines and active agents including nucleic acids. In order to keep the steric hindrance effect to a minimum, a flexible linear linkage may be used to keep the nucleic acid moiety away from the polymer backbone. Due to the biocompatibility of the materials and pliability of the polymers of the present invention, they will cause minimal nonspecific toxicity.

Targeted Chloroquine-Coupled Carriers.

A targeted chloroquine-coupled carrier is composed of a carrier substance suitable for pharmaceutical use that has chloroquines and a targeting molecule coupled to it. The carrier is thereby targeted through the specific binding properties of the targeting molecule coupled to the surface. During the coupling of the targeting molecule, the functions of the targeting molecule, chloroquines and the targeted carrier are not irreversibly or adversely inhibited. Preferably, the targeting molecule maintains specific binding properties that are functionally identical or homologous to those it had before coupling. Preferably, the targeting molecule is coupled through a suitable spacer to avoid steric hindrance.

Targeted carriers coupled to avidin and streptavidin are useful for subsequent noncovalent coupling to any suitable biotinylated chloroquine substance and nucleic acid. Similarly, chloroquines and nucleic acid suitably coupled to antibody can be noncovalently (antigenically) coupled to another antibody, or to a peptide or other suitable substance that has the appropriate biorecognition properties. Another useful composition comprises protein A, protein G, or any suitable lectin that has been covalently coupled to chloroquines and nucleic acids of this invention.

Capping Moiety.

A capping moiety is defined here as a substance suitable for pharmaceutical use that is used to consume or cap any available reactive groups or functional groups to prevent further coupling or other reactions on the carrier of this invention. The capping moiety may also provide certain desired properties such as neutral charge, or positive charge or negative charge as desired. The capping moiety may also provide increased water solubility or may provide hydrophobicity. The capping moiety may also provide a type of label for colorimetric or fluorometric detection.

Some preferred examples of capping moieties are ethanol amines, glucose amines, mercaptoethanol, any suitable amino acids, including gylcines, alanines, leucines, phenylalanines, serines, tyrosines, tryptophanes, asparagines, glutamic acids, cysteines, lysines, arginines and histidines, among others. Preferred capping moieties also include suitable halogens (including Br, Cl, I, and F) and fluorophores or dyes.

1. Pendant PEG Nucleic Acid Carrier.

One preferred nucleic acid carrier is defined as a pendant PEG polymer backbone wherein nucleic acid moieties are covalently coupled to said pendant PEG through branched functionalities on the backbone. The polymeric composition of this invention is a mixture of polymer units where the number of units in the polymer may be variable and the number of nucleic acid moieties may vary. Hence, each polymer has an average molecular weight and an average number of nucleic acids per polymer backbone within such polymeric composition. Said pendant polyethylene glycol polymer backbone has a molecular weight range from 2,000 to 1,000,000 Daltons, preferably 5,000 to 70,000 Daltons, and most preferably 20,000 to 40,000 Daltons.

Accordingly, the nucleic acid loaded, pendant polyethylene glycol carrier of the present invention can be represented by the following formula:

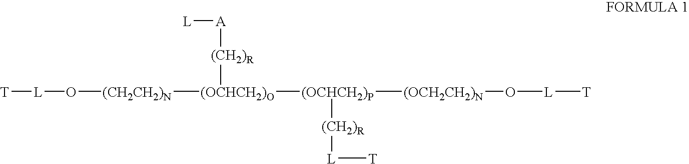

Formula 1 represents a horizontal polyethylene glycol backbone comprising connected units; (OCH₂CH₂)_{N}, (OCHCH₂)_{O} and (OCHCH₂)_{P}; which may alternate in their number, sequence and frequency within the polymer backbone.

For said connected units, N and O are independent integers equaling average values between 1 and 30 of their respective units. P is an independent integer equaling an average value between 0 (zero) and 30 of its respective unit, meaning that if P=zero there are no respective units for P in the backbone. Also, wherein a mixture of different N, O and P values may be found for their respective units.

The polymer backbone also includes the branching or pendant unit; (CH₂)_{R} covalently coupled to said PEG backbone and wherein R is an integer between 1 and 30, preferably between 2 and 10. Also, wherein said pendant unit terminates in either a functional group or is terminally coupled to moieties “L-A” or “L-T” as defined below.

In Formula 1, A is a nucleic acid as disclosed herein independently and covalendy coupled to the pendant polyethylene glycol backbone through linkage L.

In Formula 1, T is independently and covalendy coupled to the pendant polyethylene glycol backbone through linkage L.

T is a member independently selected from the group consisting of hydrogen (H), hydroxyl (OH), halogen, chloroquine substance, targeting moiety (TM), transduction vector (TV), amphiphilic molecule and capping moiety.

T may also be a member independently selected from the group consisting of a grafted polymer as disclosed herein that is biocompatible and includes PEG, HPMA, PEI, PLL, CD, CD dimers, CD trimers and CD polymers. Wherein said grafted polymer is appropriately endcapped as is known in the prior art and which also may be substituted with substituents that do not adversely affect the functionality of the grafted polymer for its intended purpose.

Also wherein said grafted polymer has a molecular weight range from 500 to 100,000 Daltons, preferably from 1,000 to 10,000 Daltons.

Also wherein T as described herein is coupled to said pendant polyethylene glycol backbone with the proviso that a mixture of hydrogens, hydroxyls, chloroquine substances, targeting moieties, cell transduction vectors, amphiphilic molecules and grafted polymers may be found on the same polyethylene glycol backbone and/or within the same polyethylene glycol polymer composition.

L is a covalent linkage between said polyethylene glycol and nucleic acid intercalator A or T as defined herein, through functional groups defined herein and may include one or more coupling agents as defined herein. Said linkage L may also include suitable spacer molecules and may be a biocleavable linkage as defined herein.

In another preferred embodiment of this invention Formula 1 is a chloroquine-coupled carrier wherein T is at least one moiety selected from the group of chloroquine substances as described herein.

2. Chloroquine-Coupled Pendant PEG Nucleic Acid Carrier With Intercalator Coupling.

A preferred chloroquine-coupled carrier is a pendant PEG polymer backbone as defined herein, wherein intercalator moieties are also covalendy coupled to said pendant PEG through branched functionalities on the backbone. Instead of directly coupling nucleic acids, said nucleic acids are subsequently “loaded” onto the carrier by coupling them to the carrier through intercalation with said intercalators on the carrier.

Accordingly, the unloaded, chloroquine-coupled pendant PEG carrier of the present invention, before coupling to nucleic acid, can be represented by the following formula:

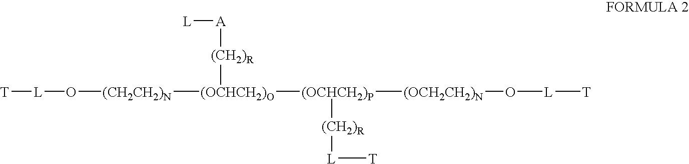

Formula 2 represents a horizontal polyethylene glycol backbone as is described in Formula 1, comprising connected units; (OCH₂CH₂)_{N}, (OCHCH₂)_{O} and (OCHCH₂)_{P}; which may alternate in their number, sequence and frequency within the polymer backbone.

The polymer backbone also includes the branching or pendant unit; (CH₂)_{R} covalently coupled to said PEG backbone and wherein R is an integer between 1 and 30, preferably between 2 and 10. Also, wherein said pendant unit terminates in either a functional group or is terminally coupled to moieties “L-A” or “L-T” as defined below.

In Formula 2, A is an intercalator as disclosed herein independently and covalendy coupled to the pendant polyethylene glycol backbone through linkage L.

In Formula 2, T is independently and covalendy coupled to the pendant polyethylene glycol backbone through linkage L.

In a preferred embodiment of this invention, T is at least one moiety selected from the group of chloroquine substances as described herein.

In addition to a chloroquine substance, T may also be a member independently selected from the group consisting of hydrogen (H), hydroxyl (OH), halogen, targeting moiety (TM), transduction vector (TV), amphiphilic molecule and capping moiety.

In addition to a chloroquine substance, T may also be a member independently selected from the group consisting of a grafted polymer as disclosed herein that is biocompatible and includes PEG, HPMA, PEI, PLL, CD, CD dimers, CD trimers and CD polymers. Wherein said grafted polymer is appropriately end capped as is known in the prior art and which also may be substituted with moieties that do not adversely affect the functionality of the grafted polymer for its intended purpose. Also wherein said grafted polymer has a molecular weight range from 500 to 100,000 Daltons, preferably from 1,000 to 10,000 Daltons.

Also wherein T as described herein is coupled to said pendant polyethylene glycol backbone with the proviso that a mixture of chloroquine substances, hydrogens, hydroxyls, targeting moieties, cell transduction vectors, amphiphilic molecules and grafted polymers may be found on the same polyethylene glycol backbone and/or within the same polyethylene glycol polymer composition.

L is a covalent linkage between said polyethylene glycol and nucleic acid intercalator A or T as defined herein, through functional groups defined herein and may include one or more coupling agents as defined herein. Said linkage L may also include suitable spacer molecules and may be biocleavable as defined herein.

3. Chloroquine-Coupled Carrier Substance.

A preferred chloroquine-coupled carrier substance is a carrier substance as defined herein, containing one or more chloroquine substances covalently coupled to said carrier substance.

Accordingly, the chloroquine-coupled carrier substance of the present invention can be represented by the following formula:

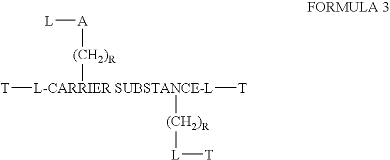

Formula 3 represents any suitable carrier substance as defined herein that includes a chloroquine substance and coupled moieties as described below. The carrier substance also includes one, two or mote branching or pendant units; (CH₂)_{R} covalently coupled to said carrier substance and wherein R is an integer between 1 and 30, preferably between 2 and 10. Also, wherein said pendant unit terminates in either a functional group or is terminally coupled to moieties “L-A” or “L-T” as defined below and which said units may alternate in their number, sequence and frequency depending on the desired carrier substance used.

In Formula 3, A is at least one moiety selected from the group of nucleic acids as disclosed herein independently and covalently coupled to the carrier substance through linkage L.

In Formula 3, T is independently and covalently coupled to the carrier substance through linkage L.

In a preferred embodiment of this invention, T is at least one moiety selected from the group of chloroquine substances as described herein.

In addition to a chloroquine substance, T may also be a member independently selected from the group consisting of hydrogen (H), hydroxyl (OH), halogen, targeting moiety (TM), transduction vector (TV), amphiphilic molecule and capping moiety.

In addition to a chloroquine substance, T may also be a member independently selected from the group consisting of a grafted polymer as disclosed herein that is biocompatible and includes PEG, HPMA, PEI, PLL, CD, CD dimers, CD trimers and CD polymers. Wherein said grafted polymer is appropriately end capped as is known in the prior art and which also may be substituted with moieties that do not adversely affect the functionality of the grafted polymer for its intended purpose. Also wherein said grafted polymer has a molecular weight range from 500 to 100,000 Daltons, preferably from 1,000 to 10,000 Daltons.

Also wherein T as described herein is coupled to said pendant polyethylene glycol backbone with the proviso that a mixture of chloroquine substances, hydrogens, hydroxyls, targeting moieties, cell transduction vectors, amphiphilic molecules and grafted polymers may be found on the same polyethylene glycol backbone and/or within the same polyethylene glycol polymer composition.

L is a covalent linkage between said polyethylene glycol and nucleic acid intercalator A or T as defined herein, through functional groups defined herein and may include one or more coupling agents as defined herein. Said linkage L may also include suitable spacer molecules and may be biocleavable as defined herein.

4. Chloroquine-Coupled Nucleic Acid Carrier Substance With Intercalator Coupling.

A preferred chloroquine-coupled carrier substance is a carrier substance as defined herein, containing one or more chloroquine substances covalently coupled to said carrier substance and wherein intercalator moieties are also covalently coupled to said carrier substance. Instead of directly coupling nucleic acids, said nucleic acids are subsequently “loaded” onto the carrier by coupling them to the carrier through intercalation with said intercalators on the carrier.

Accordingly, the unloaded, chloroquine-coupled carrier substance of the present invention, before coupling to nucleic acid, can be represented by the following formula:

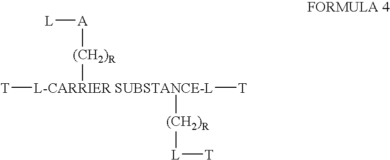

Formula 4 represents any suitable carrier substance as defined herein that includes a chloroquine substance and coupled moieties as described below. The carrier substance also includes one, two or more branching or pendant units; (CH₂)_{R} covalently coupled to said carrier substance and wherein R is an integer between 1 and 30, preferably between 2 and 10. Also, wherein said pendant unit terminates in either a functional group or is terminally coupled to moieties “L-A” or “L-T” as defined below and which said units may alternate in their number, sequence and frequency depending on the desired carrier substance used.

In Formula 4, A is an intercalator as disclosed herein independently and covalently coupled to the carrier substance through linkage L.

In Formula 4, T is independently and covalently coupled to the carrier substance through linkage L.

In a preferred embodiment of this invention, T is at least one moiety selected from the group of chloroquine substances as described herein.

In addition to a chloroquine substance, T may also be a member independently selected from the group consisting of hydrogen (H), hydroxyl (OH), halogen, targeting moiety (TM), transduction vector (TV), amphiphilic molecule and capping moiety.

In addition to a chloroquine substance, T may also be a member independently selected from the group consisting of a grafted polymer as disclosed herein that is biocompatible and includes PEG, HPMA, PEI, PLL, CD, CD dimers, CD trimers and CD polymers. Wherein said grafted polymer is appropriately end capped as is known in the prior art and which also may be substituted with moieties that do not adversely affect the functionality of the grafted polymer for its intended purpose. Also wherein said grafted polymer has a molecular weight range from 500 to 100,000 Daltons, preferably from 1,000 to 10,000 Daltons.

Also wherein T as described herein is coupled to said pendant polyethylene glycol backbone with the proviso that a mixture of chloroquine substances, hydrogens, hydroxyls, targeting moieties, cell transduction vectors, amphiphilic molecules and grafted polymers may be found on the same polyethylene glycol backbone and/or within the same polyethylene glycol polymer composition.

L is a covalent linkage between said polyethylene glycol and nucleic acid intercalator A or T as defined herein, through functional groups defined herein and may include one or more coupling agents as defined herein. Said linkage L may also include suitable spacer molecules and may be biocleavable as defined herein.

### EXAMPLES OF THE BEST MODES FOR CARRYING OUT THE INVENTION

In the examples to follow, percentages are by weight unless indicated otherwise. During the synthesis of the compositions of the instant invention, it will be understood by those skilled in the art of organic synthesis, that there are certain limitations and conditions as to what compositions will comprise a polymer carrier suitable for pharmaceutical use and may therefore be prepared mutatis mutandis. It will also be understood in the art of chloroquines and nucleic acids that there are limitations as to which derivatives and/or coupling agents can be used to fulfill their intended function.

The terms “suitable” and “appropriate” refer to synthesis methods known to those skilled in the art that are needed to perform the described reaction or other procedure. In the references to follow, the methods are hereby incorporated herein by reference. For example, organic synthesis reactions, including cited references therein, that can be useful in the instant invention are described in “The Merck Index”, 9, pages ONR-1 to ONR-98, Merck & Co., Rahway, N.J. (1976), and suitable protective methods are described by T. W. Greene, “Protective Groups in Organic Synthesis”, Wiley-Interscience, NY (1981), among others. For synthesis of nucleic acid probes, sequencing and hybridization methods, see “Molecular Cloning”, 2nd edition, T. Maniatis, et al, Eds., Cold Spring Harbor Lab., Cold Spring Harbor, N.Y. (1989).

All reagents and substances listed, unless noted otherwise, are commercially available from Applied Biosystems Division, Perkin-Elmer; Aldrich Chemical Co., WI 53233; Sigma Chemical Co., Mo. 63178; Pierce Chemical Co., IL. 61105; Eastman Kodak Co., Rochester, N.Y.; Pharmatec Inc., Alachua Fla. 32615; and Research Organics, Cleveland, Ohio. Or, the substances are available or can be synthesized through referenced methods, including “The Merck Index”, 9, Merck & Co., Rahway, N.J. (1976). Additional references cited in U.S. Pat. No. 6,048,736 and PCT/US99/30820, are hereby incorporated herein by reference.

### Nucleic Acid Carriers.

The general synthesis approach is; (1) produce or modify or protect, as needed, one or more functional groups on a chloroquine substance and (2) using one or more coupling methods, couple a chloroquine substance to a nucleic acid directly or through a carrier substance suitable for pharmaceutical use.

Also, as described below, the carrier may be suitably derivatized to include other useful substances and/or chemical groups (e.g. targeting molecules), to perform a particular function. Depending on the requirements for chemical synthesis, the derivatization can be done before coupling the chloroquine substance, or afterward, using suitable protection and deprotection methods as needed.

The carrier substance can be suitably derivatized and coupled through well-known procedures used for available amino, sulfhydryl, hydroxyl, or vinyl groups. Also, for certain carbohydrates added to the carrier substance, vicinal hydroxyl groups can be appropriately oxidized to produce aldehydes. Any functional group can be suitably added through well-known methods while preserving the carrier substance structure and properties. Examples are: amidation, esterification, acylation, N-alkylation, allylation, ethynylation, oxidation, halogenation, hydrolysis, reactions with anhydrides, or hydrazines and other amines, including the formation of acetals, aldehydes, amides, imides, carbonyls, esters, isopropylidenes, nitrenes, osazones, oximes, propargyls, sulfonates, sulfonyls, sulfonamides, nitrates, carbonates, metal salts, hydrazones, glycosones, mercaptals, and suitable combinations of these. The functional groups are then available for suitable coupling or cross-linking using a bifunctional reagent.

Suitable coupling or cross-linking agents for preparing the carriers of the instant invention can be a variety of coupling reagents, including oxiranes (epoxides) previously described. Also useful are methods employing acrylic esters such as m-nitrophenyl acrylates, and hexamethylene diamine and p-xylylenediamine complexes, and aldehydes, ketones, alkyl halides, acyl halides, silicon halides and isothiocyanates.

Synthesis of Nucleic Acids With Suitable Functional Groups.

Because conventional automated synthesis of nucleic acids proceeds from 3′ to 5′, the 5′-terminus is readily available for the addition of functional groups. A general approach to the modification of the 5′-terminus is to use reagents which couple to the 5′-hydroxyl of an oligonucleotide. In this invention, the phosphoramidite reagents used include chloroquine substance phosphoramidites as described herein, and those that are compatible with automated DNA synthesizers. Many of these reagents are available from Glen Research Corp., Sterling, Va., and other suppliers.

5′-Modified Nucleic Acids.

A preferred group of phosphoramidite reagents is the 5′-Amino-Modifiers. The 5′-Amino-Modifiers are preferably for use in automated synthesizers to functionalize the 5′-terminus of a target oligonucleotide. The primary amine can be used to attach a variety of functional moieties to the oligonucleotide.

The 5′-Amino-Modifiers include 6-(4-Monomethoxytritylamino) hexyl-(2-cyanoethyl)-(N,N-diisopropyl)-phosphoramidite, M.W.: 589.76; 12-(4-Monomethoxytritylamino)dodecyl-(2-cyanoethyl)-(N,N-diisopropyl)-phosphoramidite, M.W.: 673.92 and 2-[2-(4-Monomethoxytrityl)aminoethoxy]ethyl-(2-cyanoethyl)-(N,N-diisopropyl)-phosphoramidite, M.W.: 577.71.

Also included are 6-(Trifluoroacetylamino)propyl-(2-cyanoethyl)-(N,Ndiisopropyl)-phosphoramidite, M.W.: 371.34 and 6-(Trifluoroacetylamino)hexyl-(2-cyanoethyl)-(N,N-diisopropyl)-phosphoramidite, M.W.: 413.42.

Another group of preferred reagents for adding an amino group are 5′-amino-modifiers such as β-cyanoethyl (CE) phosphoramidites which, when activated with 1H-tetrazole, can couple to the 5′-terminus of the nucleic acid with similar efficiency as nucleoside phosphoramidites.

5′-Thiol Nucleic Acid.

The phosphorothioate nucleic acids are synthesized using beta-cyanoethyl phosphoramidite chemistry. Acetylation is performed by 0.1 M acetic anhydride/tetrahydrofuran (THF) and 0.1 M imidazole/THF. Sulfurization is done by using the EDITH reagent.

The commercially available six-carbon thiol linker phosphoramidite (1-O-dimethoxytrityl-hexyl-disulfide-1′-[(2-cyanoethyl)-(N,N-diisopropyl)]-phosphoramidite (Glen Research) is coupled to the 5′ end. The final coupling is followed by an acetonitrile wash. The resin is dried under a stream of argon and treated with concentrated ammonia containing 0.1 M DTT at 55° C. for 12 h to simultaneously affect deprotection of the thiol protection as well as cleavage from the resin. The resin is removed by filtration and rinsed with concentrated ammonia. Evaporation of the resultant solution affords a clear residue which is dissolved in sterile water. To remove excess DTT, the solution is passed through a NAP-10 gel filtration column. The fractions containing the nucleic acid are immediately used for conjugation to chloroquines or to the chloroquine-coupled carrier.

Another preferred phosphoramidite reagent for adding a thiol functional group includes (S-Trityl-6-mercaptohexyl)-(2-cyanoethyl)-(N,N-diisopropyl)-Phosphoramidite M.W.: 576.78, which produces a thiol group at the 5′-terminus of a synthetic oligonucleotide or nucleic acid. Alternatively, coupling to the 3′-terminus, it is added to any suitable support and then the desired nucleic acid is synthesized. DTT is used during deprotection or after purification of the product nucleic acid to cleave the disulfide linkage.

A. Kumar, et al, in Nucleic Acids Res., 19, 4561 (1991) describes a procedure useful in this invention to modify a 5′-amino-modified oligonucleotide to a thiol using N-acetyl-DL-homocystein thiolactone. Another preferred group includes those designed to introduce a thiol group to the 3′-terminus of a target oligonucleotide such as 1-O-Dimethoxytrityl-propyl-disulfide, 1′-succinoyl-long chain alkylamino-CPG.

Another preferred group of phosphoramidite reagents in this invention includes spacer phosphoramidites such as 9-O-Dimethoxytrityl-triethyleneglycol, 1-[(2-cyanoethyl)-(N,N-diisopropyl)]-phosphoramidite, M.W.: 652.77, 18-O-Dimethoxytrityl-hexaethyleneglycol, 1-[(2-cyanoethyl)-(N,N-diisopropyl)]-phosphoramidite, M.W.: 784.93, 3-O-Dimethoxytrityl-propyl-1-[(2-cyanoethyl)-(N,Ndiisopropyl)]-Phosphoramidite, M.W.: 578.69, 12-0-Dimethoxytrityl-dodecyl-1-[(2-cyanoethyl)-(N,Ndiisopropyl)]-phosphoramidite, M.W.: 704.93 and 5′-O-Dimethoxytrityl-1′,2′-Dideoxyribose-3′-[(2-cyanoethyl)-(N,N-diisopropyl)]-phosphoramidite, M.W.: 620.73.

In this invention, the spacer phosphoramidites can be used to insert a mixed polarity 9 or 18 atom spacer arm in a nucleic acid. These compounds may be added in multiple additions when a longer spacer is required. The spacer phosphoramidites can also be added to substitute for bases within a nucleic acid sequence and to mimic an abasic site in an oligonucleotide.

Colored or Fluorescent Labeling.

Another preferred group of phosphoramidite reagents useful in this invention is any suitable colored or fluorescent labeling moiety. This includes any suitable 3′ or 5′-labelling reagent. Fluorescent derivatives are useful in tracking nucleic acids and/or the carrier in vivo or in vitro. Included are any fluorescein derivatives (i.e. 6-FAM, HEX and TET, derived from the 6-carboxy fluorescein isomer). Also included are any cyanine dye derivatives (i.e. Cy3 and Cy5 phosphoramidites) and phosphoramidite reagents with dabcyl or TAMRA labels.

Another preferred group of phosphoramidite reagents useful in this invention includes any suitable 3′ or 5′-Biotin phosphoramidite reagent for adding biotin to the nucleic acid to provide a specific coupling site with any suitable avidin or streptavidin. Biotin labeling phosphoramidites are capable of branching to allow multiple biotins to be introduced at the 3′- or 5′-terminus while biotin-dT can replace dT residues within the oligonucleotide sequence.

3′-Modified Nucleic Acids.

In the design and synthesis of antisense nucleic acids in this invention, there are preferred reagents for use in modifying the 3′-terminus of oligonucleotides. This may be achieved by modifying the 3′-terminus with a phosphate group, a phosphate ester, or using an inverted 3′-3′ linkage. Nucleic acids modified at the 3′-terminus resist 3′-exonuclease digestion and thereby provide a more effective agent in vivo.

A preferred group of phosphoramidite chemical reagents for 3′ phosphorylation includes 2-[2-(4,4′-Dimethoxytrityloxy)ethylsulfonyl]ethyl-(2-cyanoethyl)-(N,N-diisopropyl)-phosphoramidite, among others.

A preferred simpler process is to couple any suitable phosphoramidite reagent that is desired for modifying the 3′ end of a nucleic acid onto the support such as controlled pore glass (CPG). The coupled phosphoramidite is used as the starting compound for synthesizing the nucleic acid. Said coupling is designed for subsequent cleavage using suitable chemical methods to provide the desired 3′ modification.

A preferred method is described by H. Urata, et al, Tetrahedron Lett., 34, 4015-4018 (1993) for the preparation of oligonucleotides with a 3′-phosphoglyceryl terminus. The terminus is readily oxidized by sodium periodate to form a 3′-phosphoglycaldehyde. The aldehyde may be further oxidized to the corresponding carboxylic acid. Either the aldehyde or the carboxylate may be used for subsequent conjugation to amine-containing moieties.

Another preferred embodiment in synthesizing the compositions of this invention is to couple sense or antisense nucleic acids through the 3′-terminus. One preferred approach to 3′-modification is to prepare said nucleic acid with a ribonucleoside (RNA) terminus, (i.e. nucleic acid chimera) using an RNA support. Subsequent oxidation of the 2′,3′-diol cleaves the 2′-3′ bond and generates reactive aldehyde groups. The resulting 3′ aldehyde group is then available for specific coupling to suitable functional groups on the chloroquine substances and carrier substances of this invention. The nucleic acid methods and references disclosed within the following references are hereby incorporated into this invention.

1. B. A. Connolly, et al, Nucleic Acids Res., 1985, 13, 4485.

2. N. G. Dolinnaya, et al, Nucleic Acids Res., 1993, 21, 5403-5407.

3. G. B. Dreyer, et al, Proc. Natl. Acad. Sci. USA, 1985, 82, 968.

4. M. Durard, et al, Nucleic Acids Res., 1990, 18, 6353.

5. M. W. Kalnik, et al, Biochemistry, 1988, 27, 924-931.

6. M. Lemaitre, et al, Proc. Nat. Acad. Sci. USA, 1987, 84, 648.

7. M. Lemaitre, et al, Nucleosides & Nucleotides, 1987, 6, 311.

8. P. Li, et al., Nucleic Acids Res., 1987, 15, 5275.

9. M. Salunkhe, et al, J. Amer. Chem. Soc., 1992, 114, 8768-8772.

10. B. S. Sproat, et al, Nucleic Acids Res., 1987, 15, 4837.

11. M. Takeshita, et al, J. Biol. Chem., 1987, 262, 10171-10179.

12. R. Zuckerman, et al, Nucleic Acids Res., 1987, 15, 5305.

Synthesis Materials.

All chemicals were reagent grade and are available from Acros Organics/Fisher Scientific, Pittsburgh, Pa.; Alltech Assoc., Deerfield, Ill.; Amersham Pharmacia Biotech, Piscataway, N.J.; Calbiochem, San Diego, Calif.; Molecular Probes, Eugene, Oreg.; Promega Corp., Madison, Wis.; Sigma-Aldrich, St. Louis, Mo. 63178; TCI America, Portland Oreg.; or VWR International, West Chester, Pa. 19380. Deionized water is used where not stated otherwise.

Some reagents used and their abbreviations are; benzotriazol-1-yloxy-tris(dimethylamino)-phosphonium hexafluorophosphate (BOP), 1-Decene, n-butylamine, 2,2,2-trifluoroethanol, ethylenediamine tetraacetic acid (EDTA), 3-nitrophenol, fluorescein isothiocyanate (FITC), N-hydroxysuccinimide (NHS), ethanethiol, n-butylamine, 4-(dimethylamino)-pyridine (DMAP), dithiothreitol (DTT), 1,1,2-trichloroethane (TCE), sodium dodecyl sulfate (SDS) and 1,3-diisopropylcarbodiimide (DIC). Some solvents used are ethyl acetate (EtOAc), methanol (MeTOH), tetrahydrofuran (THF), N,N-dimethyl formamide (DMF), isopropanol and n-heptane. Phosphate-buffered saline (PBS) is 0.01 M sodium phosphate and 0.015 M sodium chloride, pH about 7.2 or adjusted with 0.1 M HCl, 0.1 M KOH (or NaOH) solution as needed.

Testing Procedures.

The chloroquine or chloroquine derivative concentration in the preparations was determined by fluorescence using 485 nm excitation wavelength and reading at 528 nm emission wavelength. The sample concentration was determined by using least squares (Linear regression) calculation of the slope and intercept from a standard curve of known concentrations.

The psoralen or trioxsalen concentration in the preparations was determined by fluorescence at 340 nm excitation wavelength and 528 nm emission wavelength. The sample concentration was determined by using least squares calculation of the slope and intercept from a standard curve as described previously.

Aldehyde concentration in the preparations was determined using the fluorescent indicator, 4′-Hydrazino-2-Stilbazole Dichloride (HSD) based on the method of S. Mizutani, et al, in Chem. Pharm. Bull. 17, 2340-2348 (1969). The sample concentration was determined by using least squares calculation as described previously.

Amine concentration was measured by the following colorimetric test. To 0.02 mL of amine sample in water was added 0.05 mL of borate buffer, pH 8.5. Then 0.05 mL of 0.075% 2,4,6-trinitrobenzene sulfonate (TNBS) was added and mixed. After 20 minutes at rt, the absorbance was read at 420 nm. The absorbance was compared to a glycine standard curve to calculate the sample amine concentration by least squares as described previously.

Carbohydrate concentration was measured by the following colorimetric test. To 0.02 mL of carbohydrate sample in water was added 0.01 mL of 1.5% naphthol in MetOH. Then 0.1 mL of concentrated sulfuric acid was added rapidly to mix. After 20 minutes at rt, the absorbance was read at 620 nm. The absorbance was compared to a dextran or CD standard curve to calculate the sample concentration by least squares as described previously.

Thiol concentration was measured by combining: 0.008 ml of sample and 0.1 ml of 0.0125% 2,2′-dithio-bis(5-nitropyridine) (DTNP) in 62.5% isopropanol, pH 6 to produce a color reaction. The absorbance was read at 405 nm and sample thiol concentration was calculated by linear regression using values from a cysteine standard curve.

### Coupling Methods Using Activated Esters

The following are methods for synthesizing the compositions of this invention. They are based on J. T. C. Wojtyk, et al, in Langmuir 18, 6081 (2002), for derivatizing a carboxylate group on any suitable carrier substance to provide an activated ester for coupling to a primary amine on a chloroquine substance, nucleic acid, intercalator or any suitable moiety.

Conversely, these methods can be used under suitable conditions for derivatizing a carboxylate group on any suitable chloroquine substance or active agent to provide an activated ester for coupling to a primary amine on a carrier substance as defined herein. For instance, with suitable modifications antibiotics can be coupled to a chloroquine substance using the methods disclosed by B. G. Knecht, et al, in Anal. Chem. 76, 646-654 (2004), and references therein.

If needed, a carrier substance with a hydroxyl or amino group such as protein, dextran, cyclodextrin or PEG is first carboxylated by reacting it with acetic (or succinic) anhydride in anhydrous solvent such as DMF. When suitable, any useful carbodiimide can be substituted for DIC.

A. Synthesis of 3-Nitrophenyl Activated Carrier Substance.

In a 100 mL round-bottom flask equipped with a magnetic stirrer and a nitrogen inlet is placed the carboxylated carrier substance such as pendant PEG with about 15 acid groups (1.00 g, 0.75 mmol acid) and 3-nitrophenol (0.14 g, 1.0 mmol). The mixture is dissolved in 10 mL of dry THF and cooled to 0° C. before a 10 mL THF solution of DIC (0.13 g, 1.0 mmol) and DMAP (0.012 g, 0.10 mmol) is added drop wise via a syringe over a 10 min period. The mixture is allowed to warm gradually to room temperature and stirred at this temperature for 18 h. The urea byproduct is filtered off and the filtrate is precipitated from isopropanol to recover the produce.

B. Synthesis of N-Succinimidyl Activated Carrier Substance.

In a 100 mL round-bottom flask equipped with a magnetic stirrer and a nitrogen inlet is placed the carboxylated carrier substance such as pendant PEG with about 15 acid groups (1.00 g, 0.75 mmol acid) and N-hydroxysuccinimide (0.12 g, 1.00 mmol). The mixture is dissolved in 5 mL of dry DMF and cooled to 0° C. before a 5 mL DMF solution of DIC (0.13 g, 1.0 mmol) and DMAP (0.012 g, 0.10 mmol) is added drop wise via a syringe over a 10 min period. The mixture is allowed to warm to room temperature and stirred for 18 h at this temperature. The urea byproduct is filtered off and the filtrate is precipitated from isopropanol to recover the produce.

C. Synthesis of S-Ethyl Activated Carrier Substance.

In a 100 mL round-bottom flask equipped with a magnetic stirrer and a nitrogen inlet is placed the carboxylated carrier substance such as pendant PEG with about 15 acid groups (1.00 g, 0.75 mmol acid) and ethanethiol (0.06 g, 1.00 mmol). The mixture is dissolved in 10 mL of dry THF and cooled to 0° C. before a 10 mL THF solution of DIC (0.13 g, 1.0 mmol) and DMAP (0.012 g, 0.10 mmol) is added drop wise via a syringe over a 10 min period. The mixture is stirred for 18 h at 0° C. The urea byproduct is filtered off and the filtrate is precipitated from isopropanol to recover the product.

D. Activated Ester Intercalator or Other Moiety.

With suitable modifications, the procedures used to add activated esters to the carboxylated carrier substances described previously, can also be used to add activated esters to carboxylated intercalators or other moieties. If needed, an intercalator or other moiety with a hydroxyl or amino group is first carboxylated by reacting it with acetic anhydride in anhydrous solvent such as DMF. These activated intercalators are then coupled to amino-derivatized carrier substances.

E. Coupling an Activated Intercalator to Amino-Containing Carrier Substances.

This procedure is used to conjugate amino-containing carrier substance (i.e. protein, amino-PEG or amino-dextran) with any suitable activated ester moiety including intercalators that have active ester (i.e. NHS) or isothiocyanate functional groups. At pH 9, conjugation occurs virtually exclusively at the amino group.

About 0.2 mmoles of amino-containing carrier substance (i.e., with about 0.1-0.2 mmoles of free primary amines) is dissolved in 1-2 mL of sterile distilled water. To this carrier solution is added 0.1-0.2 mL of 10× conjugation buffer (1M NaHCO₃/Na₂CO₃, pH 9).

A 10 mg/mL DMF solution is freshly prepared of the activated intercalator or moiety containing active ester or isothiocyanate. To the buffered carrier solution is added 0.2-0.4 mL of the DMF solution, mixed and allowed to stand at least 2 hours or overnight.

The reaction mixture is desalted on a column of Sephadex G-25 in water to remove the excess moiety. The product can be purified using reverse phase HPLC if necessary.

### Addition of Aldehyde Groups Using Glycidol

Carrier substances, chloroquine substances and any other suitable moiety that contains a hydroxyl, amino or sulfhydryl reactive group can be derivatized to provide an aldehyde functional group using this method. The substance is first derivatized by coupling glycidol (2,3 epoxy propanol, Sigma-Aldrich) to the reactive group. When coupled, the glycidol produces a “dihydroxy propyl” moiety (with two terminal, vicinal hydroxyl groups), coupled through an ether bond. Then the vicinal hydroxyl groups are oxidized with sodium periodate. The periodate oxidizes and cleaves the vicinal hydroxyls, leaving a terminal aldehyde group.

To an aqueous or nonaqueous solution of the substance to be derivatized is added glycidol at any desired molar ratio. For instance, to 100 mL of 1 mM NaOH in water (pH 8), containing about 8 gm of dissolved dextran 40 (TCI America), average mw 40,000 Daltons (40 kDa), was added 0.34 mL of glycidol (mw 74.02, 96%), mixed and put in the dark at rt for several days (CD159). The resulting dextran-glycidol preparation was concentrated by evaporation over boiling water to about 70 mL, giving a clear solution. The dextran-glycidol preparation was oxidized by adding about 0.94 gm of NaIO₄ in 10 mL of water, mixed and put in the dark at rt for about 1 hour. The resulting dextran-aldehyde was exhaustively dialyzed against water in suitable cellulose tubing (molecular weight cut-off of 12-14 kDa, Spectrum), for 3 days. The dextran-aldehyde dialysate was concentrated by evaporation to about 28 mL.

Alternatively, certain polysaccharides such as inulin or CD, can be oxidized without glycidol treatment to produce aldehydes. In any case, the aldehyde product, such as oxidized inulin or CD, can be collected by several precipitations with about 5 volumes of 100% isopropanol and cooling to −20° C. for several hours. The precipitate is collected by centrifugation and dissolved in water. Also, it can be further purified by Sephadex™ G50 size exclusion gel chromatography in water or water/MetOH (50%).

The product dry weight was 0.265 gm/mL, determined from drying a 0.10 mL aliquot to constant weight. Dextran (or inulin, CD) concentration is measured as carbohydrate as described herein. Aldehyde concentration is determined using HSD as described previously.

### Amination Methods

Carrier substances that do not normally contain amino groups can be suitably aminated to provide them by methods well known in the art as is disclosed for CD derivatives by A. R. Khan, et al, in Chem. Rev. 98, 1977-1996 (1998) and references therein which are hereby incorporated. For instance, carrier substances such as carbohydrates including inulins, dextrans and cyclodextrins, as well as PEG and other hydroxlated polymers with available hydroxyl groups are readily aminated through tosylation. The hydroxyl groups are first reacted with p-toluene sulfonyl chloride, in suitable anhydrous solvent. Then the tosylate on the reactive site is displaced by treatment with excess sodium azide. Finally, the azide is reduced to an amine with an appropriate hydrogenation method such as with hydrogen and a noble metal catalyst to provide an amino-containing carrier substance.

### Thiolation and Coupling Methods

On amino-containing carrier substances, chloroquine substances, intercalators and other moieties, the hydrazine or other amino groups can be thiolated to provide thiols for disulfide coupling such as between any suitable thiolated carrier substance and thiolated chloroquine substance, nucleic acid or intercalator. Suitable methods using SPDP or 2-iminothiolane are disclosed by E. J. Wawrzynczak, et al, in C. W. Vogel (ed.) Immunoconjugates; Antibody Conjugates in Radioimaging and Therapy of Cancer. NY; Oxford Univ. Press, pp 28-55, (1987).

For instance, primary amino groups on the carrier substance are thiolated in PBS, pH 7.5 by adding a 2× molar excess of SPDP in EtOH and letting it react for about 1 hour at rt. Excess SPDP is removed by size exclusion gel chromatography. Before coupling, the pyridine-2-thione is released by adding a molar excess of DTT to provide sulffiydryl groups.

Thiol-Disulfide Interchange. This is a method of this invention for coupling two thiolated moieties through their sulfhydryl groups to produce a disulfide linkage. For example, a thiolated carrier substance is first activated by reacting the sulfhydryls with a slight molar excess of 2,2′-dipyridyl disulfide (2DD), in suitable buffer (i.e. 0.1 M NaHCO₃, pH 8), for about 30 minutes. Depending on the type of carrier, the excess 2DD is removed by precipitation or gel exclusion chromatography. The 2DD-activated carrier substance is then combined with any suitable thiolated moiety in pH 8 buffer and reacted for 12-24 hours. The carrier substance with coupled moiety is collected by precipitation or chromatography as before.

### Intercalation Methods

These are general methods for coupling nucleic acids to carrier substances with coupled intercalators to produce intercalator-linked coupling of the nucleic acid. Preferably, intercalation is done in a small volume of water at a salt concentration of less than 20 mM, preferably 1-10 mM salt, pH 6-8, at room temperature. Based on previous determinations of intercalator concentration that is coupled to the carrier substance, an excess molar concentration of nucleic acid vs. intercalator is combined with the carrier.

For instance, for each micromole of coupled psoralen available on the carrier in 20 microliters of 0.002 M NaCO₃, 1.5-3 micromoles of oligodeoxynucleotide is added in about 20 microliters of water. Intercalation was allowed to proceed for about 1-2 hours at rt in the dark.

With photoreactive intercalators such as psoralen or trioxsalen, the intercalator-linkages can be converted into covalent linkages. For instance, the intercalated mixture is irradiated with 365 nm uv light (8 watt lamp about 6 cm above the solution surface) for about 15-45 minutes at rt. If desired, the optimal irradiation time for a given mixture can be determined empirically by comparing preparations using gel migration inhibition as described below.

The nucleic acid-loaded carrier is purified by collecting the leading fractions during size exclusion gel chromatography on a column of Sephadex G-50 in water or MetOH in water (i.e. 30-50% MetOH). Alternatively, the product can be purified by suitable precipitation methods or by using reverse phase HPLC if necessary.

### PREPARATION I

### Hydroxychloroquine Aldehyde Using Glycidol

(N42) To 4.33 grams (10 millimoles) of hydroxychloroquine (HQ) sulfate (Acros, 98%), dissolved in 25 mL of water was added about 3 mL of 0.1 N NaOH to adjust the pH to about 7.3. To this solution was added about 3.1 mL of glycidol (Sigma-Aldrich, 96%), for about a 4× molar excess of glycidol. The solution was mixed and put in the dark at room temperature (rt) for 48 hours or more to allow coupling of the glycidol to the hydroxyl groups.

The hydroxychloroquine-glycidol product was isolated by splitting the solution into 4 aliquots and diluting with about 6 volumes of isopropanol. The mixtures were placed in a −20° C. for several hours to allow precipitation, then centrifuged 30 minutes at about 2500 rpm. The pellets were dissolved in about 5 mL of water, pooled and precipitated as before, then dissolved in a final volume of 9.5 mL water.

The hydroxychloroquine-glycidol preparation was oxidized by adding 0.10 mL of about 0.16% NaIO₄ in water, mixed and left in the dark at rt for about 25 minutes to produce aldehyde groups. The resulting hydroxychloroquine-aldehyde (HQ-Ald) preparation was collected by repeated (2-3×), precipitations with isopropanol as described. HQ concentration was determined by fluorescence and aldehyde concentration was determined using HSD as described previously. Alternatively, the product is purified by Sephadex™ G50 size exclusion gel chromatography in water and concentrated by evaporation.

If desired, the coupled product is tested for purity using HPLC with an Xterra C₁₈ column (Waters Corp., Chicago Ill.) and a mobile phase of 15% acetonitrile in 25 mM ammonium formate, pH 6.5, flow rate 1 mL per minute. Purity is indicated by characteristic retention times when monitored by absorbance scanning at 300-360 nm and by refractive index.

Alternatively, other hydroxylated chloroquine analogs or amino-containing chloroquine substances can be substituted for the hydroxychloroquine. For instance, with suitable modifications, primaquine can be substituted for hydroxychloroquine in the above reaction to produce primaquine-aldehyde (PQ-Ald).

### PREPARATION II

### Hydroxychloroquine Amine Using Epoxypropylphthalimide

(N43) To 2.16 grams (5 millimoles) of hydroxychloroquine (HQ) sulfate (Acros, 98%), dissolved in 8 mL of water (pH 5), was added about 0.2 mL of 1 N NaOH to adjust the pH to about 6.5. To this solution was added about 25 mL of N-(2,3-epoxypropyl)phthalimide (EPP, Sigma-Aldrich, 98%), in 80% DMF/water, for about a 2× molar excess. The solution was mixed and put in the dark at room temperature (rt) for 48 hours or more to allow coupling of the EPP to the hydroxyl groups.

To remove the phthalate by hydrolysis, the pH was adjusted to about 9 with about 3 mL of 1 N NaOH. Then about 0.8 mL (2× molar excess) of hydrazine hydrate (64%, fw 50.06) was added, mixed and put in the dark at rt for 48 hours or more. The reaction mixture was then concentrated by evaporation. The hydroxychloroquine amine product was purified by Sephadex™ G15 size exclusion gel chromatography in 50% MetOH/water and concentrated by evaporation under N₂. HQ concentration was determined by fluorescence and amine concentration was determined using TNBS as described previously.

Hydroxychloroquine-Hydrazine. In another preferred hydroxychloroquine amine embodiment, hydroxychloroquine-aldehyde, disclosed herein, is coupled to excess hydrazine in water, to provide hydroxychloroquine-hydrazine with a biocleavable hydrazone linkage.

### PREPARATION III

### Quinacrine-Amine

In another preferred embodiment, quinacrine is sulfhydryl- or amino-derivatized, wherein any suitable diamino compound, including hydrazine are suitably coupled to quinacrine. For instance, to a solution of hydrazine (30 micromoles) in 4 mL of suitable solvent and/or aqueous buffer (i.e. 10 mM Hepes and 1 mM EDTA, pH 7.2), is added about 10 micromole of quinacrine mustard (Sigma-Aldrich) in 2 mL of solvent. The solution is mixed and left at rt in the dark for about 2 hours. The resulting product, quinacrine-coupled hydrazine, is purified by precipitation or by Sephadex™ gel exclusion chromatography. The quinacrine-hydrazine can then be coupled to any suitable carrier substance, or nucleic acid to produce a biocleavable hydrazone linkage.

Alternatively, a dimercapto compound such as dithiothreitol, can be coupled to quinactine mustard in place of a diamino compound, then coupled to any suitable carrier substance, or nucleic acid through thiol-disulfide interchange as disclosed herein, to produce a biocleavable disulfide linkage.

### PREPARATION IV

### Hydroxychloroquine-Coupled Trioxsalen

(N46) In this example, hydroxychloroquine aldehyde (HQ-Ald), is coupled to the amino group on trioxsalen. To a solution of trioxsalen (Calbiochem, 1.65 micromoles), in 0.5 mL of MetOH was added about 2.5 mL of HQ-Ald (about 2.2 micromoles), in about 30% solvent/water. The mixture was left at rt in the dark for over 48 hours, then reduced by adding about 0.15 mL of 20 mM NaBH₄ in water, mixed 2-3 hours, concentrated by evaporation and reconstituted in about 4 mL of 40% MetOH.

The HQ-coupled trioxsalen was purified by Sephadex™ G15 gel exclusion chromatography in 40% MetOH. The leading fractions contained HQ-coupled trioxsalen determined by the presence of both HQ fluorescence (excitation 485 nm; emission 528 nm), and trioxsalen fluorescence (excitation 340 nm; emission 680 nm) in the same elution peak ahead of either agent alone.

Alternatively, other chloroquine substances can be derivatized to provide an aldehyde group for coupling to trioxsalen. For instance, primaquine-aldehyde can be used in place of HQ-Ald to produce PQ-coupled trioxsalen.

Quinacrine-Coupled Trioxsalen. In another preferred embodiment, sulfhydryl- or amino-derivatized psoralens, including trioxsalen, are suitably coupled to quinacrine. For instance, to a solution of trioxsalen (2 micromoles) in 4 mL of suitable solvent and/or aqueous buffer (i.e. 50% DMF/10 mM Hepes and 1 mM EDTA, pH 7.2), is added about 1 micromole of quinacrine mustard (Sigma-Aldrich) in 1 mL of same solvent. The solution is mixed and left at rt in the dark for about 2 hours. The resulting product, quinacrine-coupled trioxsalen, is purified by precipitation or by Sephadex™ gel exclusion chromatography.

Intercalation. In any case, suitably one or more prepared chloroquine substance-coupled trioxsalens is easily coupled to any suitable nucleic acid through intercalation linkages between the trioxsalen and nucleic acid as disclosed previously. Through these methods, chloroquine substances are directly coupled to any suitable nucleic acid, including antisense ODN, or siRNA.

### PREPARATION V

### Biocleavable Primaquine-Coupled Combinative Agents

(N45) The chloroquine substance, primaquine (PQ), is derivatized with a bifunctional, amino cross linker 3, 3′-dithio-bis(propionate N-hydroxy succinimide ester), (DTSP, Sigma-Aldrich), which also contains a biocleavable, disulfide linkage. Alternatively, by coupling PQ to DTSP, a disulfide linkage is added which can be reduced with dithiothreitol to provide a sulfhydryl group on the PQ. Alternatively, the amino group on primaquine can be thiolated using 2-iminothiolane to provide thiolated chloroquine for disulfide coupling to any suitable thiolated carrier substance.

However, in this example, the DTSP will be used to cross link PQ to an amino-containing active agent to produce a new composition.

To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing biocleavable conjugates with the following combinative agents.

PQ-Dapsone. To about 0.26 gm (1 mmole) of 4,4-aminophenyl sulfone (dapsone, Sigma-Aldrich), in 10 mL of MetOH was added about 3 ml of PQ-DTSP solution (about 0.25 mmoles), mixed and left at rt in the dark for 24-48 hours. The resulting product, PQ-DTSP-dapsone conjugate is purified by Sephadex™ G15 gel exclusion chromatography and the leading fractions collected, pooled and concentrated by evaporation.

PQ-Pyrimethamine. To about 1 mmole of pyrimethamine (PRMA, Sigma-Aldrich), in suitable solvent, is added about 3 ml of PQ-DTSP solution (about 0.25 mmoles), mixed and left at rt in the dark for 24-48 hours. The resulting product, PQ-DTSP-pyrimethamine conjugate;is purified by precipitation or by Sephadex™ G15 gel exclusion chromatography and the leading fractions collected, pooled and concentrated by evaporation.

PQ-Penicillamine. To about 1 mmole of penicillamine (PNLA, Sigma-Aldrich), in suitable solvent, is added about 3 ml of PQ-DTSP solution (about 0.25 mmoles), mixed and left at rt in the dark for 24-48 hours. The resulting product, PQ-DTSP-penicillamine conjugate is purified by precipitation or by Sephadex™ G15 gel exclusion chromatography and the leading fractions collected, pooled and concentrated by evaporation.

Another preferred embodiment is biocleavable primaquine-coupled nucleic acid. Any suitable amino-derivatized nucleic acid with an available amino group (i.e. 3′-amino-ODN, or 3′-amino-RNA), is suitably combined with a solution of PQ-DTSP to produce PQ-DTSP-nucleic acid. The resulting PQ-nucleic acid then contains a biocleavable disulfide linkage between the PQ and the nucleic acid. Also, by incorporating multiple amino groups into said nucleic acid, several PQ moieties are coupled to said nucleic acid.

### PREPARATION VI

### Hydroxychloroquine-Coupled Nucleic Acid

In this example, HQ-aldehyde is directly coupled to a nucleic acid. Any suitable amino-derivatized nucleic acid with an available amino group (i.e. 3′- or 5′-amino-ODN, or 3′- or 5′-amino-RNA), is suitably combined with a solution of hydroxychloroquine-aldehyde (HQ-Ald) to produce HQ-nucleic acid. By derivatizing the nucleic acid with hydrazine groups and coupling to HQ-Ald, the resulting HQ-nucleic acid will then contain a biocleavable hydrazone linkage between the HQ and the nucleic acid. Also, by incorporating multiple amino groups (or hydrazines) into said nucleic acid, several HQ moieties are coupled to said nucleic acid.

(N46D) To 0.05 mL of an aqueous solution of 5′-amino-ODN (100 micrograms) was added about 0.16 mL of 12.5% HQ-Ald in water, then 0.01 mL of 0.02 M NaCO₃ to give about pH 7.5. The mixture was left at rt in the dark overnight. The Schiff's base couplings in the mixture were reduced by the addition of about 0.05 mL of 20 mM NaBH₄ solution.

The hydroxychloroquine-coupled nucleic acid product (HQ-nucleic acid) was purified by Superdex™ gel exclusion chromatography in water. The fractions were monitored for hydroxychloroquine fluorescence and.

The leading fractions contained HQ-coupled ODN determined by the presence of both HQ fluorescence (excitation 485 nm; emission 528 nm), and DNA absorbance (260 nm) in the same elution peak ahead of either DNA or HQ alone.

Quinacrine-Coupled Nucleic Acid. In another preferred embodiment, sulffiydryl- or amino-derivatized nucleic acids, including antisense ODN or siRNA, are suitably coupled to quinacrine. For instance, to a solution of 5′-amino-ODN (20 micromoles) in 4 mL of suitable solvent and/or aqueous buffer (i.e. 50% DMF/10 mM Hepes and 1 mM EDTA, pH 7.2), is added about 20 micromoles of quinacrine mustard (Sigma-Aldrich) in 4 mL of same solvent. The solution is mixed and left at rt in the dark for about 4 hours. The resulting product, quinacrine-coupled ODN, is purified by precipitation or by Sephadex™ gel exclusion chromatography.

### PREPARATION VII

### Primaquine Dextran Conjugates

In this example, dextran is derivatized using glycidol and oxidation to provide aldehyde groups for coupling to primaquine and other moieties.

A. Dextran-Aldehyde. To 1 mL of 15% dextran, average mw 40,000 Daltons (40 kDa) (Sigma-Aldrich), is added 0.1 mL of 1 M NaCO₃ to give a pH of about 12. To this solution is added about 0.012 mL of glycidol (40× molar), then put in the dark at rt for several days. The resulting dextran-glycidol preparation is oxidized by adding 0.05 gm of NaIO₄ and put in the dark at rt for about 2 hours. The resulting dextran-aldehyde is collected by precipitation with about 5 volumes of 100% isopropanol, cooling to −20° C. and centrifugation. The dextran-aldehyde precipitate is dissolved in water. Alternatively, it can be further purified by Sephadex™ G50 size exclusion gel chromatography in water. Aldehyde concentration is determined using HSD as described previously.

In another preferred embodiment, dextran or inulin, or other suitable polysaccharides can be suitably oxidized by this method without first coupling with glycidol. The resulting aldehyde containing polysaccharide can suitably be used in place of oxidized dextran in B, C or D, below.

B. Primaquine-Dextran. Primaquine is coupled to the dextran-aldehyde by adding about a two fold (2×) molar excess of primaquine to the dextran-aldehyde in water and put in the dark for several hours at rt. The resulting primaquine-dextran conjugate is purified by Sephadex™ G50 size exclusion gel chromatography in water.

C. Primaquine-Dextran-Poly Arginine Conjugate. Poly arginine (Sigma-Aldrich P4663, mw 10 kDa) is coupled to the remaining aldehydes on the primaquine-dextran-aldehyde by adding about a two or three fold molar excess of poly arginine (i.e. 1.6 micromoles in 0.32 mL water), to about 0.8 micromoles of primaquine-dextran-aldehyde in 0.5 mL water and about 0.040 mL of 0.02 M NaCO₃ for pH 8-9. The solution is mixed and put in the dark for several hours at rt. The resulting primaquine-dextran-poly arginine conjugate is purified by Sephadex™ G50 size exclusion gel chromatography in water or 50% MetOH in water.

Dextran concentration is measured as carbohydrate by a colorimetric test described previously. Poly arginine concentration is measured as amine by a colorimetric test for amines as described previously. Primaquine concentration is determined by fluorescence as described previously. Alternatively, inulins can be substituted for dextran to produce inulin-aldehyde.

D. Nucleic Acid Carrier. A nucleic acid carrier is prepared by coupling amino-derived nucleic acid to the dextran-aldehyde before step B or C, above. Alternatively, trioxsalen can be coupled to the dextran-aldehyde before step B or C, above. Then, a nucleic acid loaded carrier is prepared by the intercalation method described previously, combining the trioxsalen-dextran composition with suitable nucleic acid (i.e. ODN) at a molar ratio of 1:2 in water. The mixture is then uv irradiated before Sephadex™ G50 purification as disclosed previously.

### PREPARATION VIII

### Primaquine Cyclodextrin-Aldehyde Conjugates

In this example, a cyclodextrin (CD), containing aldehyde functional groups is first prepared. The CD-aldehyde can be from CD monomers, dimers, trimers or polymers previously coupled with glycidol (i.e. molar excess in water) as described herein.

A. CD-Aldehyde. To a glycidol coupled CD preparation in water (4% CD), sodium m-periodate (NaIO₄) was added directly while mixing at room temperature (rt.). The molar ratio of NaIO₄ to cyclodextrin was about 6:1, to oxidize the diols introduced with the glycidol and some of the secondary C2-C3 diols on the CD molecules. This produces multiple aldehydes per CD molecule. The reaction is continued at 30° C. in the dark for 6 hours to overnight. The resulting polyaldehyde CD preparation was purified by gel exclusion chromatography (G50 Sephadex™) in water, and concentrated by evaporation.

B. Primaquine-CD. Primaquine is coupled to the CD-aldehyde by adding about a two fold (2×) molar excess of primaquine to the CD-aldehyde in water and put in the dark for several hours at rt. The resulting primaquine-CD conjugate is purified by Sephadex™ G50 size exclusion gel chromatography in water or suitable MetOH/water solvent.

C. Primaquine-CD-Poly Arginine Conjugate. Poly arginine (Sigma-Aldrich P4663, mw 10 kDa) is coupled to the remaining aldehydes on the primaquine-CD-aldehyde by adding about a two or three fold molar excess of poly arginine to the primaquine-CD-aldehyde in water and put in the dark for several hours at rt. The resulting primaquine-CD-poly arginine conjugate is purified by Sephadex™ G50 size exclusion gel chromatography in water or 50% MetOH in water.

Alternatively, the CD aldehyde preparation in this example can be alpha, beta, or gamma cyclodextrin monomers, or dimers, trimers or polymers thereof, which have been suitably oxidized without pre-coupling to glycidol, to produce dialdehydes on the CD molecules. Also, other carbohydrates such as dextrans or inulins can be oxidized to provide aldehydes with or without pre-coupling to glycidol.

Cyclodextrin content is determined as carbohydrate as described previously. Poly arginine concentration is determined as amine as described previously. Psoralen concentration is determined fluorometrically as described previously.

D. Nucleic Acid Carrier. A nucleic acid carrier is prepared by coupling amino-derived nucleic acid to the CD-aldehyde before step B or C, above. Alternatively, trioxsalen can be coupled to the CD-aldehyde before step B or C, above. Then, a nucleic acid loaded carrier is prepared by the intercalation method described previously, combining the trioxsalen-CD composition with suitable nucleic acid (i.e. ODN) at a molar ratio of 1:2 in water. The mixture is then uv irradiated before Sephadex™ G50 purification as disclosed previously.

### PREPARATION IX

### Primaquine Lipid Conjugates and Micelles

In this example, primaquine is coupled to oleic acid by two different coupling methods. To each of two tubes (A and B), containing about 0.03 micromoles of primaquine (Sigma-Aldrich) is added about 1 mL of DMF, or other suitable solvent to dissolve.

A. To primaquine solution A, is added about 0.5 mL of 1:5 CH₂Cl₂: DMF containing about 0.045 micromoles of oleic anhydride (Sigma-Aldrich), vortexed and put in the dark at rt for about 24 hours to allow coupling of the oleic anhydride to the amino groups.

B. To primaquine solution B, is added about 0.05 mL of DMF containing about 0.045 micromoles of oleic acid N-hydroxysuccinimide ester (Sigma-Aldrich), vortexed and put in the dark at rt for about 24 hours to allow coupling of the oleic acid N-hydroxysuccinimide ester to the amino groups.

Both preparations A and B are quenched with about 0.005 mL of ethanolamine, vortexed and put in the dark at rt for about 24 hours. The resulting primaquine-oleic acid conjugates are purified by chromatography on C₁₈ columns using gradient elution of 10-100% acetonitrile in water. Primaquine concentration is determined by fluorescence using least squares calculation from a primaquine standard curve, as described herein. Preparations are stored at −20° C.

Nucleic acid loaded carrier is prepared by the intercalation method described previously, combining psoralen-lipid with suitable nucleic acid (i.e. ODN) at a molar ratio of 1:2 in water. The mixture is then uv irradiated before Sephadex™ G50 purification as disclosed previously. This preparation is incorporated into any suitable micelle or liposome formulation which can include other amphiphilic molecules as disclosed herein to provide the micelle or liposome carrier composition of this invention.

Quinacrine-Coupled Lipid. In another preferred embodiment, sulfhydryl- or amino-derivatized lipids, including stearylamines are suitably coupled to quinacrine. For instance, to a solution of stearylamine (20 micromoles) in 4 mL of suitable solvent and/or aqueous buffer (i.e. 50% DMF/10 mM Hepes and 1 mM EDTA, pH 7.2), is added about 20 micromoles of quinacrine mustard (Sigma-Aldrich) in 4 mL of same solvent. The solution is mixed and left at rt in the dark for about 4 hours. The resulting product, quinacrine-coupled stearylamine, is purified by precipitation or by Sephadex™ gel exclusion chromatography.

### PREPARATION X

### Biocleavable Primaquine-Gamma Globulin Conjugate

In this example (N45B), primaquine is coupled to gamma globulin protein to provide a biocleavable primaquine protein carrier. Nucleic acid can then be coupled to the gamma globulin.

To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing a biocleavable conjugate with the gamma globulin.

To about 0.2 mg of human gamma globulin (Sigma-Aldrich) in about 0.8 mL of 0.002 M NaCO₃, pH 8, is added about 3 ml of PQ-DTSP solution (about 0.25 mmoles), mixed and left at rt in the dark for 24-48 hours. The resulting product, PQ-DTSP-gamma globulin conjugate is purified by Sephadex™ G15 gel exclusion chromatography and the leading fractions collected, pooled and concentrated. Primaquine concentration is determined by fluorescence vs. protein concentration determined by amino assay as described previously.

Alternatively, hydroxychloroquine-aldehyde or primaquine-aldehyde can be coupled to the antibody through available amino groups on the protein.

Oxidized Gamma Globulin. In another preferred embodiment, the carbohydrate moiety of the gamma globulin, is suitably oxidized to provide aldehydes using either NaIO₄ (A. Murayama, et al, Immunochem. 15, 532, 1978), or a suitable oxidizing enzyme such as glucose oxidase. Then, primaquine or suitably, hydroxychloroquine-hydrazine is coupled to the aldehydes on the protein to provide a biocleavable hydrazone linkage.

For instance, to about 3 mg of gamma globulin in 3 mL of PBS, pH 6.2, is added about a 50× molar excess of NaIO₄ and mixed. After reacting for about 1 hour at 4° C., the reaction is quenched with about 30× molar excess of ethylene glycol. The oxidized globulin is collected by ultra filtration (50 kDa MWCO) and reconstituted in PBS.

To the oxidized globulin is added a 20× molar excess of primaquine is suitable solvent and allowed to couple for 2-3 hours in the dark at rt. The resulting PQ-Globulin is purified by Sephadex™ gel chromatography.

Alternatively, this procedure, with suitable modifications, can be used to produce oxidized antibody. Also, other glycoproteins can be substituted for the gamma globulin.

### PREPARATION XI

### Quinacrine-Coupled Antibody

In a preferred embodiment, sulffiydryl- or amino-derivatized antibodies, are suitably coupled to quinacrine. For instance, to a solution of antibody (100 micromoles) in 4 mL of suitable aqueous buffer (i.e. 10 mM Hepes and 1 mM EDTA, pH 7.2), is added about 10 micromole of quinacrine mustard (Sigma-Aldrich) in 1 mL of same solvent. The solution is mixed and left at rt in the dark for about 2 hours. The resulting product, quinacrine-coupled antibody, is purified by ammonium sulfate precipitation and/or by Sephadex™ gel exclusion chromatography.

### PREPARATION XII

### Biocleavable Primaquine-Peptide Conjugate

In this example (N45), primaquine is coupled to a polylysine peptide to provide a primaquine-peptide carrier. To a solution of about 0.25 gm (1 mmole) of primaquine in 12.5 mL of about 60% DMF and 12% DMSO in water, was added about 0.35 gm (0.9 mmoles) of DTSP in 6 mL of about 16% CH₂Cl₂ in DMF. The solution of PQ-DTSP was mixed and put in the dark at rt for about 3 hours before preparing a biocleavable conjugate with the gamma globulin.

To a solution of polylysine (1 millimole) in about 10 mL of suitable solvent and/or aqueous buffer (0.002 M NaCO₃, pH 8), is added about 3 ml of PQ-DTSP solution (about 0.25 mmoles), mixed and left at rt in the dark for 24-48 hours. The resulting product, PQ-DTSP-peptide conjugate is purified by Sephadex™ G15 gel exclusion chromatography and the leading fractions collected, pooled and concentrated. Primaquine concentration is determined by fluorescence vs. peptide concentration determined by amino assay as described previously.

Alternatively, hydroxychloroquine-aldehyde or primaquine-aldehyde can be coupled to the peptide through available amino groups. Also, any suitable peptide, such as those containing lysine or arginine, with one or more available amino groups, can be substituted for the peptide in this example. Preferably, nucleic acid (i.e. DTSP-coupled ODN) can also be coupled to the peptide through biocleavable linkages.

Quinacrine-Coupled Peptide. In another preferred embodiment, sulfhydryl- or amino-containing peptides such as those containing lysine, arginine or cysteine (or are suitably derivatized) are suitably coupled to quinacrine. For instance, to a solution of polylysine or polyarginine (4 micromoles) in 4 mL of suitable solvent and/or aqueous buffer (i.e. 50% DMF/10 mM Hepes and 1 mM EDTA, pH 7.2), is added about 1 micromole of quinacrine mustard (Sigma-Aldrich) in 1 mL of same solvent. The solution is mixed and left at rt in the dark for about 2 hours. The resulting product, quinacrine-coupled peptide, is purified by precipitation or by Sephadex™ gel exclusion chromatography.

### PREPARATION XIII

### Primaquine-PEG Conjugate

In this example, primaquine is coupled to a diepoxy PEG to provide a primaquine-PEG (PQ-PEG) conjugate. The conjugate is then thiolated to provide sulfhydryl groups for coupling other moieties.

A. Primaquine-PEG. To about 0.03 micromoles of primaquine (Sigma-Aldrich) in about 10 mL of DMF is added about 700 micrograms (0.03 micromoles) of polyethylene glycol diglycidyl ether, “PEG-DE”, mw about 23,250 (Sigma-Aldrich #47,569-6). The solution is mixed and put in the dark at rt for 3-4 days.

Remaining epoxy groups are quenched by adding 30 micrograms (0.12 micromoles) of sodium thiosulfate in 0.010 mL water, mixed and kept at rt in the dark for 2 days. To this solution is added about 0.23 milligrams of dithiothreitol (DTI) in about 1 mL of water, mixed and kept at rt in the dark for about 3 hours to reduce coupled sodium thiosulfate to sulfhydryl groups on the PQ-PEG conjugate.

B. Purification. The preparation is fractionated by size exclusion gel chromatography on a Sephadex™ G25 column in suitable solvent (i.e. 10% MetOH/water) as the mobile phase. Fractions are collected and monitored for primaquine fluorescence as described previously. The leading fractions that contain PEG with primaquine fluorescence indicate that PQ is coupled to the PEG. The PQ-PEG fractions are pooled and concentrated by evaporation in the dark, under flowing nitrogen.

Alternatively, the PEG-DE is first coupled to hydrazine through the epoxy groups to produce PEG-hydrazine. Then hydroxychloroquine-aldehyde or primaquine-aldehyde is coupled to the hydrazine on the PEG to provide acid labile linkages as described previously. Alternatively, any suitable diamino compound can be used in place of hydrazine, and/or primaquine or hydroxychloroquine-amine can be coupled to the PEG-hydrazine through suitable cross linkers.

Alternatively, the PEG-DE is first coupled to sodium thiosulfate through the epoxy groups, then reduced with DTT to produce sulfhydryl-PEG. Then sulfhydryl derivatized (thiolated) primaquine or sulfhydryl derivatized (thiolated) hydroxychloroquine is coupled to the sulffiydryl groups on the PEG to provide biocleavable disulfide linkages as described previously.

### PREPARATION XIV

### Pendant PEG-Hydrazine For Biocleavable Linkages

In this example, pendant polyethylene glycol (SunBio USA, mw 20 KDa) with approximately 15 propionic acid side chains (PaPEG) is coupled to hydrazine through available carbonyl groups on the PEG. This provides side chains with terminal hydrazine moieties. The hydrazine groups can then be coupled to moieties containing aldehyde groups to provide biocleavable, acid-labile hydrazone linkages.

A. PaPEG-Hydrazine. Into about 20 ml of water, about 5 gm of pendant PEG was dissolved, the pH was about 5. Based on the manufacturer's value of 15 moles of propionic acid per mole of PaPEG, there was about 0.375 mmoles of carboxylic acid present. In a separate container, 1.8 ml of hydrazine hydrate (64%, fw 50.06) was neutralized to pH 7 with about 6.25 ml of 5N HCl, to give a final concentration of about 0.225 ml hydrazine per ml of solution.

A thirty-fold molar excess (30×) of hydrazine (4 ml of hydrazine solution) was added to the PaPEG solution and mixed with a magnetic stirrer. After about 2 minutes, a twenty-fold molar excess (20×=1.45 gm) of N-(3-Dimethylaminopropyl)-N′-Ethylcarbodiimide (EDC, fw 191.7), was added to the solution of PaPEG and mixed thoroughly. The pH was about 6. The solution was allowed to react overnight at room temperature (rt).

B. Purification. The reaction mixture was fractionated on a Sephadex™ G25 column equilibrated and eluted with 0.005 M HCl in water. The fractions are analyzed for refractive index. They are also analyzed for primary amine using a colorimetric test described previously. The leading fractions with corresponding high refractive index and amine content are pooled and concentrated by evaporation under nitrogen gas. The resulting product (PaPEG-Hzn), is PaPEG with hydrazine functional groups covalently coupled to the propionic acid moieties.

The PaPEG-Hzn can now have any suitable moiety with a terminal aldehyde group coupled to the available hydrazine groups. This will provide an acid labile hydrazone linkage described herein. Alternatively, any suitable diamino compound can be used in place of hydrazine.

Alternatively, any suitable chloroquine substance, intercalator, or other moiety with a terminal active ester can be coupled to the amine as described herein. Also, using suitable bifunctional amino coupling agents described herein, any suitable amino derivatized nucleic acids or intercalator can be covalently coupled to the hydrazine (or amino) moieties.

Alternatively, the hydrazine (or amino) groups can be thiolated using SPDP or 2-iminothiolane as described herein to provide thiols for disulfide coupling to any suitable thiolated nucleic acid.

Also, using coupling agents described herein, the terminal hydrazine groups can be coupled to a diamino, Fmoc half-protected biocleavable peptide containing any suitable biocleavable sequence such as GFLG, Phe-Leu, Leu-Phe or Phe-Phe, among others. The Fmoc groups are then removed to provide unprotected amino groups for subsequent coupling to an intercalator.

Alternatively, said biocleavable peptide can include a sulfhydryl group at one end for subsequent coupling to a thiolated nucleic acid (i.e. disulfide coupling), or amino-derivatized nucleic acid using a bifunctional cross linking agent.

Alternatively, the hydroxyl end groups on the PEG backbone can be suitably derivatized and coupled to suitable targeting molecules, transduction vectors, or grafted polymers using other coupling groups such as succinimide, N-succinimidyl, bromoacetyl, maleimide, N-maleimidyl, oxirane, p-nitrophenyl ester, or imidoester. Also, aldehydes that are coupled to hydrazine to give amino-aldehyde (Schiff's base) bonds can be reduced with NaBH₄ to stabilize them.

### PREPARATION XV

### Chloroquine Substance-Coupled PaPEG-Nucleic Acid Carrier

In this example, hydroxychloroquine-aldehyde (HQ-Ald) and ODN-aldehyde are coupled to the available hydrazine groups on pendant-PEG-Hzn (PaPEG-Hzn) to provide a chloroquine substance-coupled nucleic acid carrier with acid labile hydrazone linkages.

A. Preparation of ODN-Aldehyde.

Any suitable amino- or sulffiydryl-derivatized nucleic acid can be used in this example. For instance, any phosphorothioate antisense ODN (i.e. anti-bcl2, G3139), such as with a 5′ extension of 1 or more phosphodiester or phosphorothioate thymidine bases with a terminal amino group. An exemplary sequence and composition are as follows;

5′-Amino-(T)n-TCT CCC AGC GTG CGC CAT-3′;

Wherein N is an integer between 1 and 20.

A solid phase extraction (SPE) column containing 500 mg of C18 solid phase is preconditioned with 3 mL of MetOH and 2 mL of water. Then 0.02 micromoles of amino-derivatized oligodeoxynucleotide (ODN), in 0.1 mL of water is applied and allowed to soak into the column bed, followed with 0.2 mL more water. Then about 0.1 mL of 3% glutaraldehyde is applied, followed with 0.1 mL water. The column bottom is plugged and allowed to stand for 30 minutes. The column is then washed with 1.0 mL of water, followed with 1 mL of 5% MetOH and 1.5 mL of 10% MetOH.

The resulting ODN-aldehyde (ODN-Ald) is then eluted with about 3 mL of 100% MetOH and concentrated by evaporation in the dark, under flowing nitrogen to about 1 mL. The ODN-Ald is tested for purity using HPLC with an Xterra C18 column as described previously. Aldehyde concentration is determined colorimetrically using HSD and ODN is monitored by absorbance at 260 nm.

Alternatively, any amino- or sulfhydryl-derivatized RNA can be substituted for the ODN in this example.

B. Coupling ODN-Ald to PaPEG-Hzn.

The ODN-Ald is combined with a slight molar excess of PaPEG-Hzn (i.e. 1.5×), in water, based on amino content vs. ODN-Ald concentration. The reaction mixture is allowed to proceed for about 4 hours in the dark.

The PEG-ODN conjugate is purified by precipitation with 100% isopropanol at −20° C. and centrifugation. The pellet is dissolved and fractionated on a Sephadex™ G25 “mini” column (bed=0.7×4.8 cm) with 50% MetOH in water.

C. Coupling HQ-Ald to PEG-ODN.

In this step the remaining hydrazine groups on the PEG-ODN from the previous step are coupled with hydroxychloroquine-aldehyde (HQ-Ald), prepared previously as described herein.

The HQ-Ald in molar excess (i.e. 3×) is combined with PEG-ODN in water, based on HQ-Ald concentration vs. amino content of the PEG-ODN. The reaction mixture is allowed to proceed for about 4 hours in the dark.

The resulting HQ-PEG-ODN conjugate is purified by precipitation with 100% isopropanol at −20° C. and centrifugation. The pellet is dissolved in 2 mL of water and fractionated on a Sephadex™ G25 “mini” column (bed=0.7×4.8 cm) with 50% MetOH in water (or 2 mM NH₄ formate, pH 7). The fractions are monitored for hydroxychloroquine fluorescence as described herein and ODN is monitored by absorbance at 260 nm. The leading fractions with the highest fluorescence are pooled and concentrated by precipitation as described.

In another embodiment, other moieties such as transduction vectors, amphiphilic molecules and grafted polymers are coupled to said carrier in addition to the chloroquine substance and ODN.

In another preferred embodiment, said hydrazine-linked or diamino-linked PaPEG is thiolated before coupling through disulfide linkages to a thiolated chloroquine substance, nucleic acid, intercalator, targeting molecule, transduction vector, or other moiety.

The amino groups on the PaPEG are thiolated in PBS, pH 7.5 by adding a 2× molar excess of SPDP in EtOH and letting it react for about 1 hour at rt. Excess SPDP is removed by size exclusion gel chromatography. Before coupling, the pyridine-2-thione is released by adding a molar excess of DTT to provide sulfhydryl groups. Alternatively, other suitable amino-containing carrier substances can be substituted for the PaPEG.

### PREPARATION XVI

### Chloroquine Substance-Coupled PaPEG-RNA Carrier

RNA-Coupled Pendant Carboxylated PEG. In this example, pendant PEG (PaPEG), with pendant carboxylic acid groups (i.e. propionic acid), described previously, is coupled to RNA in addition to a chloroquine substance. Any suitable RNA can be used in this example. For instance, suitably derivatized RNA (i.e. siRNA targeted to Bcl-2), such as a double-stranded 21 mer RNA. Wherein the “Sense” strand (S), has a two base (2dT) overhang and a terminal amino functional group at the 3′ end (amino-RNA) and the “Antisense” strand (AS), only has a two base (2dT) overhang at the 3′ end. The siRNA targeted to Bcl-2 contains the following sequences;

| | |
|---|---|
| Sense strand | 5′-UGUGGAUGACUGAGUACCUGAdTdT-Amino-3′ |
| (S) | |
| Antisense | 5′-UCAGGUACUCAGUCAUCCACAdTdT-3′ |
| strand (AS) | |

First, the S strand RNA is coupled through the amine group at the 3′ end to the carbonyl group on the propionic acid side chain of PaPEG.

To 10 ml of 0.05 M phosphate buffer, pH 7.5, containing about 2 gm of pendant PEG is added an equimolar molar or less of amino-RNA and mixed. After about 5 minutes, a molar excess (20×) of N-(3-dimethyl aminopropyl)-N′-ethylcarbodiimide (EDC), is added, mixed and left to react 1-2 hours at rt. This reaction mixture can be taken to the next step.

Optionally, the resulting product, RNA-PaPEG, is purified by Sephadex™ G25 gel exclusion chromatography in water. The concentration of S strand RNA is determined by measuring absorbance at 260 nm.

Coupling Primaquine to PEG-RNA.

In this step the remaining carboxyl groups on the PEG-RNA from the previous step are coupled with primaquine (PQ). The PQ in molar excess (i.e. 3×) is added to the PEG-RNA and EDC reaction mixture, based on PQ concentration vs. amino content of the PEG-RNA. The reaction mixture is allowed to proceed for about 4 hours in the dark.

The resulting PQ-PEG-RNA conjugate is purified by precipitation with 100% isopropanol at −20° C. and centrifugation. The pellet is dissolved in 2 mL of water and fractionated on a Sephadex™ G25 “mini” column (bed=0.7×4.8 cm) with 50% MetOH in water (or 2 mM NH₄ formate, pH 7). The fractions are monitored for primaquine fluorescence as described herein and RNA is monitored by absorbance at 260 nm. The leading fractions with the highest fluorescence are pooled and concentrated by precipitation as described.

The double stranded siRNA is produced on the PaPEG carrier by hybridizing the AS RNA strand to the previously coupled S RNA strand using suitable hybridization conditions.

Biocleavable RNA-Coupled Pendant PEG Hydrazine. In this example, hydrazine-linked or diamino-linked PaPEG described previously is coupled to any suitable RNA in addition to a chloroquine substance. The S strand RNA is coupled through a hydrazone linkage to produce an acid labile linkage with the RNA. The double stranded siRNA will be generated by hybridizing the AS strand to the conjugated S strand.

RNA Aldehyde. A solid phase extraction (SPE) column containing 500 mg of C18 solid phase is preconditioned with 3 mL of MetOH and 2 mL of water. Then about 0.02 micromoles of amino-derivatized RNA, in 0.1 mL of water is applied and allowed to soak into the column bed, followed with 0.2 mL more water. Then about 0.1 mL of 3% glutaraldehyde is applied, followed with 0.1 mL water. The column bottom is plugged and allowed to stand for 30 minutes. The column is then washed with 1.0 mL of water, followed with 1 mL of 5% MetOH and 1.5 mL of 10% MetOH.

The resulting RNA-aldehyde (RNA-Ald) is then eluted with about 3 mL of 100% MetOH and concentrated by evaporation in the dark, under flowing nitrogen to about 1 mL. The RNA-Ald is tested for purity using HPLC with an Xterra C18 column as described previously. Aldehyde concentration is determined colorimetrically using HSD and RNA is monitored by absorbance at 260 nm.

Coupling RNA-Ald to PaPEG-Hzn.

The RNA-Ald is combined with a slight molar excess of PaPEG-Hzn (i.e. 1.5×), in water, based on amino content vs. RNA-Ald concentration. The reaction mixture is allowed to proceed for about 4 hours in the dark.

The PEG-RNA conjugate is purified by precipitation with 100% isopropanol at −20° C. and centrifugation. The pellet is dissolved and fractionated on a Sephadex™ G25 “mini” column (bed=0.7×4.8 cm) with 50% MetOH in water.

Coupling HQ-Ald to PEG-RNA.

In this step the remaining hydrazine groups on the PEG-RNA from the previous step are coupled with hydroxychloroquine-aldehyde (HQ-Ald), prepared previously as described herein.

The HQ-Ald in molar excess (i.e. 3×) is combined with PEG-RNA in water, based on HQ-Ald concentration vs. amino content of the PEG-RNA. The reaction mixture is allowed to proceed for about 4 hours in the dark.

The resulting HQ-PEG-RNA conjugate is purified by precipitation with 100% isopropanol at −20° C. and centrifugation. The pellet is dissolved in 2 mL of water and fractionated on a Sephadex™ G25 “mini” column (bed=0.7×4.8 cm) with 50% MetOH in water (or 2 mM NH₄ formate, pH 7). The fractions are monitored for hydroxychloroquine fluorescence as described herein and RNA is monitored by absorbance at 260 nm. The leading fractions with the highest fluorescence are pooled and concentrated by precipitation as described.

The double stranded siRNA is produced on the PaPEG carrier by hybridizing the AS RNA strand to the previously coupled S RNA strand using suitable hybridization conditions.

In another embodiment, other moieties such as transduction vectors, amphiphilic molecules and grafted polymers are coupled to said carrier in addition to the chloroquine substance and RNA.

In another embodiment, said hydrazine-linked or diamino-linked PaPEG is thiolated before coupling through disulfide linkages to a thiolated chloroquine substance, nucleic acid, intercalator, targeting molecule, transduction vector, or other moiety.

The amino groups on the PaPEG are thiolated in PBS, pH 7.5 by adding a 2× molar excess of SPDP in EtOH and letting it react for about 1 hour at rt. Excess SPDP is removed by size exclusion gel chromatography. Before coupling, the pyridine-2-thione is released by adding a molar excess of DTT to provide sulfhydryl groups. Alternatively, other suitable amino-containing carrier substances can be substituted for the PaPEG.

### PREPARATION XVII

### Maleimido or Iodo Carrier Substances Coupled to a Thiolated Moiety

In this example, an amino-containing carrier substance is derivatized to contain a maleimide or an iodo reactive group. Then a chloroquine substance, nucleic acid, intercalator, targeting molecule, transduction vector or other moiety is suitably thiolated as described herein before coupling it to the derivatized carrier substance. There are well known methods for derivatizing the primary amine on the carrier substance (i.e. protein, PEG) to provide a maleimido group. For instance, a bifunctional (succinimidyl-maleimido) cross linker described herein, such as MBS or SMPB is coupled to the primary amine to provide free maleimide groups. Upon reaction with a thiolated moiety, a stable thioether bond is formed.

Alternatively, iodo-carrier substances such as iodo-polyethylene glycol (Iodo-PEG) carriers can be prepared for coupling to a sulfhydryl group on a chloroquine substance, nucleic acid, intercalator, targeting molecule, transduction vector or other moiety. For instance, NHS esters of iodoacids can be coupled to the amino-containing carrier substances. Suitable iodoacids for use in this invention are iodopropionic acid, iodobutyric acid, iodohexanoic acid, iodohippuric acid, 3-iodotyrosine, among others. Before coupling to the amino-carrier substance, the appropriate Iodo-NHS ester is prepared by known methods. For instance, equimolar amounts of iodopropionic acid and N-hydroxysuccinimide are mixed, with suitable carbodiimide, in anhydrous dioxane at RT for 1-2 Hrs, the precipitate removed by filtration, and the NHS iodopropionic acid ester is collected in the filtrate. The NHS iodopropionic acid ester is then coupled to the amino-carrier substance.

### PREPARATION XVIII

### Amphiphilic Cyclodextrin

In this example, a mixture of amphiphilic cyclodextrin dimers, trimers and polymers with alkyl carbon chains attached is prepared for use as carrier substances. The cyclodextrins are cross-linked through hydroxyl groups using 1,4 butanediol diglycidyl ether (BDDE). Excess BDE molecules coupled at one end to the CD provide terminal oxirane groups that are subsequently thiolated by reaction with thiosulfate and reduction. Alkyl carbon chains are coupled to the CD derivatives using a “long chain epoxy” that couples to other available hydroxyl groups (CD88).

A. Cross-linking with BDDE. Into 125 ml of hot water (70-80° C.) adjusted to pH 4.5-5 with 0.05 ml 6 N HCl, is dissolved 2.84 gm of beta cyclodextrin (0.0025 moles). To this solution 4.1 ml of BDDE (about 0.0125 moles) is added with mixing and continued heating for about 2 hours.

B. Coupling with a Long Chain Epoxy. The mixture is adjusted to pH>10 with 1 M KOH and 1.28 gm (about 0.005 moles) of dodecyl/tetradecyl glycidyl ether (DTGE) is added and mixed vigorously. The solution is periodically mixed for about 1.5 hours, heated for about 3 hours and then left at room temperature (rt) overnight. The resulting solution is light yellow and turbid.

C. Thiolation with Na Thiosulfate. To the reheated mixture, 6 gm (about 0.025 moles) of sodium thiosulfate is added and mixed. After about 1 hour, the pH is adjusted to 7 with KOH and the solution was heated for about 3.5 hours more. Excess DTGE was removed by chilling to solidify the DTGE and the solution was decanted. The mixture was dialyzed against a continuous flow of distilled water in 500 molecular weight cutoff (mwco) tubing (Spectra/Por CE) for about 40 hours. The solution was concentrated by evaporation to 8 ml to give a clear, light yellow solution.

To the mixture, 8 ml of water and 0.96 gm (about 0.0062 moles) of dithiothreitol (DTT) was added, mixed and left overnight. The turbid solution was then dialyzed against a continuous flow of distilled water in 500 mwco tubing (Spectra/Por CE) for about 40 hours. The solution was concentrated by evaporation to 3.7 ml to give a clear, yellow solution. Total yield based on dry weight was 2.276 gm.

D. Column Chromatography. The mixture was fractionated on a Sephadex™ G15 column (2.5×47 cm) in water. The fractions are tested for relative carbohydrate and thiol concentration as described previously. Fractions with corresponding peak concentrations of carbohydrate and thiol are pooled and concentrated by evaporation. The final volume was 2.2 ml and the total yield based on dry weight was 1.144 gm. The resulting amphiphilic CD polymer was highly water soluble and amorphous (glassy) when dried.

E. Coupling With Thiolated Moieties. The amino groups on moieties such as amino-derivatized chloroquine substances (i.e. primaquine), or trioxsalen and other amino-moieties can be thiolated using SPDP or 2-iminothiolane as described previously. The thiolated moieties are then coupled to the carrier substance through disulfide linkages using thiol-disulfide interchange as described previously. Also, other thiolated moieties such as targeting molecules, transduction vectors and grafted polymers can be coupled through disulfide linkages.

Alternatively, to produce other suitable hydrophobic CD derivatives, other alkyl chains can be introduced by substituting suitable alkyl epoxy compounds for the one used in this example. For instance 1,2-epoxy derivatives of any suitable alkane such as propane, butane, pentane, hexane, octane, decane and dodecane can be substituted. Other useful epoxies such as glycidyl isopropyl ether, glycidyl methacrylate and glycidyl tosylate can be substituted. Also certain aromatic epoxies or heterocyclic epoxies can be substituted such as benzyl glycidyl ether, (2,3-epoxypropyl)benzene, 1,2-epoxy-3-phenoxypropane, exo-2,3-epoxynorborane, among others.

Alternatively, the CD polymer can be suitably derivatized with other coupling groups such as succinimide, N-succinimidyl, bromoacetyl, maleimide, N-maleimidyl, oxirane, p-nitrophenyl ester, or imidoester. Also, the CD polymer can be coupled to a polypeptide containing any suitable biocleavable sequence such as Phe-Leu, Leu-Phe or Phe-Phe, among others. Also, the CD polymer can be suitably derivatized to provide a CD-block with an N carboxyanhydride for subsequent copolymerization into PEO-block copolymers.

Combinations for this invention can include the covalent coupling of an alpha CD with a beta CD, an alpha CD with a gamma CD, a beta CD with a gamma CD and polymers with various ratios of alpha, beta and gamma cyclodextrins.

F. Intercalation. Nucleic acid loaded carrier is prepared by the intercalation method described previously, combining trioxsalen-CD with suitable nucleic acid (i.e. ODN) at a molar ratio of 1:2 in water. The mixture is then uv irradiated before Sephadex™ G50 purification as disclosed previously. This preparation is incorporated into any suitable micelle or liposome formulation which can include other amphiphilic molecules as disclosed herein to provide the micelle or liposome carrier composition of this invention.

### PREPARATION XIX

### Nucleic Acid Carriers from Hydroxylated Polymers

These are methods for synthesizing nucleic acid carrier compositions to provide for coupling to any suitable intercalator, targeting molecule, transduction vector, or other moiety with a suitable functional group. The targeting molecule can be a suitable protein, including antibodies, lectins, avidins and streptavidin, or ligands.

A. Preparation of NHS-Carrier Substances. A carrier substance with available hydroxyl groups such as carbohydrates (i.e. CD, or inulin), PEG and other grafted polymers described herein, is derivatized to provide an NHS ester. In a suitable anhydrous solvent such as DMF, the carrier substance is coupled to acetic anhydride and purified as described herein, to provide carboxyl groups. Then, the carboxylic acid group is reacted with N-hydroxysuccinimide and an aromatic carbodiimide such as N,N-dicyclohexyl carbodiimide, at approximately equimolar ratios and reacted at rt for 1-3 Hrs. The product, N-hydroxysuccinimide carrier (i.e. NHS-PEG), is separated in the filtrate from precipitated dicyclohexylurea, collected by evaporation and purified by chromatography.

Under appropriate conditions, NHS-carrier substances can be prepared by coupling NHS esters directly to an amino derivatized carrier substance. Preferably, the NHS ester is a bifunctional NHS coupling agent with a suitable spacer. Suitable NHS coupling agents for use in this invention have been previously described, including DSS, bis(sulfosuccinimidyl)suberate (BS³), DSP, DTSSP, SPDP, BSOCOES, DSAH, DST, and EGS, among others.

In any case, the NHS-carrier substance can now be coupled to any suitable amino-containing chloroquine substance, nucleic acid, intercalator, targeting molecule, transduction vector, or other amino-containing moiety using methods for coupling active esters described herein.

B. Thiolated Carrier Substances. Alternatively, thiolated carrier substances can be prepared from amino-containing carrier substances as described herein. Then, through disulfide coupling, the carrier substance is coupled to other available sulfhydryls on the desired thiolated intercalator, targeting molecule, transduction vector, or other moiety.

Alternatively, a sulfhydryl-containing carrier substance (i.e. thiolated PEG) is coupled to any maleimide derivative of an intercalator, transduction vector, targeting molecule, or biotin, (e.g. biotin-maleimide) or iodoacetyl derivatives such as N-iodoacetyl-N′-biotinylhexylenediamine.

C. Maleimido or Iodo-Carrier Substances. Alternatively, maleimide or iodo derivatized carrier substances, can be prepared from amino-containing carrier substances of this invention using well known methods. Such carrier substances are suitable for coupling to native or introduced sulfhydryls on the desired chloroquine substance, nucleic acid, intercalator, targeting molecule, transduction vector, or other moiety.

A maleimido group is added to an amino-carrier substance suitably prepared as described previously, by coupling a suitable hetero-bifunctional coupling agent to the available amino group. The hetero-bifunctional coupling agent consists of a suitable spacer with a maleimide group at one end and an NHS ester at the other end. Examples are previously described and include MBS, SMCC, SMPB, among others. The reaction is carried out so that the NHS ester couples to the available amino group on the carrier substance, leaving the maleimide group free for subsequent coupling to an available sulfhydryl on an intercalator, transduction vector, targeting molecule, or other moiety.

Under appropriate conditions, iodo-carrier substances (i.e. Iodo-PEG) can also be prepared for coupling to sulfhydryl groups. For instance, NHS esters of iodoacids can be coupled to the amino-carrier substances as described previously.

### PREPARATION XX

### Biotinylated Nucleic Acid Carriers

Carrier substances defined herein can be coupled to biotin by a variety of known biotinylation methods suitably modified for use with the carrier substances of this invention. For instance, an amino-containing carrier substance is combined with an active ester derivative of biotin in appropriate buffer such as 0.1 M phosphate, pH 8.0, reacting for up to 1 hour at room temperature. Examples of biotin derivatives that can be used are, biotin-N-hydroxysuccininide, biotinamidocaproate N-hydroxysuccinimide ester or sulfosuccinimidyl 2-(biotinamino)ethyl-1,3′-dithiopropionate, among others.

Through the use of suitable protection and deprotection schemes, as needed, any carrier substance of the instant invention can be coupled to biotin or a suitable derivative thereof, through any suitable coupling group. For instance, biocytin can be coupled through an available amino group to any active ester derivatized carrier substance described herein.

The resulting biotinylated carrier substance is then coupled to any suitable avidin or streptavidin that contains the desired chloroquine substance, nucleic acid, or intercalator. The avidin or streptavidin may also contain a targeting molecule, transduction vector, quinacrine or other moiety.

### PREPARATION XXI

### Avidin Nucleic Acid Carriers

Avidin or streptavidin carrier substances defined herein can be coupled to biotinylated moieties including biotinylated chloroquine substances, nucleic acids and other moieties. Biotinylated moieties can also include targeting molecules or transduction vectors. For instance, streptavidin can be suitably carboxylated without impairing the biotin binding sites. The carboxyl groups are then derivatized to provide one or more active esters as described herein. Primaquine or hydroxychloroquine amine is then coupled to the activated esters as described herein. Biotinylated moieties can be coupled to the streptavidin carrier substance before or after other moieties are coupled to the active esters.

Alternatively, moieties such as targeting molecules, intercalators or transduction vectors can be coupled to the active esters through their amino groups.

### PREPARATION XXII

### Chloroquine Substance Phosphoramidite

The prior art has shown that chloroquine given as free drug in high enough concentration, enhances the release of various agents from cellular endosomes into the cytoplasm. The purpose of these compositions is to provide the chloroquine or other chloroquine substance at the same site where the nucleic acid needs to be released, thereby reducing the overall dosage needed.

A nucleic acid carrier composition has been discovered that includes the coupling of a chloroquine substance as defined herein, to any suitable active agent carrier composition including the nucleic acid carrier compositions of this invention.

The following methods can be suitably modified for coupling certain chloroquine substances based on the disclosures of T. Koch, et al, in Bioconjugate Chem. 2000, 11, 474-483 and S. M. Gasper, et al, in JACS 119, 12762 (1997) and references therein, for the preparation of chloroquine substance phosphoramidites.

### Preparation of Primaquine Phosphoramidite

1. Primaquine carboxylic acid. In the preparation of primaquine phosphoramidite (PQ-amidite), primaquine is first derivatized to produce a carboxylic acid functional group by reacting primaquine with acetic anhydride (molar ratio 1:3) in anhydrous DMF for several hours at room temperature in the dark. The product is collected by precipitation.

Primaquine carboxamide is then prepared by reaction of primaquine carboxylic acid activated with BOP with 3-amino-1-propanol. Phosphitylation of the alcohol with ²-cyanoethyl N,N-diisopropyl phosphoramido chloridite in the presence of N,N-diisopropylethylamine produces the primaquine phosphoramidite, which is collected by precipitation from petroleum ether.

2. N-(3-hydroxypropyl)-primaquine carboxamide. To a solution of primaquine carboxylic acid (about 40 mmoles) in DMF is added an equal molar amount of BOP (benzotriazol-1-yloxy-tris(dimethylamino)-phosphonium hexafluorophosphate), and about 80 mmoles of triethylamine. The resulting mixture is stirred at room temperature for about 20 min before drop wise addition of 3-amino-1-propanol (about 40 mmoles). The reaction mixture is stirred at room temperature in the dark for 18-24 hours. The primaquine carboxamide product is collected by precipitation or column chromatography. Alternatively, if a longer extension is desired, then a longer amino-alcohol, alcohol, such as 5-amino-1-pentanol, can be substituted for the 3-amino-1-propanol.

3. PQ-Amidite. To a solution of primaquine carboxamide (about 3 mmol) in anhydrous CH₂Cl₂ is added about 7 mmoles of N,N-Diisopropylethylamine and mixed under N₂. Then about 3 mmoles of 2-cyanoethyl N,N-diisopropyl-phosphoramidochloridite is added drop wise while stirring. The reaction mixture is stirred at room temperature for 25 min. The mixture is filtrated and diluted with about 100 mL of 10% triethylamine in suitable solvent and washed with saturated aqueous NaHCO₃ (2×20 mL).

The organic solution is dried (Na₂SO₄) and evaporated under reduced pressure. The residue is dissolved in a minimum amount of CH₂Cl₂ and added drop wise to vigorously stirred ice cooled light petroleum ether (200 mL). The precipitate is collected by filtration and dried overnight under high vacuum. Alternatively, the PQ-amidite can be purified by column chromatography.

### Preparation of Hydroxychloroquine Phosphoramidite

Hydroxychloroquine phosphoramidite (HQ-amidite), can be prepared from commercially available hydroxychloroquine (7-chloro-4-(4-ethyl-(2-hydroxyethyl)-amino-1-methylbutylamino)quinoline) by reacting it with 2-cyanoethyl N,N,N′,N′-tetraisopropyl phosphorodiamidite and tetrazole.

To a stirred suspension of about 4 mmoles of hydroxychloroquine in anhydrous CH₂Cl₂ is added about 4 mmoles of 2-cyanoethyl N,N,N′,N′-tetraisopropyl-phosphorodiamidite, followed by drop wise addition of about 9 mL of 0.45 M tetrazole in CH₃CN. The reaction mixture is stirred at room temperature for 1.5-2 hours and any formed salts are removed by filtration. The filtrate is diluted with CH₂Cl₂ (50 mL) and washed with saturated aqueous NaHCO₃ (2×20 mL) and brine (20 mL). The HQ-amidite in organic solution is dried (Na₂SO₄) and collected by evaporated under reduced pressure. Alternatively, the HQ-amidite can be purified by column chromatography.

Also, if an extended linkage is desired, the hydroxychloroquine can be first derivatized to a carboxylic acid by reaction with a suitable acid anhydride. Then it is converted to a carboxamide by coupling it to the desired amino-alcohol (i.e. 5-amino-1-pentanol), as described in the preparation of primaquine carboxamide (step 2), with suitable modifications in procedure.

### Biocleavable Chloroquine Phosphoramidites

A new composition has been discovered wherein a chloroquine substance incorporated into a nucleic acid through a phosphoramidite can be subsequently released from the nucleic acid. In this composition, the linkage between the phosphoramidite and the chloroquine substance contains any suitable biocleavable linkage as defined herein. For instance, during the preparation of primaquine carboxamide (step 2), or hydroxychloroquine carboxamide, the amino-alcohol is suitably replaced with a suitable amino-alcohol that also contains a biocleavable linkage. For example, the amino alcohol can contain a disulfide bond, a hydrazone linkage or even a GFLG amino acid sequence.

In another preferred embodiment, any suitable chloroquine substance can be thiolated (Thio-Chloroquine Substance), to provide a sulfhydryl functional group. Then, one or more said Thio-Chloroquine Substances can be incorporated into any suitable nucleic acid, including antisense nucleic acids, that contain at least one thiolated phosphoramidite such as 1-O-dimethoxytrityl-hexyl-disulfide-1′-[(2-cyanoethyl)-(N,N-diisopropyl)]-phosphoramidite (Glen Research), with an available sulfhydryl group. The Thio-Chloroquine Substance is suitably coupled to the sulfhydryl on the phosphoramidite before, or after incorporation into a nucleic acid, using a disulfide exchange reaction to produce a disulfide linkage.

### Oligomerization of Chloroquine Phosphoramidites

Oligonucleotides containing one or more chloroquine phosphoramidites are synthesized on a DNA-synthesizer (Pharmacia Gene Assembler Special) using standard phosphoramidite chemistry. For example, while still on the solid support, the 5′-OH terminus of an oligonucleotide is suitably coupled with an HEG (hexaethyloxy glycol, KJ-Ross-Petersen A/S, Denmark), linker followed by coupling with either PQ-amidite or HQ-amidite. The chloroquine phosphoramidite oligonucleotide conjugates are cleaved from the solid support and the nucleobase protection groups removed by treatment with aqueous ammonium hydroxide. The crude chloroquine substance-modified oligonucleotides are suitably purified by reversed-phase HPLC.

Preferred oligonucleotides and oligodeoxynucleotides containing chloroquine substance-phosphoramidites include any suitable nucleic acid described herein, especially therapeutic nucleic acids such as antisense oligodeoxynucleotides, siRNAs and combinations of nucleic acids coupled to peptides, transduction vectors and/or carrier substances as described herein.

### PREPARATION XXIII

### Amino Acid-Coupled Chloroquine Substance

The purpose of these compositions is to deliver the chloroquine or other chloroquine substance at the same site as its peptide or protein carrier, thereby reducing the overall dosage needed. A peptide carrier composition has been discovered that includes the coupling of a chloroquine substance as defined herein, to any suitable transduction vector or peptide carrier composition of this invention. The following methods can be suitably modified for coupling amino derivatized chloroquine substances based on the disclosures of Z. Wang, et al, in JACS 117, 5438-5444 (1995) and references therein, for the preparation of amino acid-coupled chloroquine substances.

### Primaquine-Coupled N-alpha-Fmoc-L-Aspartic Acid alpha-Tert-Butyl Ester

1. Activated Ester N-alpha-Fmoc-aspartic acid alpha-tert-butyl ester. To prepare the activated aspartic acid ester, 1-hydroxybenzotriazole (HOBt) (0.5 mmoles), dissolved in about 2 mL of dry DMF is added to an ice-cooled solution of N-alpha-Fmoc-aspartic acid alpha-tert-butyl ester (0.5 mmoles) in about 2 mL of dry dichloromethane, followed by the addition of DCC (dicyclohexyl carbodiimide, 0.5 mmoles) in 2 mL of dry dichloromethane.

The reaction mixture is stirred at 0° C. for 1 h then at room temperature for 2 hours. The reaction mixture is filtered and activated ester is collected from the filtrate that is evaporated to dryness. The activated ester is redissolved in about 4 mL of dry dichloromethane.

2. Coupling to Primaquine. To form a free base, primaquine HCl salt (0.4 mmoles) in dry DMF is mixed with N,N-diisopropylethylamine (0.4 mmoles) and stirred at room temperature for 2-5 minutes.

The coupling reaction is started by adding the free base of primaquine (PQ) to the activated ester solution. The final pH of the coupling reaction is adjusted to 8.0 by the addition of about 0.05 mL of diisopropylethylamine, and the mixture is stirred for about 20 minutes. The reaction mixture is concentrated to dryness under reduced pressure. The primaquine-coupled aspartic acid tert butyl ester is purified by recrystallization in suitable solvent (i.e. methanol) and dried. Alternatively, the product can be purified by column chromatography.

3. Primaquine-Coupled Fmoc-L-Aspartic Acid. To prepared primaquine-coupled Fmoc-L-aspartic acid (PQ-Fmoc-aspartate), the PQ-coupled aspartic acid tert-butyl ester (0.3 mmol.) is dissolved in dry dichloromethane or other suitable solvent and cooled to 0° C. To this solution is added about 2 mL of trifluoroacetic acid and stirring is continued at 0° C. for about 2 hours, followed by stirring at room temperature until the tert-butyl ester is removed. The reaction mixture is concentrated under reduced pressure without heating to dryness. The PQ-Fmoc-aspartate is purified by recrystallization in suitable solvent (i.e. methanol) and dried. Alternatively, the product can be purified by column chromatography.

4. Primaquine-Coupled Transduction Vector Peptide. All Fmoc-amino acids, piperidine, 4-(dimethyl-amino)pyridine, dichloromethane, DMF, HOBT, 2-(1H-benzotriazol-1-yl)-1,1,3,3-tetramethyl-uronium hexafluorophosphate (HBTU), N,N-diisopropyl ethylamine and HMP-linked polystyrene resin are available from Applied Biosystems Division, Perkin-Elmer. Trifluoroacetic acid, 1,2-ethanedithiol, phenol and thioanisol are available from Sigma.

One or more PQ-Fmoc-aspartate moieties can be incorporated into any suitable peptide including transduction vector peptides (i.e. Tat-derived from amino acids 42-72). For instance, the desired transduction vector peptide is first synthesized on an Applied Biosystems 431A peptide synthesizer using standard FastMoc protocols. Then primaquine attachment to the N-terminus of the transduction vector peptide is achieved by using PQ-Fmoc-aspartate (step 3, above) and standard FastMoc coupling reagents. Cleavage and deprotection of the peptide are carried out in 2 mL of Reagent K for 6 h at room temperature. Reagent K contains 1.75 mL of TFA, 0.10 mL of thioanisole, 0.10 mL of water and 0.05 mL of 1,2-ethanedithiol. After cleavage from the resin, the PQ-TV-peptide is purified by HPLC.

With suitable modifications in these methods, other amino-containing chloroquine substances can be substituted for the primaquine HCl. Some substitution examples are primaquine diphosphate, amino-hydroxychloroquine and amino-derivatized amodiaquine. Also, with suitable modifications in these methods, other suitable Fmoc-amino acids can be substituted for the Fmoc-aspartate.

In another preferred embodiment, any suitable chloroquine substance can be thiolated (Thio-Chloroquine Substance), to provide a sulfhydryl functional group. Then, one or more said Thio-Chloroquine Substances can be incorporated into any suitable peptide, including transduction vector peptides, or carrier substances that contain at least one cysteine amino acid. The Thio-Chloroquine Substance is suitably coupled to the cysteine using a disulfide exchange reaction to produce a disulfide linkage.

While the invention has been described with reference to certain specific embodiments, it is understood that changes may be made by one skilled in the art that would not thereby depart from the spirit and scope of the invention, which is limited only by the claims appended hereto.

What is claimed is:

1. A chloroquine-coupled nucleic acid composition comprising;
a) a chloroquine substance covalently coupled to;
b) a nucleic acid.

2. The composition of claim 1 wherein said chloroquine substance (a) is selected from the group consisting of quinoline compounds, 4-aminoquinoline compounds, 2-phenylquinoline compounds, chloroquines, hydroxychloroquines, amodiaquins, amopyroquines, halofantrines, mefloquines, nivaquines, primaquines, quinone imines, chloroquine analogs or derivatives, (−)-enantiomers of chloroquine, (−)-enantiomers of hydroxychloroquine and amino, thio, phenyl, alkyl, vinyl and halogen derivatives thereof.

3. The composition of claim 1 wherein said nucleic acid is selected from the group consisting of single stranded RNA, double stranded RNA, antisense RNA, messenger RNA, transfer RNA, small interfering RNA, micro RNA, hairpin RNA, ribozymes, riboswitches, 5′ derivatized RNA, 3′ derivatized RNA, backbone derivatized RNA, single stranded DNA, double stranded DNA, 5′ derivatized DNA, 3′ derivatized DNA, DNAzymes, oligonucleotides, phosphodiester sense and antisense oligonucleotides, phosphodiester sense and antisense oligodeoxynucleotides, backbone derivatized sense and antisense oligonucleotides, backbone derivatized sense and antisense oligodeoxynucleotides, mixed backbone derivatized sense and antisense oligonucleotides, mixed backbone derivatized sense and antisense oligodeoxynucleotides, RNA-DNA hybrids, modified ribose nucleic acids, locked nucleic acids, triplex-forming oligonucleotides, RNA-DNA chimeras, sense and antisense peptide nucleic acids, PNA clamps and phosphoric acid ester nucleic acids.

4. The composition of claim 1 further comprising a targeting molecule coupled to said carrier substance.

5. The composition of claim 1 further comprising a transduction vector coupled to said carrier substance.

6. The composition of claim 1 wherein said covalent coupling of said chloroquine substance of (a) to nucleic acid (b) is a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

7. The composition of claim 1 further comprising an intercalator coupled to said carrier substance.

8. A chloroquine-coupled nucleic acid composition comprising;
a) a chloroquine substance covalently coupled to;
b) a carrier substance and;
c) wherein said carrier substance is coupled to a nucleic acid.

9. The composition of claim 8 wherein said chloroquine substance (a) is selected from the group consisting of quinoline compounds, 4-aminoquinoline compounds, 2-phenylquinoline compounds, chloroquines, hydroxychloroquines, amodiaquins, amopyroquines, halofantrines, mefloquines, nivaquines, primaquines, quinone imines, chloroquine analogs or derivatives, (−)-enantiomers of chloroquine, (−)-enantiomers of hydroxychloroquine and amino, thio, phenyl, alkyl, vinyl and halogen derivatives thereof.

10. The composition of claim 8 wherein said nucleic acid is selected from the group consisting of single stranded RNA, double stranded RNA, antisense RNA, messenger RNA, transfer RNA, small interfering RNA, micro RNA, hairpin RNA, ribozymes, riboswitches, 5′ derivatized RNA, 3′ derivatized RNA, backbone derivatized RNA, single stranded DNA, double stranded DNA, 5′ derivatized DNA, 3′ derivatized DNA, DNAzymes, oligonucleotides, phosphodiester sense and antisense oligonucleotides, phosphodiester sense and antisense oligodeoxynucleotides, backbone derivatized sense and antisense oligonucleotides, backbone derivatized sense and antisense oligodeoxynucleotides, mixed backbone derivatized sense and antisense oligonucleotides, mixed backbone derivatized sense and antisense oligodeoxynucleotides, RNA-DNA hybrids, modified ribose nucleic acids, locked nucleic acids, triplex-forming oligonucleotides, RNA-DNA chimeras, sense and antisense peptide nucleic acids, PNA clamps and phosphoric acid ester nucleic acids.

11. The composition of claim 8 wherein said carrier substance is selected from the group consisting of avidins, streptavidins, liposomes, micelles and dendrimers.

12. The composition of claim 8 further comprising a targeting molecule coupled to said carrier substance.

13. The composition of claim 8 further comprising a transduction vector coupled to said carrier substance.

14. The composition of claim 8 wherein said covalent coupling of said chloroquine substance of (a) to carrier substance (b) is a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

15. A method for synthesizing a chloroquine substance-coupled nucleic acid composition comprising the steps of coupling;
a) a chloroquine substance to;
b) a nucleic acid.

16. The method of claim 15 wherein said coupling of chloroquine substance of (a) to said nucleic acid of (b) includes a biocleavable linkage selected from the group consisting of an acid labile linkage, a disulfide linkage, a protected disulfide linkage, an ester linkage, an ortho ester linkage, a phosphonamide linkage, a biocleavable peptide linkage, an azo linkage and an aldehyde bond.

17. The method of claim 15 wherein said chloroquine substance of (a) is selected from the group consisting of quinoline compounds, 4-aminoquinoline compounds, 2-phenylquinoline compounds, chloroquines, hydroxychloroquines, amodiaquins, amopyroquines, halofantrines, mefloquines, nivaquines, primaquines, quinone imines, chloroquine analogs or derivatives, (−)-enantiomers of chloroquine, (−)-enantiomers of hydroxychloroquine and amino, thio, phenyl, alkyl, vinyl and halogen derivatives thereof.

18. The method of claim 15 further comprising the step of coupling a targeting molecule to said nucleic acid.

19. The method of claim 15 further comprising the step of coupling a transduction vector to said nucleic acid.

20. The method of claim 15 further comprising the step of coupling a quinacrine to said nucleic acid.
